(19) United States
(12) Patent Application Publication
(10) Pub. No.: US 2023/0106830 A1
(43) Pub. Date: Apr. 6, 2023

(54) TRANSFER FILM, METHOD FOR PRODUCING LAMINATE, AND BLOCKED ISOCYANATE COMPOUND

(71) Applicant: FUJIFILM Corporation, Tokyo (JP)

(72) Inventors: Yohei ARITOSHI, Fujinomiya-shi (JP); Kentaro TOYOOKA, Fujinomiya-shi (JP); Kunihiko KODAMA, Fujinomiya-shi (JP)

(73) Assignee: FUJIFILM Corporation, Tokyo (JP)

(21) Appl. No.: 18/058,814

(22) Filed: Nov. 25, 2022

Related U.S. Application Data
(63) Continuation of application No. PCT/JP2021/019753, filed on May 25, 2021.

(30) Foreign Application Priority Data
May 27, 2020 (JP) 2020-091974
Mar. 23, 2021 (JP) 2021-048128

(51) Int. Cl. G03F 7/033; C07C 265/14

(52) U.S. Cl. CPC G03F 7/033; C07C 265/14

(57) ABSTRACT

An object of the present invention is to provide a transfer film capable of suppressing a corrosion of a wiring line and an electrode. In addition, an object of the present invention is to provide a method for producing a laminate using the transfer film. In addition, an object of the present invention is to provide a novel blocked isocyanate compound. The transfer film of the present invention has a temporary support and a photosensitive composition layer disposed on the temporary support, in which the photosensitive composition layer includes an alkali-soluble resin, a polymerizable compound, a polymerization initiator, and a blocked isocyanate compound having an NCO value of 4.5 mmol/g or more.

### CROSS-REFERENCE TO RELATED APPLICATIONS

This application is a Continuation of PCT International Application No. PCT/JP2021/019753 filed on May 25, 2021, which claims priority under 35 U.S.C. § 119(a) to Japanese Patent Application No. 2020-091974 filed on May 27, 2020 and Japanese Patent Application No. 2021-048128 filed on Mar. 23, 2021. The above applications are hereby expressly incorporated by reference, in its entirety, into the present application.

### BACKGROUND OF THE INVENTION

### 1. Field of the Invention

The present invention relates to a transfer film, a method for producing a laminate, and a blocked isocyanate compound.

### 2. Description of the Related Art

From the viewpoint that the number of steps for obtaining a predetermined pattern is small, a method in which, using a transfer film, a photosensitive composition layer provided on any substrate is exposed through a mask and then developed has been widely used.

The transfer film having the photosensitive composition layer may be used for forming a protective film (touch panel electrode protective film) for protecting a sensor electrode and a lead wire in a touch panel. For example, JP2020-071372A discloses a photosensitive resin film (photosensitive composition layer) including an alkali-soluble resin, a polymerizable compound having an unsaturated double bond, a photopolymerization initiator, a coloring material, and a blocked isocyanate compound as a thermal crosslinking agent.

### SUMMARY OF THE INVENTION

In recent years, there is a demand for further improvement in the performance of the touch panel electrode protective film, and specifically, there is a demand for a touch panel electrode protective film in which a corrosion of the sensor electrode and the lead wire in the touch panel can be suppressed.

In a case where the present inventors use the transfer film having the photosensitive composition layer as disclosed in JP2020-071372A to form a touch panel electrode protective film, it has been found that the corrosion of the wiring line and the electrode cannot be suppressed in some cases depending on the type of the blocked isocyanate compound included in the photosensitive composition layer, and that there is room for improvement.

Therefore, an object of the present invention is to provide a transfer film capable of suppressing a corrosion of a wiring line and an electrode. Another object of the present invention is to provide a method for producing a laminate using the transfer film. Another object of the present invention is to provide a novel blocked isocyanate compound.

The present inventors have conducted intensive studies on the above-described objects, and as a result, have found that the above-described objects can be accomplished by the following configurations.

[1]

A transfer film comprising:

a temporary support; and

a photosensitive composition layer disposed on the temporary support,

in which the photosensitive composition layer includes an alkali-soluble resin, a polymerizable compound, a polymerization initiator, and a blocked isocyanate compound having an NCO value of 4.5 mmol/g or more.

[2]

The transfer film according to [1],

in which the NCO value of the blocked isocyanate compound is more than 5.0 mmol/g.

[3]

The transfer film according to [1] or [2],

in which the blocked isocyanate compound has a ring structure.

[4]

The transfer film according to any one of [1] to [3],

in which the blocked isocyanate compound is a blocked isocyanate compound represented by Formula Q,

B¹-A¹-L¹-A²-B² Formula Q

in Formula Q, B¹ and B² each independently represent a blocked isocyanate group, A¹ and A² each independently represent a single bond or an alkylene group having 1 to 10 carbon atoms, and L¹ represents a divalent linking group.

[5]

The transfer film according to any one of [1] to [4],

in which the blocked isocyanate compound is a blocked isocyanate compound represented by Formula QA,

B^{1a}-A^{1a}-L^{1a}-A^{2a}-B^{2a} Formula QA

in Formula QA, B^{1a} and B^{2a} each independently represent a blocked isocyanate group, A^{1a} and A^{2a} each independently represent a divalent linking group, and L^{1a} represents a cyclic divalent saturated hydrocarbon group or a divalent aromatic hydrocarbon group.

[6]

The transfer film according to any one of [1] to [5],

in which the photosensitive composition layer further includes a blocked isocyanate compound having an NCO value of less than 4.5 mmol/g.

[7]

The transfer film according to any one of [1] to [6],

in which the alkali-soluble resin includes a structural unit derived from a vinylbenzene derivative, a structural unit having a radically polymerizable group, and a structural unit having an acid group, and

a content of the structural unit derived from the vinylbenzene derivative is 35% by mass or more with respect to a total amount of all structural units included in the alkali-soluble resin.

[8]

The transfer film according to [7],

in which the content of the structural unit derived from the vinylbenzene derivative is 45% by mass or more with respect to the total amount of all structural units included in the alkali-soluble resin.

[9]

The transfer film according to any one of [1] to [8], further comprising:

a refractive index-adjusting layer,

in which the refractive index-adjusting layer is disposed in contact with the photosensitive composition layer, and

a refractive index of the refractive index-adjusting layer is 1.60 or more.

[10]

The transfer film according to any one of [1] to [9],

in which the photosensitive composition layer is used for forming a touch panel electrode protective film.

[11]

A method for producing a laminate, comprising:

an affixing step of bringing the photosensitive composition layer on the temporary support of the transfer film according to any one of [1] to [10] into contact with a substrate having a conductive layer to affix the photosensitive composition layer to the substrate and obtain a photosensitive composition layer-attached substrate having the substrate, the conductive layer, the photosensitive composition layer, and the temporary support in this order;

an exposing step of exposing the photosensitive composition layer in a patterned manner; and

a developing step of developing the exposed photosensitive composition layer to form a pattern,

in which the producing method further includes, between the affixing step and the exposing step or between the exposing step and the developing step, a peeling step of peeling the temporary support from the substrate with a photosensitive composition layer.

[12]

A transfer film comprising:

a temporary support; and

a photosensitive composition layer disposed on the temporary support,

in which the photosensitive composition layer includes an alkali-soluble resin, a polymerizable compound, a polymerization initiator, and a blocked isocyanate compound, and

an NCO value of the photosensitive composition layer is more than 0.50 mmol/g.

[13]

A blocked isocyanate compound represented by Formula QA,

B^{1a}-A^{1a}-L^{1a}-A^{2a}-B^{2a} Formula QA

in Formula QA, B^{1a} and B^{2a} each independently represent a blocked isocyanate group, A^{1a} and A^{2a} each independently represent a divalent linking group, and L^{1a} represents a cyclic divalent saturated hydrocarbon group or a divalent aromatic hydrocarbon group.

[14]

The blocked isocyanate compound according to [13],

in which the blocked isocyanate compound is represented by Formula Q-1 described later.

[15]

The blocked isocyanate compound according to [14],

in which a mass ratio of a cis form to a trans form is cis form/trans form=10/90 to 90/10.

According to the present invention, it is possible to provide a transfer film capable of suppressing a corrosion of a wiring line and an electrode. In addition, according to the present invention, it is possible to provide a method for producing a laminate using the transfer film. In addition, according to the present invention, it is possible to provide a novel blocked isocyanate compound.

### BRIEF DESCRIPTION OF THE DRAWINGS

FIG. 1 is a schematic cross-sectional view showing a specific example of a touch panel to which the transfer film according to the embodiment of the present invention can be applied.

FIG. 2 is a schematic cross-sectional view showing a specific example of a touch panel to which the transfer film according to the embodiment of the present invention can be applied.

FIG. 3 is a schematic plan view showing a specific example of a touch panel to which the transfer film according to the embodiment of the present invention can be applied.

FIG. 4 is a cross-sectional view taken along a line A-A of FIG. 3.

### DESCRIPTION OF THE PREFERRED EMBODIMENTS

Hereinafter, the details of the present invention will be described.

In the present specification, a numerical value range indicated by using “to” means a range including the numerical values before and after “to” as the lower limit value and the upper limit value, respectively.

In addition, regarding numerical ranges that are described stepwise in the present specification, an upper limit value or a lower limit value described in a numerical range may be replaced with an upper limit value or a lower limit value of another stepwise numerical range. In addition, in the range of numerical values described in the present specification, an upper limit value and a lower limit value disclosed in a certain range of numerical values may be replaced with values shown in Examples.

Further, a term “step” in the present specification indicates not only an independent step but also a step which cannot be clearly distinguished from other steps as long as the intended purpose of the step is achieved.

In the present specification, a term “transparent” means that an average transmittance of visible light at a wavelength of 400 to 700 nm is 80% or more, and preferably 90% or more. In addition, the average transmittance of visible light is a value measured using a spectrophotometer, and can be measured, for example, using a spectrophotometer U-3310 manufactured by Hitachi, Ltd.

A weight-average molecular weight (Mw) and a number-average molecular weight (Mn) in the present disclosure are molecular weights in terms of polystyrene used as a standard substance, which are detected by using tetrahydrofuran (THF), a differential refractometer, and a gel permeation chromatography (GPC) analyzer using TSKgel GMHxL, TSKgel G4000HxL, and TSKgel G2000HxL (all product names manufactured by Tosoh Corporation) as columns, unless otherwise specified.

In the present disclosure, unless otherwise specified, a molecular weight of a compound having a molecular weight distribution is the weight-average molecular weight.

In addition, in the present specification, a refractive index is a value measured with an ellipsometer at a wavelength of 550 nm unless otherwise specified.

In the present specification, “(meth)acrylic” has a concept including both acrylic and methacrylic, “(meth)acrylate” has a concept including both acrylate and methacrylate, and “(meth)acryloxy group” has a concept including both acryloxy group and methacryloxy group.

### First Embodiment of Transfer Film

The transfer film according to a first embodiment of the present invention (hereinafter, also referred to as a “first transfer film”) has a temporary support and a photosensitive composition layer disposed on the temporary support, in which the photosensitive composition layer includes an alkali-soluble resin, a polymerizable compound, a polymerization initiator, and a blocked isocyanate compound having an NCO value of 4.5 mmol/g or more. Hereinafter, the blocked isocyanate compound having an NCO value of 4.5 mmol/g or more is also referred to as a “first blocked isocyanate compound”.

A feature point of the first transfer film is that the photosensitive composition layer having the first transfer film includes the first blocked isocyanate compound.

Here, examples of a method for forming a protective film using the first transfer film include a method in which a substrate having a conductive layer (sensor electrode, lead wire, and the like) or the like is brought into contact with the first transfer film to affix the substrate to the first transfer film, and through steps such as pattern exposure of the photosensitive composition layer having the first transfer film, development, and post-baking, a protective film in a patterned shape is formed.

The alkali-soluble resin included in the photosensitive composition layer is required from the viewpoint of developability of the photosensitive composition layer, but the present inventors have found that corrosion of the conductive layer may be caused by an action of an acid group included in the alkali-soluble resin, such as a carboxy group.

In response to this problem, the present inventors have found that the corrosion of the conductive layer can be suppressed by using the first blocked isocyanate compound.

It is presumed that the reason for this is that the post-baking step generates a sufficient amount of isocyanate groups from the blocked isocyanate compound to react with the acid group of the alkali-soluble resin, and as a result, the corrosion of the conductive layer can be suppressed.

Hereinafter, each member constituting the first transfer film will be described.

<Temporary Support>

The first transfer film has a temporary support. The temporary support is a member which supports the photosensitive composition layer described later, and the like, and is finally removed by a peeling treatment.

The temporary support is preferably a film and more preferably a resin film. As the temporary support, a film which has flexibility and does not show significant deformation, contraction, or stretching under pressure or under pressure and heating can be used.

Examples of such a film include a polyethylene terephthalate film (for example, a biaxially stretching polyethylene terephthalate film), a cellulose triacetate film, a polystyrene film, a polyimide film, and a polycarbonate film.

Among these, as the temporary support, a biaxially stretching polyethylene terephthalate film is preferable.

In addition, it is preferable that the film used as the temporary support does not have deformations such as a wrinkles, a scratch, and the like.

From the viewpoint that pattern exposure through the temporary support can be performed, it is preferable that the temporary support has high transparency, and the transmittance at 365 nm is preferably 60% or more and more preferably 70% or more.

From the viewpoint of pattern forming properties during the pattern exposure through the temporary support and transparency of the temporary support, it is preferable that a haze of the temporary support is small. Specifically, a haze value of the temporary support is preferably 2% or less, more preferably 0.5% or less, and still more preferably 0.1% or less.

From the viewpoint of the pattern forming properties during the pattern exposure through the temporary support and the transparency of the temporary support, it is preferable that the number of fine particles, foreign substances, and defects included in the temporary support is small. The number of fine particles, foreign substances, and defects having a diameter of 1 μm or more is preferably 50 pieces/10 mm² or less, more preferably 10 pieces/10 mm² or less, still more preferably 3 pieces/10 mm² or less, and particularly preferably 0 pieces/10 mm².

A thickness of the temporary support is not particularly limited, but from the viewpoint of easiness of handling and general-purpose properties, is preferably 5 to 200 μm, more preferably 10 to 150 μm, and still more preferably 10 to 50 μm.

From the viewpoint of imparting handleability, a layer (lubricant layer) containing fine particles may be provided on a surface of the temporary support. The lubricant layer may be provided on one surface of the temporary support or on both surfaces thereof. A diameter of the particles contained in the lubricant layer may be 0.05 to 0.8 μm. In addition, a film thickness of the lubricant layer may be 0.05 to 1.0 μm.

Examples of the temporary support include a biaxially stretching polyethylene terephthalate film having a film thickness of 16 μm, a biaxially stretching polyethylene terephthalate film having a film thickness of 12 μm, and a biaxially stretching polyethylene terephthalate film having a film thickness of 9 μm.

For example, preferred aspects of the temporary support are described in paragraphs [0017] and [0018] of JP2014-085643A, paragraphs [0019] to [0026] of JP2016-027363A, paragraphs [0041] to [0057] of WO2012/081680A, and paragraphs [0029] to [0040] of WO2018/179370A, the contents of which are incorporated herein by reference.

<Photosensitive Composition Layer>

The first transfer film has a photosensitive composition layer. A pattern can be formed on an object to be transferred by transferring the photosensitive composition layer onto the object to be transferred and then exposing and developing the photosensitive composition layer.

The photosensitive composition layer includes an alkali-soluble resin, a polymerizable compound, a polymerization initiator, and the first blocked isocyanate compound.

The photosensitive composition layer may be a positive tone or a negative tone.

The positive tone photosensitive composition layer is a photosensitive composition layer having a solubility in a developer that increases by exposure to an exposed portion, and the negative tone photosensitive composition layer is a photosensitive composition layer having a solubility in a developer that decreases by exposure to an exposed portion.

Among these, it is preferable to use a negative tone photosensitive composition layer. In a case where the photosensitive composition layer is a negative tone photosensitive composition layer, a pattern to be formed corresponds to a cured film.

Hereinafter, the components included in the negative tone photosensitive composition layer will be described in detail.

[Polymerizable Compound]

The photosensitive composition layer includes a polymerizable compound.

The polymerizable compound is a compound having a polymerizable group. Examples of the polymerizable group include a radically polymerizable group and a cationically polymerizable group, and a radically polymerizable group is preferable.

The polymerizable compound preferably includes a radically polymerizable compound having an ethylenically unsaturated group (hereinafter, also simply referred to as an “ethylenically unsaturated compound”).

As the ethylenically unsaturated group, a (meth)acryloxy group is preferable.

The ethylenically unsaturated compound preferably includes a bi- or higher functional ethylenically unsaturated compound. Here, the “bi- or higher functional ethylenically unsaturated compound” means a compound having two or more ethylenically unsaturated groups in one molecule.

As the ethylenically unsaturated compound, a (meth)acrylate compound is preferable.

From the viewpoint of film hardness after curing, for example, the ethylenically unsaturated compound preferably includes a bifunctional ethylenically unsaturated compound (preferably a bifunctional (meth)acrylate compound) and a tri- or higher functional ethylenically unsaturated compound (preferably a tri- or higher functional (meth)acrylate compound).

Examples of the bifunctional ethylenically unsaturated compound include tricyclodecane dimethanol di(meth)acrylate, 1,9-nonanediol di(meth)acrylate, 1,10-decanediol di(meth)acrylate, and 1,6-hexanediol di(meth)acrylate.

Examples of a commercially available product of the bifunctional ethylenically unsaturated compound include tricyclodecane dimethanol diacrylate [product name: NK ESTER A-DCP, Shin-Nakamura Chemical Co., Ltd.], tricyclodecane dimethanol dimethacrylate [product name: NK ESTER DCP, Shin-Nakamura Chemical Co., Ltd.], 1,9-nonanediol diacrylate [product name: NK ESTER A-NOD-N, Shin-Nakamura Chemical Co., Ltd.], 1,10-decanediol diacrylate [product name: NK ESTER A-DOD-N, Shin-Nakamura Chemical Co., Ltd.], and 1,6-hexanediol diacrylate [product name: NK ESTER A-HD-N, Shin-Nakamura Chemical Co., Ltd.].

Examples of the tri- or higher functional ethylenically unsaturated compound include dipentaerythritol (tri/tetra/penta/hexa)(meth)acrylate, pentaerythritol (tri/tetra)(meth)acrylate, trimethylolpropane tri(meth)acrylate, ditrimethylolpropane tetra(meth)acrylate, isocyanuric acid (meth)acrylate, and glycerin tri(meth)acrylate.

Here, the “(tri/tetra/penta/hexa)(meth)acrylate” is a concept including tri(meth)acrylate, tetra(meth)acrylate, penta(meth)acrylate, and hexa(meth)acrylate. In addition, the “(tri/tetra)(meth)acrylate” is a concept including tri(meth)acrylate and tetra(meth)acrylate. The tri- or higher functional ethylenically unsaturated compound is not particularly limited in the upper limit of the number of functional groups, but the number of functional groups can be, for example, 20 or less, or can be 15 or less.

Examples of a commercially available product of the tri- or higher functional ethylenically unsaturated compound include dipentaerythritol hexaacrylate [product name: KAYARAD DPHA, Shin-Nakamura Chemical Co., Ltd.].

The ethylenically unsaturated compound more preferably includes 1,9-nonanediol di(meth)acrylate or 1,10-decanediol di(meth)acrylate, and dipentaerythritol (tri/tetra/penta/hexa)(meth)acrylate.

Examples of the ethylenically unsaturated compound also include a caprolactone-modified compound of a (meth)acrylate compound [KAYARAD (registered trademark) DPCA-20 of Nippon Kayaku Co., Ltd., A-9300-1CL of Shin-Nakamura Chemical Co., Ltd., or the like], an alkylene oxide-modified compound of a (meth)acrylate compound [KAYARAD (registered trademark) RP-1040 of Nippon Kayaku Co., Ltd., ATM-35E or A-9300 of Shin-Nakamura Chemical Co., Ltd., EBECRYL (registered trademark) 135 of Daicel-Allnex Ltd., or the like], and ethoxylated glycerin triacrylate [NK ESTER A-GLY-9E of Shin-Nakamura Chemical Co., Ltd., or the like].

Examples of the ethylenically unsaturated compound also include a urethane (meth)acrylate compound. As the urethane (meth)acrylate compound, a tri- or higher functional urethane (meth)acrylate compound is preferable. Examples of the tri- or higher functional urethane (meth)acrylate compound include 8UX-015A [Taisei Fine Chemical Co., Ltd.], NK ESTER UA-32P [Shin-Nakamura Chemical Co., Ltd.], and NK ESTER UA-1100H [Shin-Nakamura Chemical Co., Ltd.].

From a viewpoint of improving developability, the ethylenically unsaturated compound preferably includes an ethylenically unsaturated compound having an acid group.

Examples of the acid group include a phosphoric acid group, a sulfonic acid group, and a carboxy group. Among these, as the acid group, a carboxy group is preferable.

Examples of the ethylenically unsaturated compound having an acid group include a tri- or tetrafunctional ethylenically unsaturated compound having an acid group [compound obtained by introducing a carboxy group to pentaerythritol tri- and tetraacrylate (PETA) skeletons (acid value: 80 to 120 mgKOH/g)], and a penta- or hexafunctional ethylenically unsaturated compound having an acid group [compound obtained by introducing a carboxy group to a dipentaerythritol penta- or hexaacrylate (DPHA) skeleton (acid value: 25 to 70 mgKOH/g)]. The tri- or higher functional ethylenically unsaturated compound having an acid group may be used in combination with the bifunctional ethylenically unsaturated compound having an acid group, as necessary.

As the ethylenically unsaturated compound having an acid group, at least one compound selected from the group consisting of a bi- or higher functional ethylenically unsaturated compound having a carboxy group and a carboxylic acid anhydride thereof is preferable. In a case where the ethylenically unsaturated compound having an acid group is at least one compound selected from the group consisting of a bi- or higher functional ethylenically unsaturated compound having a carboxy group and a carboxylic acid anhydride thereof, the developability and the film hardness are further enhanced.

Examples of the bi- or higher functional ethylenically unsaturated compound having a carboxy group include ARONIX (registered trademark) TO-2349 [Toagosei Co., Ltd.], ARONIX (registered trademark) M-520 [Toagosei Co., Ltd.], and ARONIX (registered trademark) M-510 [Toagosei Co., Ltd.].

As the ethylenically unsaturated compound having an acid group, polymerizable compounds having an acid group, which are described in paragraphs [0025] to [0030] of JP2004-239942A, can be preferably used, and the contents described in this publication are incorporated herein by reference.

A molecular weight of the ethylenically unsaturated compound is preferably 200 to 3,000, more preferably 250 to 2,600, still more preferably 280 to 2,200, and particularly preferably 300 to 2,200.

A content of the ethylenically unsaturated compound having a molecular weight of 300 or less among the ethylenically unsaturated compounds is preferably 30% by mass or less, more preferably 25% by mass or less, and still more preferably 20% by mass or less with respect to a content of all ethylenically unsaturated compounds included in the photosensitive composition layer.

The photosensitive composition layer may include only one kind of polymerizable compound, or may include two or more kinds of polymerizable compounds.

A content of the polymerizable compound (preferably, the ethylenically unsaturated compound) is preferably 1% to 70% by mass, more preferably 10% to 70% by mass, still more preferably 20% to 60% by mass, and particularly preferably 20% to 50% by mass with respect to the total mass of the photosensitive composition layer.

In a case where the photosensitive composition layer includes the bi- or higher functional ethylenically unsaturated compound, the photosensitive composition layer may further include a monofunctional ethylenically unsaturated compound.

In a case where the photosensitive composition layer includes the bi- or higher functional ethylenically unsaturated compound, it is preferable that the bi- or higher functional ethylenically unsaturated compound is a main component of ethylenically unsaturated compounds included in the photosensitive composition layer.

In a case where the photosensitive composition layer includes the bi- or higher functional ethylenically unsaturated compound, a content of the bi- or higher functional ethylenically unsaturated compound is preferably 60% to 100% by mass, more preferably 80% to 100% by mass, and still more preferably 90% to 100% by mass with respect to the content of all ethylenically unsaturated compounds included in the photosensitive composition layer.

In a case where the photosensitive composition layer includes the ethylenically unsaturated compound having an acid group (preferably, the bi- or higher functional ethylenically unsaturated compound having a carboxy group or the carboxylic acid anhydride thereof), the content of the ethylenically unsaturated compound having an acid group is preferably 1% to 50% by mass, more preferably 1% to 20% by mass, and still more preferably 1% to 10% by mass with respect to the total mass of the photosensitive composition layer.

[Polymerization Initiator]

The photosensitive composition layer includes a polymerization initiator.

As the polymerization initiator, a photopolymerization initiator is preferable.

Examples of the photopolymerization initiator include a photopolymerization initiator having an oxime ester structure (hereinafter also referred to as an “oxime-based photopolymerization initiator”), a photopolymerization initiator having an α-aminoalkylphenone structure (hereinafter also referred to as an “α-aminoalkylphenone-based photopolymerization initiator”), a photopolymerization initiator having an α-hydroxyalkylphenone structure (hereinafter also referred to as an “α-hydroxyalkylphenone-based polymerization initiator”), a photopolymerization initiator having an acylphosphine oxide structure (hereinafter also referred to as an “acylphosphine oxide-based photopolymerization initiator”), and a photopolymerization initiator having an N-phenylglycine structure (hereinafter also referred to as an “N-phenylglycine-based photopolymerization initiator”).

The photopolymerization initiator preferably includes at least one kind selected from the group consisting of the oxime-based photopolymerization initiator, the α-aminoalkylphenone-based photopolymerization initiator, the α-hydroxyalkylphenone-based polymerization initiator, and the N-phenylglycine-based photopolymerization initiator, and more preferably includes at least one kind selected from the group consisting of the oxime-based photopolymerization initiator, the α-aminoalkylphenone-based photopolymerization initiator, and the N-phenylglycine-based photopolymerization initiator.

In addition, as the photopolymerization initiator, for example, polymerization initiators disclosed in paragraphs [0031] to [0042] of JP2011-095716A and paragraphs [0064] to [0081] of JP2015-014783A may be used.

Examples of a commercially available product of the photopolymerization initiator include 1-[4-(phenylthio)]phenyl-1,2-octanedione-2-(O-benzoyloxime) [product name: IRGACURE (registered trademark) OXE-01, manufactured by BASF SE], 1-[9-ethyl-6-(2-methylbenzoyl)-9H-carbazol-3-yl]ethanone-1-(O-acetyloxime) [product name: IRGACURE (registered trademark) OXE-02, manufactured by BASF SE], 8-[5-(2,4,6-trimethylphenyl)-11-(2-ethylhexyl)-11H-benzo[a]carbazoyl][2-(2,2,3,3-tetrafluoro propoxy)phenyl]methanone-(O-acetyloxime) [product name: IRGACURE (registered trademark) OXE-03, manufactured by BASF SE], 1-[4-[4-(2-benzofuranylcarbonyl)phenyl]thio]phenyl]-4-methyl-1-pentanone-1-(0-acetyloxim e) [product name: IRGACURE (registered trademark) OXE-04, manufactured by BASF SE], 2-(dimethylamino)-2-[(4-methylphenyl)methyl]-1-[4-(4-morpholinyl)phenyl]-1-butanone [product name: IRGACURE (registered trademark) 379EG, manufactured by BASF SE], 2-methyl-1-(4-methylthiophenyl)-2-morpholinopropan-1-one [product name: IRGACURE (registered trademark) 907, manufactured by BASF SE], 2-hydroxy-1-{4-[4-(2-hydroxy-2-methyl-propionyl)benzyl]phenyl}-2-methylpropan-1-one [product name: IRGACURE (registered trademark) 127, manufactured by BASF SE], 2-benzyl-2-dimethylamino-1-(4-morpholinophenyl)-butanone-1 [product name: IRGACURE (registered trademark) 369, manufactured by BASF SE], 2-hydroxy-2-methyl-1-phenyl-propan-1-one [product name: IRGACURE (registered trademark) 1173, manufactured by BASF SE], 1-hydroxy cyclohexyl phenyl ketone [product name: IRGACURE (registered trademark) 184, manufactured by BASF SE], 2,2-dimethoxy-1,2-diphenylethan-1-one (product name: IRGACURE 651, manufactured by BASF SE], an oxime ester-based compound [product name: Lunar (registered trademark) 6, manufactured by DKSH Management Ltd.], 1-[4-(phenylthio)phenyl]-3-cyclopentylpropan-1,2-dione-2-(O-benzoyloxime) (product name: TR-PBG-305, manufactured by TRONLY), 1,2-propanedione, 3-cyclohexyl-1-[9-ethyl-6-(2-furanylcarbonyl)-9H-carbazole-3-yl]-, 2-(O-acetyloxime) (product name: TR-PBG-326, manufactured by TRONLY), 3-cyclohexyl-1-(6-(2-(benzoyloxyimino)hexanoyl)-9-ethyl-9H-carbazole-3-yl)-propan-1,2-dio ne-2-(O-benzoyloxime) (product name: TR-PBG-391, manufactured by TRONLY), and APi-307 (1-(biphenyl-4-yl)-2-methyl-2-morpholinopropan-1-one, manufactured by Shenzhen UV-ChemTech Co., Ltd.).

The photosensitive composition layer may include only one kind of photopolymerization initiator, or may include two or more kinds of photopolymerization initiators.

A content of the photopolymerization initiators is preferably 0.1% by mass or more, and more preferably 0.5% by mass or more with respect to the total mass of the photosensitive composition layer. In addition, the upper limit of the content of the photopolymerization initiator is preferably 10% by mass or less, and more preferably 5% by mass or less with respect to the total mass of the photosensitive composition layer.

[Alkali-Soluble Resin]

The photosensitive composition layer includes an alkali-soluble resin. Since the photosensitive composition layer includes the alkali-soluble resin, the solubility of the photosensitive composition layer (non-exposed portion) in a developer is improved.

In the present disclosure, “alkali-soluble” means that a dissolution rate obtained by the following method is 0.01 μm/sec or more.

A propylene glycol monomethyl ether acetate solution in which a concentration of a target compound (for example, a resin) is 25% by mass is applied to a glass substrate, and then heated in an oven at 100° C. for 3 minutes to form a coating film (thickness of 2.0 μm) of the target compound. The above-described coating film is immersed in a 1% by mass aqueous solution of sodium carbonate (liquid temperature of 30° C.), thereby obtaining the dissolution rate (μm/sec) of the above-described coating film.

In a case where the target compound is not dissolved in propylene glycol monomethyl ether acetate, the target compound is dissolved in an organic solvent other than propylene glycol monomethyl ether acetate (for example, tetrahydrofuran, toluene, or ethanol), which has a boiling point of lower than 200° C.

The alkali-soluble resin preferably includes a structural unit derived from a vinylbenzene derivative, a structural unit having a radically polymerizable group, and a structural unit having an acid group.

(Structural Unit Derived from Vinylbenzene Derivative)

As the structural unit derived from a vinylbenzene derivative (hereinafter, also referred to as a “vinylbenzene derivative unit”), a unit represented by Formula (1) (hereinafter, also referred to as a “unit (1)”) is preferable.

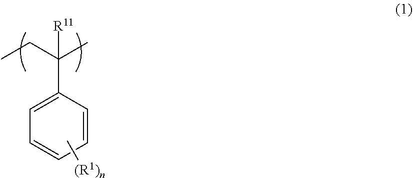

In Formula (1), n represents an integer of 0 to 5. In Formula (1), R¹ represents a substituent. In a case where n is 2 or more, two R¹'s may be bonded to each other to form a fused-ring structure. In a case where n is 2 or more, R¹'s may be the same or different from each other.

As the substituent represented by R¹, a halogen atom, an alkyl group, an aryl group, an alkoxy group, or a hydroxyl group is preferable.

As the halogen atom which is one of the preferred aspects of R¹, a fluorine atom, a chlorine atom, a bromine atom, or an iodine atom is preferable, and a fluorine atom, a chlorine atom, or a bromine atom is more preferable.

The number of carbon atoms in the alkyl group which is one of the preferred aspects of R¹ is preferably 1 to 20, more preferably 1 to 12, still more preferably 1 to 6, even more preferably 1 to 3, particularly preferably 1 or 2, and most preferably 1.

The number of carbon atoms in the aryl group which is one of the preferred aspects of R¹ is preferably 6 to 20, more preferably 6 to 12, still more preferably 6 to 10, and particularly preferably 6.

The number of carbon atoms in the alkoxy group which is one of the preferred aspects of R¹ is preferably 1 to 20, more preferably 1 to 12, still more preferably 1 to 6, even more preferably 1 to 3, particularly preferably 1 or 2, and most preferably 1.

R¹¹ represents a hydrogen atom or a methyl group.

In Formula (1), as n, an integer of 0 to 2 is particularly preferable.

In Formula (1), as the fused-ring structure which can be formed by bonding two R's to each other in a case where n is 2, a naphthalene ring structure or an anthracene ring structure is preferable.

Examples of a monomer for forming the vinylbenzene derivative unit include styrene, 1-vinylnaphthalene, 2-vinylnaphthalene, vinylbiphenyl, vinylanthracene, 4-hydroxystyrene, 4-bromostyrene, 4-methoxystyrene, and a-methylstyrene, and styrene is particularly preferable.

From the viewpoint that the effects of the present invention are more excellent, a content of the vinylbenzene derivative unit is preferably 30% by mass or more, more preferably 40% by mass or more, and still more preferably 45% by mass or more with respect to the total amount of all structural units included in the alkali-soluble resin.

The upper limit value of the content of the vinylbenzene derivative unit is preferably 70% by mass or less, more preferably 60% by mass or less, and still more preferably 50% by mass or less.

The alkali-soluble resin may include only one kind of vinylbenzene derivative unit, or may include two or more kinds of vinylbenzene derivative units.

In the present disclosure, in a case where the content of “structural unit” is specified in % by mass, the “structural unit” is synonymous with “monomer unit” unless otherwise specified. In addition, in the present disclosure, in a case where a resin or polymer has two or more specific structural units, the content of the specific structural units indicates the total content of the two or more specific structural units unless otherwise specified.

(Structural Unit having Radically Polymerizable Group)

In the structural unit having a radically polymerizable group (hereinafter, also referred to as a “radically polymerizable group-containing unit”), as the radically polymerizable group, a group having an ethylenic double bond (hereinafter, also referred to as an “ethylenically unsaturated group”) is preferable, and a (meth)acryloyl group is more preferable.

As the radically polymerizable group-containing unit, a unit represented by Formula (2) (hereinafter, also referred to as a “unit (2)”) is preferable.

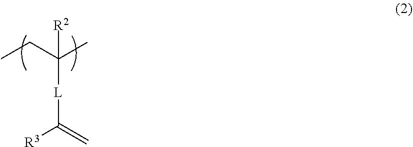

In Formula (2), R² and R³ each independently represent a hydrogen atom or an alkyl group, and L represents a divalent linking group.

The number of carbon atoms in the alkyl group represented by each of R² and R³ is preferably 1 to 3, more preferably 1 or 2, and still more preferably 1.

As the divalent linking group represented by L, one group selected from the group consisting of a carbonyl group (that is, a —C(═O)— group), an oxygen atom (that is, a —O— group), an alkylene group, and an arylene group or a group formed by linking two or more groups selected from the group is preferable.

Each of the alkylene group and the arylene group may be substituted with a substituent (for example, a hydroxyl group other than a primary hydroxyl group, a halogen atom, or the like).

The divalent linking group represented by L may have a branched structure.

The number of carbon atoms in the divalent linking group represented by L is preferably 1 to 30, more preferably 1 to 20, and still more preferably 2 to 10.

As the divalent linking group represented by L, the following groups are particularly preferable.

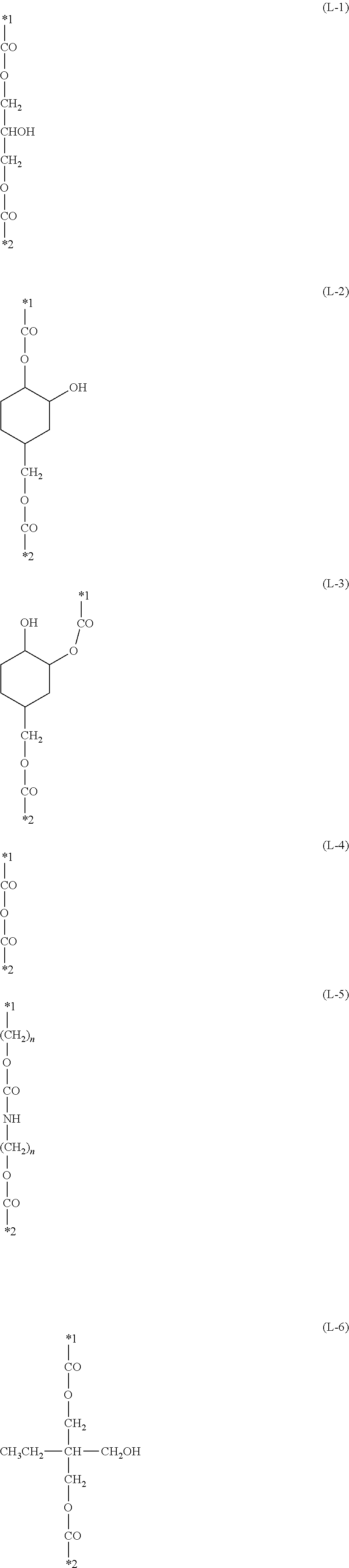

In each of the above groups, *1 represents a bonding position with a carbon atom included in the main chain of Formula (2), and *2 represents a bonding position with a carbon atom forming a double bond in Formula (2).

In addition, in (L-5), n and m each independently represent an integer of 1 to 6.

Examples of the radically polymerizable group-containing unit include a structural unit in which an epoxy group-containing monomer is added to a (meth)acrylic acid unit and a structural unit in which an isocyanate group-containing monomer is added to a hydroxyl group-containing monomer unit.

As the epoxy group-containing monomer, an epoxy group-containing (meth)acrylate having total carbon atoms of 5 to 24 is preferable, an epoxy group-containing (meth)acrylate having total carbon atoms of 5 to 12 is more preferable, and glycidyl (meth)acrylate or 3,4-epoxycyclohexylmethyl (meth)acrylate is still more preferable.

As a hydroxyl group-containing monomer for forming the hydroxyl group-containing monomer unit, a hydroxyalkyl (meth)acrylate having total carbon atoms of 4 to 24 is preferable, a hydroxyalkyl (meth)acrylate having total carbon atoms of 4 to 12 is more preferable, and hydroxyethyl (meth)acrylate is still more preferable.

Here, the “(meth)acrylic acid unit” means a structural unit derived from (meth)acrylic acid.

Similarly, in the present specification, a term “unit” added immediately after the monomer name (for example, “hydroxyl group-containing monomer unit”) means a structural unit derived from the monomer (for example, the hydroxyl group-containing monomer).

More specific examples of the radically polymerizable group-containing unit include

a structural unit in which glycidyl (meth)acrylate is added to a (meth)acrylic acid unit,

a structural unit in which (meth)acrylic acid is added to a (meth)acrylic acid unit,

a structural unit in which 3,4-epoxycyclohexylmethyl (meth)acrylate is added to a (meth)acrylic acid unit,

a structural unit in which 2-isocyanatoethyl (meth)acrylate is added to a hydroxyethyl (meth)acrylate unit,

a structural unit in which 2-isocyanatoethyl (meth)acrylate is added to a hydroxybutyl (meth)acrylate unit, and

a structural unit in which 2-isocyanatoethyl (meth)acrylate is added to a hydroxystyrene unit.

As the radically polymerizable group-containing unit, a structural unit in which glycidyl (meth)acrylate is added to a (meth)acrylic acid unit or a structural unit in which 3,4-epoxycyclohexylmethyl (meth)acrylate is added to a (meth)acrylic acid unit is still more preferable; and

a structural unit in which glycidyl methacrylate is added to a methacrylic acid unit or a structural unit in which 3,4-epoxycyclohexylmethyl methacrylate is added to a methacrylic acid unit is particularly preferable.

From the viewpoint that the effects of the present invention are more excellent, a content of the radically polymerizable group-containing unit is preferably 20% to 50% by mass, more preferably 25% to 45% by mass, and still more preferably 30% to 40% by mass with respect to the total amount of all structural units included in the alkali-soluble resin.

The alkali-soluble resin may include only one kind of radically polymerizable group-containing unit, or may include two or more kinds of radically polymerizable group-containing units.

(Structural Unit Having Acid Group)

In a case where the alkali-soluble resin includes the structural unit having an acid group (hereinafter, also referred to as an “acid group-containing unit”), the photosensitive composition layer has alkali-soluble property.

Examples of the acid group in the acid group-containing unit include a carboxy group, a sulfonic acid group, a sulfate group, and a phosphoric acid group, and a carboxy group is preferable.

As the acid group-containing unit, a unit represented by Formula (3) (hereinafter, also referred to as a “unit (3)”) is preferable.

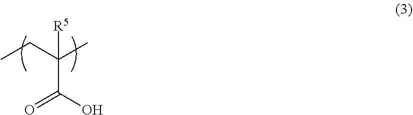

In Formula (3), R⁵ represents a hydrogen atom or an alkyl group.

The number of carbon atoms in the alkyl group represented by R⁵ is preferably 1 to 3, more preferably 1 or 2, and still more preferably 1.

As R⁵, a hydrogen atom or an alkyl group having 1 to 3 carbon atoms is preferable, a hydrogen atom, a methyl group, or an ethyl group is more preferable, and a hydrogen atom or a methyl group is still more preferable.

A monomer for forming the acid group-containing unit, (meth)acrylic acid is particularly preferable.

From the viewpoint that the effects of the present invention are more excellent, a content of the acid group-containing unit is preferably 5% to 30% by mass, more preferably 10% to 25% by mass, and still more preferably 15% to 20% by mass with respect to the total amount of all structural units included in the alkali-soluble resin.

The alkali-soluble resin may include only one kind of acid group-containing unit, or may include two or more kinds of acid group-containing units.

(Other Structural Units)

The alkali-soluble resin may include a structural unit other than the structural units described above.

Examples of other structural units include an alkyl (meth)acrylate structural unit which has a hydroxyl group and does not have a radically polymerizable group and an acid group and an alkyl (meth)acrylate structural unit which does not have a hydroxyl group, a radically polymerizable group, and an acid group.

Examples of a monomer for forming the alkyl (meth)acrylate structural unit which has a hydroxyl group and does not have a radically polymerizable group and an acid group include hydroxyethyl (meth)acrylate and 4-hydroxyethyl (meth)acrylate.

Examples of a monomer for forming the alkyl (meth)acrylate structural unit which does not have a hydroxyl group, a radically polymerizable group, and an acid group include alkyl (meth)acrylates having a monocyclic or polycyclic aliphatic hydrocarbon group (for example, dicyclopentanyl (meth)acrylate, cyclohexyl (meth)acrylate, isobornyl (meth)acrylate, 1-adamantyl (meth)acrylate, and the like) and alkyl (meth)acrylates having a linear or branched aliphatic hydrocarbon group (for example, methyl (meth)acrylate, butyl (meth)acrylate, and the like).

A content of the alkyl (meth)acrylate structural unit which has a hydroxyl group and does not have a radically polymerizable group and an acid group is preferably 0% to 5% by mass and more preferably 1% to 3% by mass with respect to the total amount of all structural units included in the alkali-soluble resin.

A content of the alkyl (meth)acrylate structural unit which does not have a hydroxyl group, a radically polymerizable group, and an acid group is preferably 0% to 5% by mass and more preferably 1% to 3% by mass with respect to the total amount of all structural units included in the alkali-soluble resin.

The alkali-soluble resin may include only one kind of other structural units, or may include two or more kinds of other structural units.

A weight-average molecular weight (Mw) of the alkali-soluble resin is preferably 5,000 or more, more preferably 5,000 to 100,000, and still more preferably 7,000 to 50,000.

From the viewpoint of film hardness, a dispersity (weight-average molecular weight Mw/number-average molecular weight Mn) of the alkali-soluble resin is preferably 1.0 to 3.0 and more preferably 1 to 2.5.

From the viewpoint of developability, an acid value of the alkali-soluble resin is preferably 50 mgKOH/g or more, more preferably 60 mgKOH/g or more, still more preferably 70 mgKOH/g or more, and particularly preferably 80 mgKOH/g or more.

From the viewpoint of suppressing dissolution in a developer, the upper limit of the acid value of the alkali-soluble resin is preferably 200 mgKOH/g or less and more preferably 150 mgKOH/g or less.

As the acid value, a value of the theoretical acid value calculated by the calculation method described in paragraph [0063] of JP2004-149806A, paragraph [0070] of JP2012-211228A, or the like can be used.

The photosensitive composition layer may include only one kind of alkali-soluble resin, or may include two or more kinds of alkali-soluble resins.

The photosensitive composition layer may include residual monomers of each constitutional unit of the above-described alkali-soluble resin.

From the viewpoint of patterning properties and reliability, a content of the residual monomers is preferably 5,000 ppm by mass or less, more preferably 2,000 ppm by mass or less, and still more preferably 500 ppm by mass or less with respect to the total mass of the alkali-soluble resin. The lower limit is not particularly limited, but is preferably 1 ppm by mass or more and more preferably 10 ppm by mass or more.

From the viewpoint of patterning properties and reliability, the residual monomer of each constitutional unit in the alkali-soluble resin is preferably 3,000 ppm by mass or less, more preferably 600 ppm by mass or less, and still more preferably 100 ppm by mass or less with respect to the total mass of the photosensitive composition layer. The lower limit is not particularly limited, but is preferably 0.1 ppm by mass or more, and more preferably 1 ppm by mass or more.

It is preferable that an amount of residual monomers of the monomers in a case of synthesizing the alkali-soluble resin by a polymer reaction is also within the range. For example, in a case where glycidyl acrylate is reacted with a carboxylic acid side chain to synthesize the alkali-soluble resin, a content of glycidyl acrylate is preferably within the range.

The amount of the residual monomers can be measured by a known method such as liquid chromatography and gas chromatography.

From the viewpoint of developability, a content of the alkali-soluble resin is preferably 10% to 90% by mass, more preferably 20% to 80% by mass, and still more preferably 25% to 70% by mass with respect to the total mass of the photosensitive composition layer.

[First Blocked Isocyanate Compound]

The photosensitive composition layer includes a first blocked isocyanate compound.

The blocked isocyanate compound refers to a “compound having a structure in which the isocyanate group of isocyanate is protected (so-called masked) with a blocking agent”. In the present specification, a term “blocked isocyanate compound” includes not only the “first blocked isocyanate compound” but also a “second blocked isocyanate compound” described later. In addition, a structure in which an isocyanate group is protected with a blocking agent may be referred to as the “blocked isocyanate group”.

From the viewpoint that the effects of the present invention are more excellent, an NCO value of the first blocked isocyanate compound is 4.5 mmol/g or more, preferably 5.0 mmol/g or more and more preferably 5.3 mmol/g or more.

From the viewpoint that the effects of the present invention are more excellent, the upper limit value of the NCO value of the first blocked isocyanate compound is preferably 8.0 mmol/g or less, more preferably 6.0 mmol/g or less, still more preferably less than 5.8 mmol/g, and particularly preferably 5.7 mmol/g or less.

The NCO value of the blocked isocyanate compound in the present invention means the number of moles of isocyanate groups included in 1 g of the blocked isocyanate compound, and is a value calculated from the structural formula of the blocked isocyanate compound.

A dissociation temperature of the first blocked isocyanate compound is preferably 100° C. to 160° C., and more preferably 110° C. to 150° C.

In the present specification, the “dissociation temperature of the blocked isocyanate compound” means a temperature at an endothermic peak accompanied with a deprotection reaction of the blocked isocyanate compound, in a case where the measurement is performed by differential scanning calorimetry (DSC) analysis using a differential scanning calorimeter. As the differential scanning calorimeter, for example, a differential scanning calorimeter (model: DSC6200) manufactured by Seiko Instruments Inc. can be suitably used. It should be noted that the differential scanning calorimeter is not limited to the differential scanning calorimeter described above.

Examples of the blocking agent having a dissociation temperature of 100° C. to 160° C. include active methylene compounds [diester malonates (such as dimethyl malonate, diethyl malonate, di-n-butyl malonate, and di-2-ethylhexyl malonate)], and oxime compounds (compound having a structure represented by —C(═N—OH)— in a molecule, such as formaldoxime, acetoaldoxime, acetoxime, methyl ethyl ketoxime, and cyclohexanone oxime). Among these, from the viewpoint of storage stability, an oxime compound is preferable as the blocking agent having a dissociation temperature of 100° C. to 160° C.

From the viewpoint that the effects of the present invention are more excellent, the first blocked isocyanate compound preferably has a ring structure. Examples of the ring structure include an aliphatic hydrocarbon ring, an aromatic hydrocarbon ring, and a heterocyclic ring, and from the viewpoint that the effects of the present invention are more excellent, an aliphatic hydrocarbon ring or an aromatic hydrocarbon ring is preferable, and an aliphatic hydrocarbon ring is more preferable.

Specific examples of the aliphatic hydrocarbon ring include a cyclopentane ring and a cyclohexane ring, and among these, a cyclohexane ring is preferable.

Specific examples of the aromatic hydrocarbon ring include a benzene ring and a naphthalene ring, and among these, a benzene ring is preferable.

Specific examples of the heterocyclic ring include an isocyanurate ring.

In a case where the first blocked isocyanate compound has a ring structure, from the viewpoint that the effects of the present invention are more excellent, the number of rings is preferably 1 or 2 and more preferably 1. In a case where the first blocked isocyanate compound includes a fused ring, the number of rings constituting the fused ring is counted, for example, the number of rings in the naphthalene ring is counted as 2.

From the viewpoint that the strength of the formed pattern is excellent and the effects of the present invention are more excellent, the number of blocked isocyanate groups in the first blocked isocyanate compound is preferably 2 to 5, more preferably 2 or 3, and still more preferably 2.

From the viewpoint that the effects of the present invention are more excellent, the first blocked isocyanate compound is preferably a blocked isocyanate compound represented by Formula Q.

B¹-A¹-L¹-A²-B² Formula Q

In Formula Q, B¹ and B² each independently represent a blocked isocyanate group. The blocked isocyanate group is not particularly limited, but from the viewpoint that the effects of the present invention are more excellent, a group in which an isocyanate group is blocked with an oxime compound is preferable, and a group in which an isocyanate group is blocked with methyl ethyl ketoxime (specifically, a group represented by *—NH—C(═O)—O—N═C(CH₃)—C₂H₅; * represents a bonding position with A¹ or A²) is more preferable.

B¹ and B² are preferably the same group.

In Formula Q, A¹ and A² each independently represent a single bond or an alkylene group having 1 to 10 carbon atoms, and an alkylene group having 1 to 10 carbon atoms is preferable.

The alkylene group may be linear, branched, or cyclic, and is preferably linear.

The number of carbon atoms in the alkylene group is 1 to 10, and from the viewpoint that the effects of the present invention are more excellent, is preferably 1 to 5, more preferably 1 to 3, and still more preferably 1.

A¹ and A² are preferably the same group.

In Formula Q, L¹ represents a divalent linking group.

Specific examples of the divalent linking group include a divalent hydrocarbon group.

Specific examples of the divalent hydrocarbon group include a divalent saturated hydrocarbon group, a divalent aromatic hydrocarbon group, and a group formed by linking two or more of these groups.

The divalent saturated hydrocarbon group may be linear, branched, or cyclic, and from the viewpoint that the effects of the present invention are more excellent, is preferably cyclic. From the viewpoint that the effects of the present invention are more excellent, the number of carbon atoms in the divalent saturated hydrocarbon group is preferably 4 to 15, more preferably 5 to 10, and still more preferably 5 to 8.

The divalent aromatic hydrocarbon group preferably has 5 to 20 carbon atoms, and examples thereof include a phenylene group. The divalent aromatic hydrocarbon group may have a substituent (for example, an alkyl group).

Among these, as the divalent linking group, a linear, branched, or cyclic divalent saturated hydrocarbon group having 5 to 10 carbon atoms, a group in which a cyclic saturated hydrocarbon group having 5 to 10 carbon atoms is linked to a linear alkylene group having 1 to 3 carbon atoms, a divalent aromatic hydrocarbon group which may have a substituent, or a group in which a divalent aromatic hydrocarbon group is linked to a linear alkylene group having 1 to 3 carbon atoms is preferable, a cyclic divalent saturated hydrocarbon group having 5 to 10 carbon atoms or a phenylene group which may have a substituent is more preferable, a cyclohexylene group or a phenylene group which may have a substituent is still more preferable, and a cyclohexylene group is particularly preferable.

From the viewpoint that the effects of the present invention are more excellent, the blocked isocyanate compound represented by Formula Q is particularly preferably a blocked isocyanate compound represented by Formula QA.

B^{1a}-A^{1a}-L^{1a}-A^{2a}-B^{2a} Formula QA

In Formula QA, B^{1a} and B^{2a} each independently represent a blocked isocyanate group. Suitable aspects of B^{1a} and B^{2a} are the same as those of B¹ and B² in Formula Q.

In Formula QA, A^{1a} and A^{2a} each independently represent a divalent linking group. A suitable aspect of the divalent linking group in A^{1a} and A^{2a} is the same as those of A¹ and A² in Formula Q.

In Formula QA, L^{1a} represents a cyclic divalent saturated hydrocarbon group or a divalent aromatic hydrocarbon group.

The number of carbon atoms in the cyclic divalent saturated hydrocarbon group in L^{1a} is preferably 5 to 10, more preferably 5 to 8, still more preferably 5 or 6, and particularly preferably 6.

A suitable aspect of the divalent aromatic hydrocarbon group in L^{1a} is the same as that of L¹ in Formula Q.

Among these, L^{1a} is preferably a cyclic divalent saturated hydrocarbon group, more preferably a cyclic divalent saturated hydrocarbon group having 5 to 10 carbon atoms, still more preferably a cyclic divalent saturated hydrocarbon group having 5 to 8 carbon atoms, particularly preferably a cyclic divalent saturated hydrocarbon group having 5 or 6 carbon atoms, and most preferably a cyclohexylene group.

In a case where L^{1a} is a cyclohexylene group, the blocked isocyanate compound represented by Formula QA may be an isomer mixture of a cis form and a trans form (hereinafter, also referred to as a “cis-trans isomer mixture”).

A mass ratio of the cis form to the trans form is preferably cis form/trans form=10/90 to 90/10, and more preferably cis form/trans form=40/60 to 60/40.

Specific examples of the first blocked isocyanate compound are shown below, but the first blocked isocyanate compound is not limited thereto.

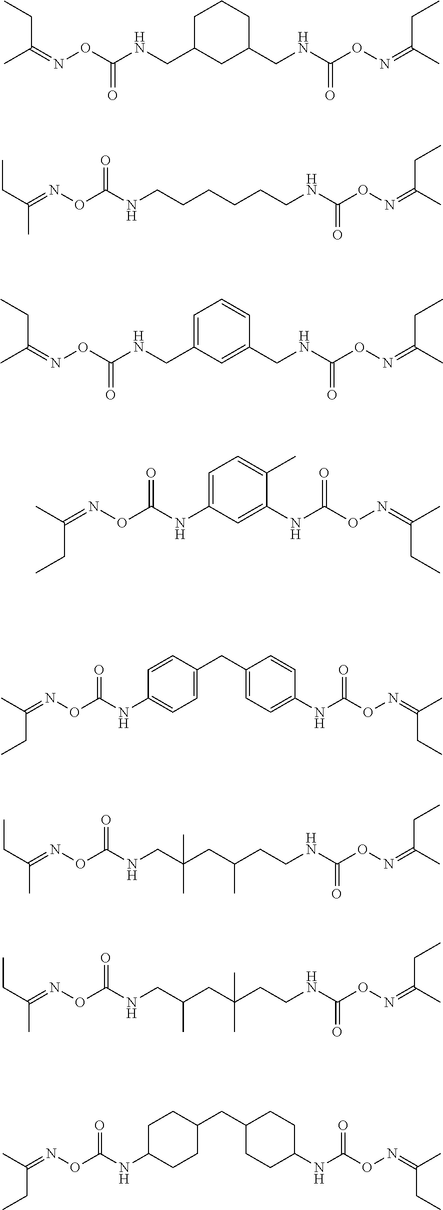

The photosensitive composition layer may include only one kind of first blocked isocyanate compound, or may include two or more kinds of first blocked isocyanate compounds.

From the viewpoint that the effects of the present invention are more excellent, a content of the first blocked isocyanate compound is preferably 1% to 20% by mass, more preferably 2% to 15% by mass, and still more preferably 2.5% to 13% by mass with respect to the total mass of the photosensitive composition layer.

The first blocked isocyanate compound is obtained, for example, by reacting an isocyanate group of a compound having an isocyanate group (for example, a compound in which B¹ and B² in Formula Q described above are isocyanate groups) with the blocking agent.

[Second Blocked Isocyanate Compound]

It is preferable that the photosensitive composition layer further includes a blocked isocyanate compound having an NCO value of less than 4.5 mmol/g (hereinafter, also referred to as a “second blocked isocyanate compound”). As a result, generation of development residue can be suppressed after the photosensitive composition layer is subjected to pattern exposure and development.

The NCO value of the second blocked isocyanate compound is less than 4.5 mmol/g, preferably 3.0 to 4.5 mmol/g, more preferably 3.3 to 4.4 mmol/g, and still more preferably 3.5 to 4.3 mmol/g.

A dissociation temperature of the second blocked isocyanate compound is preferably 100° C. to 160° C. and more preferably 110° C. to 150° C.

Specific examples of a blocking agent having a dissociation temperature of 100° C. to 160° C. are as described above.

From the viewpoint of improvement of brittleness of a film, improvement of adhesive force onto the object to be transferred, or the like, the second blocked isocyanate compound preferably has an isocyanurate structure. The blocked isocyanate compound having an isocyanurate structure can be obtained, for example, by isocyanurate-forming and protecting hexamethylene diisocyanate.

From the viewpoint that it is easier to make the dissociation temperature in a preferred range and to reduce the development residue than a compound not having an oxime structure, as the blocked isocyanate compound having an isocyanurate structure, a compound having an oxime structure, in which an oxime compound is used as the blocking agent, is preferable.

From the viewpoint of strength of a pattern to be formed, the second blocked isocyanate compound may have a polymerizable group. As the polymerizable group, a radically polymerizable group is preferable.

Examples of the polymerizable group include a (meth)acryloxy group, a (meth)acrylamide group, an ethylenically unsaturated group such as styryl group, and an epoxy group such as a glycidyl group. Among these, as the polymerizable group, from the viewpoint of surface shape of the surface of the pattern to be obtained, a development speed, and reactivity, an ethylenically unsaturated group is preferable, and a (meth)acryloxy group is more preferable.

Specific examples of the second blocked isocyanate compound are shown below, but the second blocked isocyanate compound is not limited thereto.

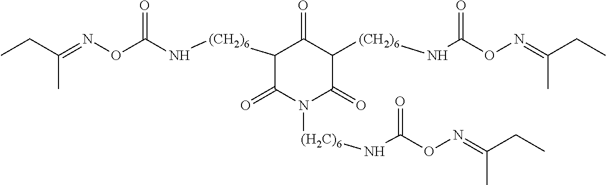

As the second blocked isocyanate compound, a commercially available product can be used. Examples of the commercially available product of the blocked isocyanate compound include KARENZ (registered trademark) AOI-BM, KARENZ (registered trademark) MOI-BM, KARENZ (registered trademark) AOI-BP, KARENZ (registered trademark) MOI-BP, and the like [all manufactured by SHOWA DENKO K.K.], and block-type DURANATE series [for example, DURANATE (registered trademark) TPA-B80E, manufactured by Asahi Kasei Corporation].

The photosensitive composition layer may include only one kind of second blocked isocyanate compound, or may include two or more kinds of second blocked isocyanate compounds.

In a case where the photosensitive composition layer includes the second blocked isocyanate compound, from the viewpoint that the generation of development residue can be further reduced, a content of the second blocked isocyanate compound is preferably 5% to 20% by mass, more preferably 7% to 17% by mass, and still more preferably 10% to 15% by mass with respect to the total mass of the photosensitive composition layer.

In a case where the photosensitive composition layer includes the second blocked isocyanate compound, from the viewpoint of bending resistance, a mass ratio (first blocked isocyanate compound/second blocked isocyanate compound) of the content of the first blocked isocyanate compound to the content of the second blocked isocyanate compound is preferably 0.1 to 1.5, more preferably 0.2 to 1.0, and still more preferably 0.2 to 0.8.

[Polymer Including Structural Unit Having Carboxylic Acid Anhydride Structure]

The photosensitive composition layer may further include, as the binder, a polymer (hereinafter also referred to as a “polymer B”) including a structural unit having a carboxylic acid anhydride structure. In a case where the photosensitive composition layer includes the polymer B, the developability and the hardness after curing can be improved.

The carboxylic acid anhydride structure may be either a chain carboxylic acid anhydride structure or a cyclic carboxylic acid anhydride structure, and a cyclic carboxylic acid anhydride structure is preferable.

The ring of the cyclic carboxylic acid anhydride structure is preferably a 5- to 7-membered ring, more preferably a 5-membered ring or a 6-membered ring, and still more preferably a 5-membered ring.

The structural unit having a carboxylic acid anhydride structure is preferably a structural unit including a divalent group obtained by removing two hydrogen atoms from a compound represented by Formula P-1 in a main chain, or a structural unit in which a monovalent group obtained by removing one hydrogen atom from a compound represented by Formula P-1 is bonded to the main chain directly or through a divalent linking group.

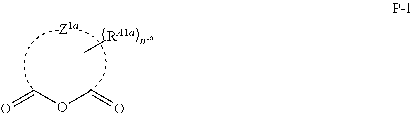

In Formula P-1, R^{A1a} represents a substituent, n^{1a} pieces of R^{A1a}s may be the same or different, Z^{1a} represents a divalent group forming a ring including —C(═O)—O—C(═O)—, and n^{1a} represents an integer of 0 or more.

Examples of the substituent represented by R^{A1a} include an alkyl group.

Z^{1a} is preferably an alkylene group having 2 to 4 carbon atoms, more preferably an alkylene group having 2 or 3 carbon atoms, and still more preferably an alkylene group having 2 carbon atoms.

n^{1a} represents an integer of 0 or more. In a case where Z^{1a} represents an alkylene group having 2 to 4 carbon atoms, n^{1a} is preferably an integer of 0 to 4, more preferably an integer of 0 to 2, and still more preferably 0.

In a case where n^{1a} represents an integer of 2 or more, a plurality of R^{A1a}s may be the same or different from each other. In addition, the plurality of R^{A1a}'s may be bonded to each other to form a ring, but it is preferable that they are not bonded to each other to form a ring.

As the structural unit having a carboxylic acid anhydride structure, a structural unit derived from an unsaturated carboxylic acid anhydride is preferable, a structural unit derived from an unsaturated cyclic carboxylic acid anhydride is more preferable, a structural unit derived from an unsaturated aliphatic carboxylic acid anhydride is still more preferable, a structural unit derived from maleic acid anhydride or itaconic acid anhydride is particularly preferable, and a structural unit derived from maleic acid anhydride is most preferable.

The polymer B may have only one kind of structural unit having a carboxylic acid anhydride structure, or two or more kinds thereof.

A content of the structural unit having a carboxylic acid anhydride structure is preferably 0% to 60% by mole, more preferably 5% to 40% by mole, and still more preferably 10% to 35% by mole with respect to the total amount of the polymer B.

The photosensitive composition layer may include only one kind of polymer B, or may include two or more kinds of polymers B.

From the viewpoint of patterning properties and reliability, a content of the residual monomer of each structural unit of the polymer B in the photosensitive composition layer is preferably 1000 ppm by mass or less, more preferably 500 ppm by mass or less, and still more preferably 100 ppm by mass or less with respect to the total mass of the polymer B. The lower limit is not particularly limited, but is preferably 0.1 ppm by mass or more and more preferably 1 ppm by mass or more.

In a case where the photosensitive composition layer includes the polymer B, from the viewpoint of the developability and the hardness after curing, a content of the polymer B is preferably 0.1% to 30% by mass, more preferably 0.2% to 20% by mass, still more preferably 0.5% to 20% by mass, and particularly preferably 1% to 20% by mass with respect to the total mass of the photosensitive composition layer.

[Heterocyclic Compound]

It is preferable that the photosensitive composition layer includes a heterocyclic compound.

A heterocyclic ring included in the heterocyclic compound may be either a monocyclic or polycyclic heterocyclic ring.

Examples of a heteroatom included in the heterocyclic compound include an oxygen atom, a nitrogen atom, and a sulfur atom. The heterocyclic compound preferably has at least one atom selected from the group consisting of a nitrogen atom, an oxygen atom, and a sulfur atom, and more preferably has a nitrogen atom.

Examples of the heterocyclic compound include a triazole compound, a benzotriazole compound, a tetrazole compound, a thiadiazole compound, a triazine compound, a rhodanine compound, a thiazole compound, a benzothiazole compound, a benzoimidazole compound, a benzoxazole compound, and a pyrimidine compound (for example, isonicotinamide).

Among these, as the heterocyclic compound, at least one compound selected from the group consisting of a triazole compound, a benzotriazole compound, a tetrazole compound, a thiadiazole compound, a triazine compound, a rhodanine compound, a thiazole compound, a benzimidazole compounds, and a benzoxazole compound is preferable, and at least one compound selected from the group consisting of a triazole compound, a benzotriazole compound, a tetrazole compound, a thiadiazole compound, a thiazole compound, a benzothiazole compound, a benzoimidazole compound, and a benzoxazole compound is more preferable.

Preferred specific examples of the heterocyclic compound are shown below. The following compounds can be exemplified as a triazole compound and a benzotriazole compound.

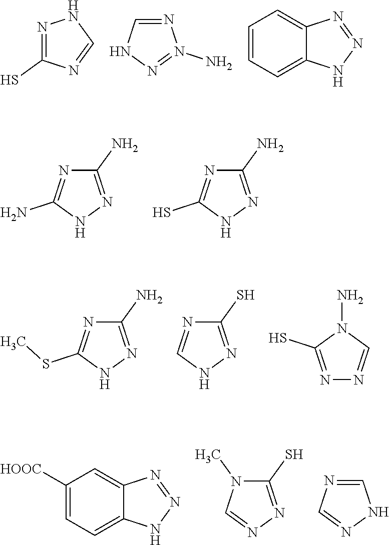

Examples of the tetrazole compound include the following compounds.

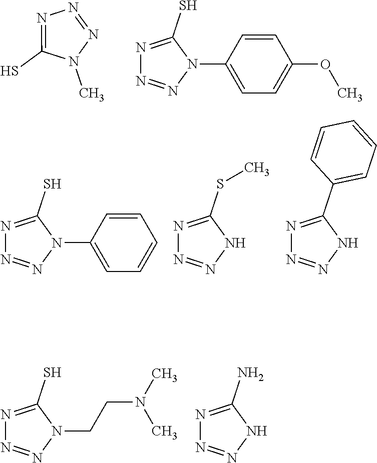

Examples of the thiadiazole compound include the following compounds.

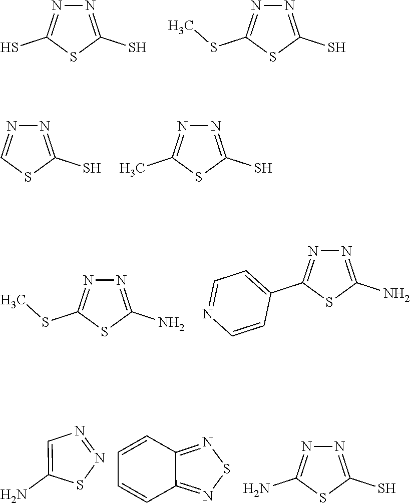

Examples of the triazine compound include the following compounds.

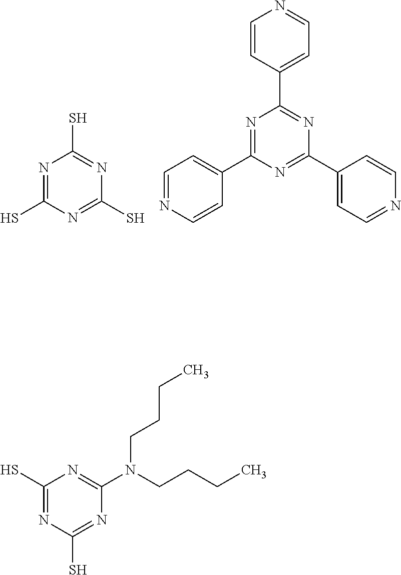

The following compounds can be exemplified as a rhodanine compound.

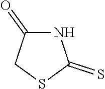

Examples of the thiazole compound include the following compounds.

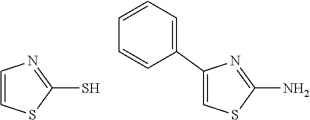 [SMILES: Sc1nccs1 ; Nc1nc(-c2ccccc2)cs1]

Examples of the benzothiazole compound include the following compounds.

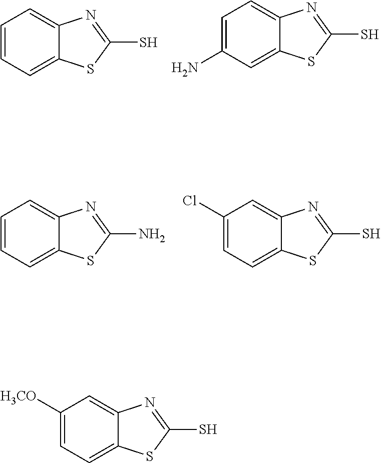 [SMILES: Sc1nc2ccccc2s1 ; Nc1ccc2nc(S)sc2c1 ; Nc1nc2ccccc2s1 ; Sc1nc2cc(Cl)ccc2s1 ; COc1ccc2sc(S)nc2c1]

Examples of the benzoimidazole compound include the following compounds.

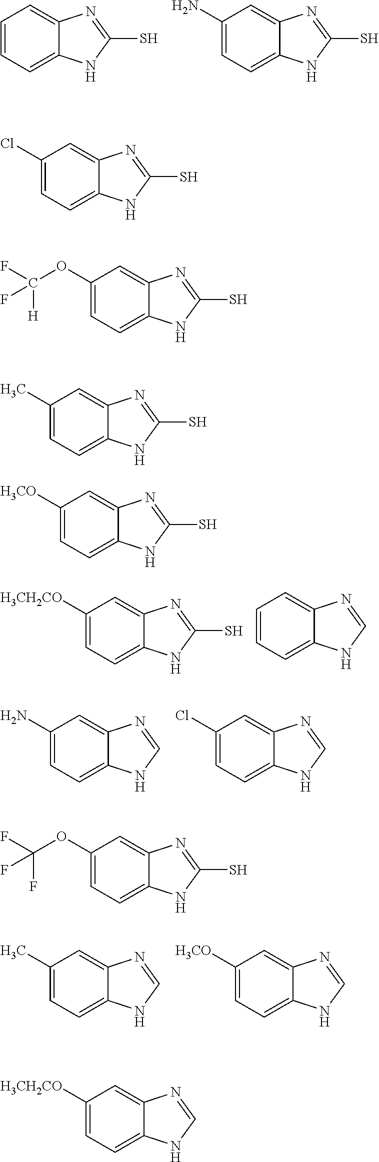

Examples of the benzoxazole compound include the following compounds.

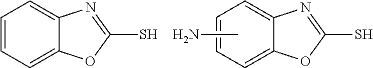

The photosensitive composition layer may include only one kind of heterocyclic compound, or may include two or more kinds of heterocyclic compounds.

In a case where the photosensitive composition layer includes the heterocyclic compound, a content of the heterocyclic compound is preferably 0.01% to 20% by mass, more preferably 0.1% to 10% by mass, still more preferably 0.3% to 8% by mass, and particularly preferably 0.5% to 5% by mass with respect to the total mass of the photosensitive composition layer.

[Aliphatic Thiol Compound]

It is preferable that the photosensitive composition layer includes an aliphatic thiol compound.

In a case where the photosensitive composition layer includes the aliphatic thiol compound, the aliphatic thiol compound undergoes an ene-thiol reaction with a radically polymerizable compound having an ethylenically unsaturated group, so that a film to be formed is suppressed from being cured and shrunk and the stress is relieved.

As the aliphatic thiol compound, a monofunctional aliphatic thiol compound or a polyfunctional aliphatic thiol compound (that is, a bi- or higher functional aliphatic thiol compound) is preferable.

Among these, as the aliphatic thiol compound, for example, from the viewpoint of adhesiveness (in particular, adhesiveness after exposure) of the pattern to be formed, a polyfunctional aliphatic thiol compound is preferable.

In the present disclosure, the “polyfunctional aliphatic thiol compound” refers to an aliphatic compound having two or more thiol groups (also referred to as “mercapto groups”) in a molecule.

As the polyfunctional aliphatic thiol compound, a low-molecular-weight compound having a molecular weight of 100 or more is preferable. Specifically, a molecular weight of the polyfunctional aliphatic thiol compound is more preferably 100 to 1,500 and still more preferably 150 to 1,000.

From the viewpoint of the adhesiveness of the pattern to be formed, the number of functional groups in the polyfunctional aliphatic thiol compound is, for example, preferably 2 to 10, more preferably 2 to 8, and still more preferably 2 to 6.

Examples of the polyfunctional aliphatic thiol compound include trimethylolpropane tris(3-mercaptobutyrate), 1,4-bis(3-mercaptobutyryloxy)butane, pentaerythritol tetrakis(3-mercaptobutyrate), 1,3,5-tris(3-mercaptobutyryloxyethyl)-1,3,5-triazine-2,4,6(1H,3H,5H)-trione, trimethylolethane tris(3-mercaptobutyrate), tris[(3-mercaptopropionyloxy)ethyl] isocyanurate, trimethylolpropane tris(3-mercaptopropionate), pentaerythritol tetrakis(3-mercaptopropionate), tetraethylene glycol bis(3-mercaptopropionate), dipentaerythritol hexakis(3-mercaptopropionate), ethylene glycol bisthiopropionate, 1,4-bis(3-mercaptobutyryloxy)butane, 1,2-ethanedithiol, 1,3-propanedithiol, 1,6-hexamethylenedithiol, 2,2′-(ethylenedithio)diethanethiol, meso-2,3-dimercaptosuccinic acid, and di(mercaptoethyl) ether.

Among these, the polyfunctional aliphatic thiol compound is preferably at least one compound selected from the group consisting of trimethylolpropane tris(3-mercaptobutyrate), 1,4-bis(3-mercaptobutyryloxy)butane, and 1,3,5-tris(3-mercaptobutyryloxyethyl)-1,3,5-triazine-2,4,6(1H,3H,5H)-trione.

Examples of the monofunctional aliphatic thiol compound include 1-octanethiol, 1-dodecanethiol, β-mercaptopropionic acid, methyl-3-mercaptopropionate, 2-ethylhexyl-3-mercaptopropionate, n-octyl-3-mercaptopropionate, methoxybutyl-3-mercaptopropionate, and stearyl-3-mercaptopropionate.

The photosensitive composition layer may include only one kind of aliphatic thiol compound, or may contain two or more kinds of aliphatic thiol compounds.

In a case where the photosensitive composition layer includes the aliphatic thiol compound, a content of the aliphatic thiol compound is preferably 5% by mass or more, more preferably 5% to 50% by mass, still more preferably 5% to 30% by mass, and particularly preferably 8% to 20% by mass with respect to the total mass of the photosensitive composition layer.

[Surfactant]

It is preferable that the photosensitive composition layer includes a surfactant.

Examples of the surfactant include the surfactants described in paragraph [0017] of JP4502784B and paragraphs [0060] to [0071] of JP2009-237362A.

As the surfactant, a nonionic surfactant, a fluorine-based surfactant, or a silicon-based surfactant is preferable.

Examples of a commercially available product of the fluorine-based surfactant include: MEGAFACE F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, EXP, MFS-330, MFS-578, MFS-579, MFS-586, MFS-587, R-41, R-41-LM, R-01, R-40, R-40-LM, RS-43, TF-1956, RS-90, R-94, RS-72-K, and DS-21 (all manufactured by DIC Corporation); FLUORAD FC430, FC431, and FC171 (all manufactured by Sumitomo 3M Ltd.); SURFLON S-382, SC-101, SC-103, SC-104, SC-105, SC-1068, SC-381, SC-383, 5-393, and KH-40 (all manufactured by Asahi Glass Co., Ltd.); PolyFox PF636, PF656, PF6320, PF6520, and PF7002 (all manufactured by OMNOVA Solutions Inc.); and FTERGENT 710FL, 710FM, 610FM, 601AD, 601ADH2, 602A, 215M, 245F, 251, 212M, 250, 209F, 222F, 208Q 710LA, 710FS, 730LM, 650AC, 681, and 683 (all manufactured by NEOS Co., Ltd.).

In addition, as the fluorine-based surfactant, an acrylic compound which has a molecular structure having a functional group containing a fluorine atom and in which the functional group containing a fluorine atom is broken to volatilize a fluorine atom by applying heat to the molecular structure can also be suitably used. Examples of such a fluorine-based surfactant include MEGAFACE DS series manufactured by DIC Corporation (The Chemical Daily (Feb. 22, 2016) and Nikkei Business Daily (Feb. 23, 2016)), for example, MEGAFACE DS-21.

In addition, as the fluorine-based surfactant, a polymer of a fluorine atom-containing vinyl ether compound having a fluorinated alkyl group or a fluorinated alkylene ether group, and a hydrophilic vinyl ether compound can also be preferably used.

In addition, a block polymer can also be used as the fluorine-based surfactant.

As the fluorine-based surfactant, a fluorine-containing polymer compound including a constitutional unit derived from a (meth)acrylate compound having a fluorine atom and a constitutional unit derived from a (meth)acrylate compound having 2 or more (preferably 5 or more) alkyleneoxy groups (preferably ethyleneoxy groups or propyleneoxy groups) can also be preferably used.

In addition, as the fluorine-based surfactant, a fluorine-containing polymer having an ethylenically unsaturated bond-containing group at a side chain can also be used. Examples thereof include MEGAFACE RS-101, RS-102, RS-718K, and RS-72-K (all manufactured by DIC Corporation).

As the fluorine-based surfactant, from the viewpoint of improving environmental suitability, a surfactant derived from a substitute material for a compound having a linear perfluoroalkyl group having 7 or more carbon atoms, such as perfluorooctanoic acid (PFOA) and perfluorooctanesulfonic acid (PFOS), is preferable.

Examples of the nonionic surfactant include glycerol, trimethylolpropane, trimethylolethane, an ethoxylate and propoxylate thereof (for example, glycerol propoxylate or glycerol ethoxylate), polyoxyethylene lauryl ether, polyoxyethylene stearyl ether, polyoxyethylene oleyl ether, polyoxyethylene octylphenyl ether, polyoxyethylene nonylphenyl ether, polyethylene glycol dilaurate, polyethylene glycol distearate, sorbitan fatty acid esters, PLURONIC L10, L31, L61, L62, 10R5, 17R2, and 25R2 (all manufactured by BASF SE), TETRONIC 304, 701, 704, 901, 904, and 150R1 (all manufactured by BASF SE), SOLSPERSE 20000 (manufactured by Lubrizol Corporation), NCW-101, NCW-1001, and NCW-1002 (all manufactured by FUJIFILM Wako Pure Chemical Corporation), PIONIN D-6112, D-6112-W, and D-6315 (all manufactured by Takemoto Oil&Fat Co., Ltd.), and OLFINE E1010 and SURFYNOL 104, 400, and 440 (all manufactured by Nissin Chemical Co., Ltd.).

Examples of a commercially available product of the silicon-based surfactant include DOWSIL 8032 ADDITIVE, TORAY SILICONE DC3PA, TORAY SILICONE SH7PA, TORAY SILICONE DC11PA, TORAY SILICONE SH21PA, TORAY SILICONE SH28PA, TORAY SILICONE SH29PA, TORAY SILICONE SH30PA, and TORAY SILICONE SH8400 (all manufactured by Dow Corning Toray Co., Ltd.), X-22-4952, X-22-4272, X-22-6266, KF-351A, K354L, KF-355A, KF-945, KF-640, KF-642, KF-643, X-22-6191, X-22-4515, KF-6004, KP-341, KF-6001, and KF-6002 (all manufactured by Shin-Etsu Silicone Co., Ltd.), F-4440, TSF-4300, TSF-4445, TSF-4460, and TSF-4452 (all manufactured by Momentive Performance Materials Co., Ltd.), and BYK307, BYK323, and BYK330 (all manufactured by BYK Chemie).

The photosensitive composition layer may include only one kind of surfactant, or may include two or more kinds of surfactants.

In a case where the photosensitive composition layer includes the surfactant, a content of the surfactant is preferably 0.01% to 3% by mass, more preferably 0.05% to 1% by mass, and still more preferably 0.1% to 0.8% by mass with respect to the total mass of the photosensitive composition layer.

[Hydrogen Donating Compound]

It is preferable that the photosensitive composition layer includes a hydrogen donating compound. The hydrogen donating compound has a function of further improving sensitivity of the photopolymerization initiator to actinic ray, or suppressing inhibition of polymerization of the polymerizable compound by oxygen.

Examples of such a hydrogen donating compound include amines, for example, compounds described in M. R. Sander et al., “Journal of Polymer Society,” Vol. 10, page 3173 (1972), JP1969-020189B (JP-S44-020189B), JP1976-082102A (JP-S51-082102A), JP1977-134692A (JP-S52-134692A), JP1984-138205A (JP-S59-138205A), JP1985-084305A (JP-S60-084305A), JP1987-018537A (JP-S62-018537A), JP1989-033104A (JP-S64-033104A), and Research Disclosure 33825.

Specific examples of the hydrogen donating compound include triethanolamine, p-dimethylaminobenzoic acid ethyl ester, p-formyldimethylaniline, and p-methylthiodimethylaniline.

In addition, examples of the hydrogen donating compound also include an amino acid compound (N-phenylglycine and the like), an organic metal compound described in JP1973-042965B (JP-S48-042965B) (tributyl tin acetate and the like), a hydrogen donor described in JP1980-034414B (JP-S55-034414B), and a sulfur compound described in JP1994-308727A (JP-H6-308727A) (trithiane and the like).

The photosensitive composition layer may include only one kind of hydrogen donating compound, or may include two or more kinds of hydrogen donating compounds.

In a case where the photosensitive composition layer includes the hydrogen donating compound, from the viewpoint of improving a curing rate by balancing the polymerization growth rate and the chain transfer, a content of the hydrogen donating compound is preferably 0.01% to 10% by mass, more preferably 0.03% to 5% by mass, and still more preferably 0.05% to 3% by mass with respect to the total mass of the photosensitive composition layer.

[Other Components]

The photosensitive composition layer may include a component other than the above-described components (hereinafter also referred to as “other components”). Examples of the other components include particles (for example, metal oxide particles) and a colorant.

In addition, examples of the other components include a thermal polymerization inhibitor described in paragraph [0018] of JP4502784B and other additives described in paragraphs [0058] to [0071] of JP2000-310706A.

The photosensitive composition layer may include particles for the purpose of adjusting refractive index, light-transmitting property, and the like. Examples of the particles include metal oxide particles.

Examples of a metal in the metal oxide particles also include semi-metals such as B, Si, Ge, As, Sb, and Te.

From a viewpoint of transparency of a pattern, an average primary particle diameter of the particles is, for example, preferably 1 to 200 nm, and more preferably 3 to 80 nm. The average primary particle diameter of the particles is calculated by measuring particle diameters of 200 random particles using an electron microscope, and arithmetically averaging the measurement results. In a case where the shape of the particle is not a spherical shape, the longest side is set as the particle diameter.

The photosensitive composition layer may include only one kind of particles, or may include two or more kinds of particles. In addition, in a case where the photosensitive composition layer includes the particles, it may include only one kind of particles having different metal types, sizes, and the like, or may include two or more kinds thereof.

It is preferable that the photosensitive composition layer does not include particles, or the content of the particles is more than 0% by mass to 35% by mass or less with respect to the total mass of the photosensitive composition layer; it is more preferable that the photosensitive composition layer does not include particles, or the content of the particles is more than 0% by mass to 10% by mass or less with respect to the total mass of the photosensitive composition layer; it is still more preferable that the photosensitive composition layer does not include particles, or the content of the particles is more than 0% by mass to 5% by mass or less with respect to the total mass of the photosensitive composition layer; it is particularly preferable that the photosensitive composition layer does not include particles, or the content of the particles is more than 0% by mass to 1% by mass or less with respect to the total mass of the photosensitive composition layer; and it is the most preferable that the photosensitive composition layer does not include particles.

The photosensitive composition layer may include a trace amount of a colorant (for example, a pigment and a dye), but for example, from the viewpoint of transparency, it is preferable that the photosensitive composition layer does not substantially include the colorant.

In a case where the photosensitive composition layer includes the colorant, a content of the colorant is preferably less than 1% by mass, and more preferably less than 0.1% by mass with respect to the total mass of the photosensitive composition layer.

[Impurities and the Like]

The photosensitive composition layer may include a predetermined amount of impurities.

Examples of the impurities include sodium, potassium, magnesium, calcium, iron, manganese, copper, aluminum, titanium, chromium, cobalt, nickel, zinc, tin, halogen, and ions of these. Among these, the halide ion, the sodium ion, and the potassium ion are easily mixed as impurities, and thus, the following content is preferable.

A content of the impurities in the photosensitive composition layer is preferably 80 ppm or less, more preferably 10 ppm or less, and still more preferably 2 ppm or less on a mass basis. The content of the impurities in the photosensitive composition layer may be 1 ppb or more or 0.1 ppm or more on a mass basis.

Examples of a method for keeping the impurities in the range include selecting a raw material having a low content of impurities as a raw material for the photosensitive composition layer, preventing the impurities from being mixed in a case of forming the photosensitive composition layer, and washing and removing the impurities. By such a method, the amount of impurities can be kept within the range.

The impurities can be quantified by a known method such as inductively coupled plasma (ICP) emission spectroscopy, atomic absorption spectroscopy, and ion chromatography.

In addition, it is preferable that the content of compounds such as benzene, formaldehyde, trichloroethylene, 1,3-butadiene, carbon tetrachloride, chloroform, N,N-dimethylformamide, N,N-dimethylacetamide, and hexane is low in the photosensitive composition layer. A content of these compounds in the photosensitive composition layer is preferably 100 ppm or less, more preferably 20 ppm or less, and still more preferably 4 ppm or less on a mass basis. The lower limit may be 10 ppb or more or 100 ppb or more on a mass basis. The content of these compounds can be suppressed in the same manner as in the metal as impurities. In addition, the compounds can be quantified by a known measurement method.

From the viewpoint of reliability and a laminating property, the content of water in the photosensitive composition layer is preferably 0.01% to 1.0% by mass, and more preferably 0.05% to 0.5% by mass.

[Thickness of Photosensitive Composition Layer]

From the viewpoint of coatability, the upper limit value of a thickness of the photosensitive composition layer is preferably 20.0 μm or less, more preferably 15.0 μm or less, and still more preferably 12.0 μm or less.

The lower limit value of the thickness of the photosensitive composition layer is preferably 0.05 μm or more, and from the viewpoint that the effects of the present invention are more excellent, more preferably 3.0 μm or more, still more preferably 4.0 μm or more, and particularly preferably 5.0 μm or more.

The thickness of the photosensitive composition layer is obtained as an average value at 5 random points measured by cross-section observation with a scanning electron microscope (SEM).

[Refractive Index of Photosensitive Composition Layer]

A refractive index of the photosensitive composition layer is preferably 1.47 to 1.56, and more preferably 1.49 to 1.54.

[Color of Photosensitive Composition Layer]

The photosensitive composition layer is preferably achromatic. The a* value of the photosensitive composition layer is preferably −1.0 to 1.0, and the b* value of the photosensitive composition layer is preferably −1.0 to 1.0.

The hue of the photosensitive composition layer can be measured using a colorimeter (CR-221, manufactured by Minolta Co., Ltd.).

[NCO Value of Photosensitive Composition Layer]

From the viewpoint that the effects of the present invention are more excellent, the NCO value of the photosensitive composition layer is preferably more than 0.50 mmol/g, more preferably 0.55 mmol/g or more, and still more preferably 0.60 mmol/g or more. From the viewpoint that the effects of the present invention are more excellent, the upper limit value of the NCO value of the photosensitive composition layer is preferably 1.0 mmol/g or less, more preferably less than 0.80 mmol/g, and still more preferably 0.70 mmol/g or less.

The NCO value of the photosensitive composition layer in the present invention means the number of moles of isocyanate groups included in 1 g of the photosensitive composition layer, and is a value calculated from the structural formula of the blocked isocyanate compound.

[Transmittance of Photosensitive Composition Layer]

A visible light transmittance of the photosensitive composition layer at a film thickness of approximately 1.0 μm is preferably 80% or more, more preferably 90% or more, and most preferably 95% or more.

As the visible light transmittance, it is preferable that an average transmittance at a wavelength of 400 nm to 800 nm, the minimum value of the transmittance at a wavelength of 400 nm to 800 nm, and a transmittance at a wavelength of 400 nm all satisfy the above.

Examples of a preferred value of the transmittance include 87%, 92%, and 98%.

The same applies to a transmittance of the cured film of the photosensitive composition layer at a film thickness of approximately 1.0 μm.

[Moisture Permeability of Photosensitive Composition Layer]

From the viewpoint of rust preventive property of electrode or wiring line, and viewpoint of device reliability, a moisture permeability of the pattern obtained by curing the photosensitive composition layer (cured film of the photosensitive composition layer) at a film thickness of 40 μm is preferably 500 g/m²·24 hr or less, more preferably 300 g/m²·24 hr or less, and still more preferably 100 g/m²·24 hr or less.

The moisture permeability is measured with a cured film by curing the photosensitive composition layer by exposing the photosensitive composition layer with an i-line at an exposure amount of 300 mJ/cm² and then performing post-baking at 145° C. for 30 minutes.

The moisture permeability is measured according to a cup method of JIS Z0208. It is preferable that the above-described moisture permeability is as above under any test conditions of temperature 40° C. and humidity 90%, temperature 65° C. and humidity 90%, or temperature 80° C. and humidity 95%.

Examples of a specific preferred numerical value include 80 g/m²·24 hr, 150 g/m²·24 hr, and 220 g/m²·24 hr.

[Dissolution Rate of Photosensitive Composition Layer]

From the viewpoint of suppressing residue during development, a dissolution rate of the photosensitive composition layer in a 1.0% by mass sodium carbonate aqueous solution is preferably 0.01 μm/sec or more, more preferably 0.10 μm/sec or more, and still more preferably 0.20 μm/sec or more.

From the viewpoint of edge shape of the pattern, it is preferable to be 5.0 μm/sec or less, more preferable to be 4.0 μm/sec or less, and still more preferable to be 3.0 μm/sec or less.

Examples of a specific preferred numerical value include 1.8 μm/sec, 1.0 μm/sec, and 0.7 μm/sec.

The dissolution rate of the photosensitive composition layer in a 1.0% by mass sodium carbonate aqueous solution per unit time is measured as follows.

A photosensitive composition layer (within a film thickness of 1.0 to 10 μm) formed on a glass substrate, from which the solvent has been sufficiently removed, is subjected to a shower development with a 1.0% by mass sodium carbonate aqueous solution at 25° C. until the photosensitive composition layer is dissolved completely (however, the maximum time is 2 minutes).

The dissolution rate of the photosensitive composition layer is obtained by dividing the film thickness of the photosensitive composition layer by the time required for the photosensitive composition layer to dissolve completely. In a case where the photosensitive layer is not dissolved completely in 2 minutes, the dissolution rate of the photosensitive layer is calculated in the same manner as above, from the amount of change in film thickness up to 2 minutes.

A dissolution rate of the cured film (within a film thickness of 1.0 to 10 μm) of the photosensitive composition layer in a 1.0% by mass sodium carbonate aqueous solution is preferably 3.0 μm/sec or less, more preferably 2.0 μm/sec or less, still more preferably 1.0 m/sec or less, and most preferably 0.2 μm/sec or less. The cured film of the photosensitive composition layer is a film obtained by exposing the photosensitive composition layer with i-rays at an exposure amount of 300 mJ/cm².

Examples of a specific preferred numerical value include 0.8 μm/sec, 0.2 μm/sec, and 0.001 μm/sec.

For development, a shower nozzle of 1/4 MINJJX030PP manufactured by H.IKEUCHI Co., Ltd. is used, and a spraying pressure of the shower is set to 0.08 MPa. Under the above-described conditions, a shower flow rate per unit time is set to 1,800 mL/min.

[Swelling Ratio of Photosensitive Composition Layer]

From the viewpoint of improving pattern forming properties, a swelling ratio of the photosensitive composition layer after exposure with respect to a 1.0% by mass sodium carbonate aqueous solution is preferably 100% or less, more preferably 50% or less, and still more preferably 30% or less.

The swelling ratio of the photosensitive composition layer after exposure with respect to a 1.0% by mass sodium carbonate aqueous solution is measured as follows.

A photosensitive composition layer (within a film thickness of 1.0 to 10 μm) formed on a glass substrate, from which the solvent has been sufficiently removed, is exposed at an exposure amount of 500 mJ/cm² (i-ray measurement) with an ultra-high pressure mercury lamp. The glass substrate is immersed in a 1.0% by mass sodium carbonate aqueous solution at 25° C., and the film thickness is measured after 30 seconds. Then, an increased proportion of the film thickness after immersion to the film thickness before immersion is calculated. Examples of a specific preferred numerical value include 4%, 13%, and 25%.

[Foreign Substance in Photosensitive Composition Layer]

From the viewpoint of pattern forming properties, the number of foreign substances having a diameter of 1.0 μm or more in the photosensitive composition layer is preferably 10 pieces/mm² or less, and more preferably 5 pieces/mm² or less.

The number of foreign substances is measured as follows.

Any 5 regions (1 mm×1 mm) on a surface of the photosensitive composition layer are visually observed from a normal direction of the surface of the photosensitive composition layer with an optical microscope, the number of foreign substances having a diameter of 1.0 am or more in each region is measured, and the values are arithmetically averaged to calculate the number of foreign substances.

Examples of a specific preferred numerical value include 0 pieces/mm², 1 pieces/mm², 4 pieces/mm², and 8 pieces/mm².

[Haze of Dissolved Substance in Photosensitive Composition Layer]

From the viewpoint of suppressing generation of aggregates during development, a haze of a solution obtained by dissolving 1.0 cm³ of the photosensitive composition layer in 1.0 liter of a 1.0% by mass sodium carbonate aqueous solution at 30° C. is preferably 60% or less, more preferably 30% or less, still more preferably 10% or less, and most preferably 1% or less.

The haze is measured as follows.

First, a 1.0% by mass sodium carbonate aqueous solution is prepared, and a liquid temperature is adjusted to 30° C. 1.0 cm³ of the photosensitive composition layer is added to 1.0 L of the sodium carbonate aqueous solution. The solution is stirred at 30° C. for 4 hours, being careful not to mix air bubbles. After stirring, the haze of the solution in which the photosensitive composition layer is dissolved is measured. The haze is measured using a haze meter (product name “NDH4000”, manufactured by Nippon Denshoku Industries Co., Ltd.), a liquid measuring unit, and a liquid measuring cell having an optical path length of 20 mm.

Examples of a specific preferred numerical value include 0.4%, 1.0%, 9%, and 24%.

<Refractive Index-Adjusting Layer>

The first transfer film may have a refractive index-adjusting layer. The position of the refractive index-adjusting layer is not particularly limited, but the refractive index-adjusting layer is preferably disposed in contact with the photosensitive composition layer. Among these, it is preferable that the first transfer film has the temporary support, the photosensitive composition layer, and the refractive index-adjusting layer in this order.

In a case where the first transfer film further has a protective film which will be described later, it is preferable that the first transfer film has the temporary support, the photosensitive composition layer, the refractive index-adjusting layer, and the protective film in this order.

As the refractive index-adjusting layer, a known refractive index-adjusting layer can be adopted. Examples of a material included in the refractive index-adjusting layer include a binder and particles.

Examples of the binder include the alkali-soluble resin described in the section of “Photosensitive Composition Layer” above.

Examples of the particles include zirconium oxide particles (ZrO₂ particles), niobium oxide particles (Nb₂O₅ particles), titanium oxide particles (TiO₂ particles), and silicon dioxide particles (SiO₂ particles).

In addition, the refractive index-adjusting layer preferably includes a metal oxidation inhibitor. In a case where the refractive index-adjusting layer includes a metal oxidation inhibitor, oxidation of metal in contact with the refractive index-adjusting layer can be suppressed.

As the metal oxidation inhibitor, for example, a compound having an aromatic ring including a nitrogen atom in the molecule is preferable. Examples of the metal oxidation inhibitor include imidazole, benzoimidazole, tetrazole, mercaptothiadiazole, and benzotriazole.

A refractive index of the refractive index-adjusting layer is preferably 1.60 or more and more preferably 1.63 or more.

The upper limit of the refractive index of the refractive index-adjusting layer is preferably 2.10 or less and more preferably 1.85 or less.

A thickness of the refractive index-adjusting layer is preferably 500 nm or less, more preferably 110 nm or less, and still more preferably 100 nm or less.

The thickness of the refractive index-adjusting layer is preferably 20 nm or more and more preferably 50 nm or more.

The thickness of the refractive index-adjusting layer is obtained as an average value at 5 random points measured by cross-section observation with a scanning electron microscope (SEM).

<Other Layers>

The first transfer film may include a layer other than the temporary support, the photosensitive composition layer, and the refractive index-adjusting layer described above.

Examples of other layers include a protective film and an antistatic layer.

The first transfer film may have a protective film for protecting the photosensitive composition layer on a surface opposite to the temporary support.

The protective film is preferably a resin film, and a resin film having heat resistance and solvent resistance can be used.

Examples of the protective film include polyolefin films such as a polypropylene film and a polyethylene film. In addition, a resin film composed of the same material as the above-described temporary support may be used as the protective film.

A thickness of the protective film is preferably 1 to 100 μm, more preferably 5 to 50 μm, still more preferably 5 to 40 μm, and particularly preferably 15 to 30 μm. The thickness of the protective film is preferably 1 μm or more from the viewpoint of excellent mechanical hardness, and is preferably 100 μm or less from viewpoint of relatively low cost.

The first transfer film may include an antistatic layer.

In a case where the first transfer film includes an antistatic layer, since it is possible to suppress generation of static electricity in a case of peeling off the film or the like disposed on the antistatic layer, and also to suppress generation of static electricity due to rubbing against equipment, other films, or the like, for example, it is possible to suppress occurrence of defect in an electronic apparatus.

The antistatic layer is preferably disposed between the temporary support and the photosensitive composition layer.

The antistatic layer is a layer having antistatic properties, and includes at least an antistatic agent. The antistatic agent is not particularly limited, and a known antistatic agent can be adopted.

### Second Embodiment of Transfer Film

The transfer film according to a second embodiment of the present invention (hereinafter, also referred to as a “second transfer film”) has a temporary support and a photosensitive composition layer disposed on the temporary support, in which the photosensitive composition layer includes an alkali-soluble resin, a polymerizable compound, a polymerization initiator, and a blocked isocyanate compound, and an NCO value of the photosensitive composition layer is more than 0.50 mmol/g.

A feature point of the second transfer film is that the NCO value of the photosensitive composition layer is more than 0.50 mmol/g.

Here, examples of a method for forming a protective film using the second transfer film include a method in which a substrate having a conductive layer (sensor electrode and lead wire) or the like is brought into contact with the second transfer film to affix the substrate to the second transfer film, and through steps such as pattern exposure of the photosensitive composition layer having the second transfer film, development, and post-baking, a protective film in a patterned shape is formed.

The alkali-soluble resin included in the photosensitive composition layer is required from the viewpoint of developability of the photosensitive composition layer, but the present inventors have found that corrosion of the conductive layer may be caused by an action of an acid group included in the alkali-soluble resin, such as a carboxy group.

In response to this problem, the present inventors have found that the corrosion of the conductive layer can be suppressed by using a photosensitive composition layer having an NCO value of more than 0.50 mmol/g.

It is presumed that the reason for this is that the post-baking step generates a sufficient amount of isocyanate groups from the blocked isocyanate compound to react with the acid group of the alkali-soluble resin, and as a result, the corrosion of the conductive layer can be suppressed.

In the second transfer film, it is essential that the NCO value of the photosensitive composition layer is more than 0.50 mmol/g, and it differs from the first transfer film described above in that an NCO value of the blocked isocyanate compound included in the photosensitive composition layer is not specified.

The NCO value of the photosensitive composition layer in the second transfer film is more than 0.50 mmol/g, and from the viewpoint that the effects of the present invention are more excellent, is preferably 0.55 mmol/g or more and more preferably 0.60 mmol/g or more.

From the viewpoint that the effects of the present invention are more excellent, the upper limit value of the NCO value of the photosensitive composition layer in the second transfer film is preferably 1.0 mmol/g or less, more preferably less than 0.80 mmol/g, and still more preferably 0.70 mmol/g or less.

The method for measuring the NCO value of the photosensitive composition layer is as described above, and thus the description thereof will be omitted.

Here, examples of a method of setting the NCO value of the photosensitive composition layer within the above-described range include a method of the first blocked isocyanate compound described in the section of the first transfer film as the blocked isocyanate compound included in the photosensitive composition layer. Examples of other methods include a method of adjusting the content of the blocked isocyanate compound in the photosensitive composition layer.

The components which are included or may be included in the photosensitive composition layer of the second transfer film are the same as those in the photosensitive composition layer of the first transfer film, and thus the description thereof will be omitted.

The physical properties such as the thickness, the refractive index, and the color of the photosensitive composition layer in the second transfer film are also the same as those of the photosensitive composition layer in the first transfer film, and thus the description thereof will be omitted.

The temporary support included in the second transfer film is the same as the temporary support included in first transfer film, and thus the description thereof will be omitted.

The second transfer film may have the same refractive index-adjusting layer as that of the first transfer film. In addition, the second transfer film may have the same other layers as those of the first transfer film.

[Method for Producing Transfer Film]

A method for producing the transfer film (the first transfer film and the second transfer film) according to the embodiment of the present invention is not particularly limited, and a known method can be used. In the following description, the term “transfer film” simply means both the first transfer film and the second transfer film.

Above all, a method of applying a photosensitive composition onto a temporary support and performing a drying treatment as necessary to form a photosensitive composition layer (hereinafter, this method is referred to as a “coating method”) is preferable from the viewpoint that the productivity is excellent.

The photosensitive composition used in the coating method preferably includes the above-described components (for example, the polymerizable compound, the alkali-soluble resin, the polymerization initiator, and the blocked isocyanate compound) constituting the photosensitive composition layer, and a solvent.

As the solvent, an organic solvent is preferable. Examples of the organic solvent include methyl ethyl ketone, propylene glycol monomethyl ether, propylene glycol monomethyl ether acetate (another name: 1-methoxy-2-propyl acetate), diethylene glycol ethyl methyl ether, cyclohexanone, methyl isobutyl ketone, ethyl lactate, methyl lactate, caprolactam, n-propanol, and 2-propanol. As the solvent, a mixed solvent of methyl ethyl ketone and propylene glycol monomethyl ether acetate or a mixed solvent of diethylene glycol ethyl methyl ether and propylene glycol monomethyl ether acetate is preferable.

In addition, as the solvent, an organic solvent (high-boiling-point solvent) having a boiling point of 180° C. to 250° C. can also be used as necessary.

The photosensitive composition may include only one kind of solvent, or may include two or more kinds of solvents.

In a case where the photosensitive composition includes the solvent, the total solid content of the photosensitive composition is preferably 5% to 80% by mass, more preferably 5% to 40% by mass, and still more preferably 5% to 30% by mass to the total mass of the photosensitive composition.

In a case where the photosensitive composition includes the solvent, for example, from the viewpoint of coatability, a viscosity of the photosensitive composition at 25° C. is preferably 1 to 50 mPa·s, more preferably 2 to 40 mPa·s, and still more preferably 3 to 30 mPa·s. The viscosity is measured using a viscometer. As the viscometer, for example, a viscometer (product name: VISCOMETER TV-22) manufactured by Toki Sangyo Co., Ltd. can be suitably used. However, the viscometer is not limited to the above-described viscometer.

In a case where the photosensitive composition includes the solvent, from the viewpoint of coatability, a surface tension of the photosensitive composition at 25° C. is preferably 5 to 100 mN/m, more preferably 10 to 80 mN/m, and still more preferably 15 to 40 mN/m. The surface tension is measured using a tensiometer. As the tensiometer, for example, a tensiometer (product name: Automatic Surface Tensiometer CBVP-Z) manufactured by Kyowa Interface Science Co., Ltd. can be suitably used. However, the tensiometer is not limited to the above-described tensiometer.

Examples of the method for applying the photosensitive composition include a printing method, a spray coating method, a roll coating method, a bar coating method, a curtain coating method, a spin coating method, and a die coating method (that is, a slit coating method).

Examples of a drying method include natural drying, heating drying, and drying under reduced pressure. The above-described methods can be adopted alone or in combination of two or more thereof.

In the present disclosure, the “drying” means removing at least a part of the solvent included in the composition.

In addition, in a case where the transfer film has a protective film, the transfer film can be produced by affixing the protective film to the photosensitive composition layer.

A method for affixing the protective film to the photosensitive composition layer is not particularly limited, and examples thereof include known methods.

Examples of a device for affixing the protective film to the photosensitive composition layer include known laminators such as a vacuum laminator and an auto-cut laminator.

It is preferable that the laminator is equipped with any heatable roller such as a rubber roller and can perform pressing and heating.

The transfer film according to the embodiment of the present invention can be applied to various applications. For example, the transfer film according to the embodiment of the present invention can be applied to an electrode protective film, an insulating film, a flattening film, an overcoat film, a hard coat film, a passivation film, a partition wall, a spacer, a microlens, an optical filter, an antireflection film, an etching resist, a plating member, or the like.

More specific examples thereof include a protective film or an insulating film for a touch panel electrode, a protective film or an insulating film for a printed wiring board, a protective film or an insulating film for a TFT substrate, a color filter, an overcoat film for a color filter, an etching resist for a wiring line formation, and a sacrificing layer in a plating process.

[Method for Producing Laminate]

The photosensitive composition layer can be transferred to an object to be transferred by using the above-described transfer film.

Among these, a method for producing a laminate, including an affixing step of bringing the photosensitive composition layer on the temporary support of the transfer film into contact with a substrate having a conductive layer to affix the photosensitive composition layer to the substrate and obtain a photosensitive composition layer-attached substrate having the substrate, the conductive layer, the photosensitive composition layer, and the temporary support in this order; an exposing step of exposing the photosensitive composition layer in a patterned manner; and a developing step of developing the exposed photosensitive composition layer to form a pattern, in which the producing method further includes, between the affixing step and the exposing step or between the exposing step and the developing step, a peeling step of peeling the temporary support from the substrate with a photosensitive composition layer, is preferable.

Hereinafter, the procedure of the steps will be specifically described.

<Affixing Step>

The affixing step is a step of bringing the photosensitive composition layer on the temporary support of the transfer film into contact with a substrate having a conductive layer to affix the photosensitive composition layer to the substrate and obtain a photosensitive composition layer-attached substrate having the substrate, the conductive layer, the photosensitive composition layer, and the temporary support in this order.

An exposed photosensitive composition layer on the temporary support of the transfer film is brought into contact with the substrate having a conductive layer and affixed to the substrate. By this affixing, the photosensitive composition layer and the temporary support are arranged on the substrate having a conductive layer.

In the above-described affixing, the conductive layer and the surface of the photosensitive composition layer are pressure-bonded so that both are in contact with each other. In the above-described aspect, a pattern obtained after exposure and development can be suitably used as an etching resist in a case of etching the conductive layer.

The pressure-bonding method is not particularly limited, and known transfer methods and laminating methods can be used. Among these, it is preferable to superimpose a surface of the photosensitive composition layer on a substrate having a conductive layer, followed by pressurizing and heating with a roll or the like.

A known laminator such as a vacuum laminator and an auto-cut laminator can be used for the affixing.

The substrate having a conductive layer has a conductive layer on the substrate, and any layer may be formed as necessary. That is, the substrate having the conductive layer is a conductive substrate having at least a substrate and a conductive layer arranged on the substrate.

Examples of the substrate include a resin substrate, a glass substrate, and a semiconductor substrate.

Preferred aspects of the substrate are described, for example, in paragraph 0140 of WO2018/155193A, the contents of which are incorporated herein by reference.

As the conductive layer, from the viewpoint of conductivity and a thin wire forming property, at least one layer selected from the group consisting of a metal layer, a conductive metal oxide layer, a graphene layer, a carbon nanotube layer, and a conductive polymer layer is preferable.

In addition, only one conductive layer may be disposed, or two or more conductive layers may be arranged on the substrate. In a case where two or more conductive layers are arranged, it is preferable to have conductive layers made of different materials.

Preferred aspects of the conductive layers are described, for example, in paragraph 0141 of WO2018/155193A, the contents of which are incorporated herein by reference.

As the substrate having a conductive layer, a substrate having at least one of a transparent electrode or a lead wire is preferable. Such a substrate can be suitably used as a substrate for a touch panel.

The transparent electrode can function suitably as a touch panel electrode. The transparent electrode is preferably composed of a metal oxide film such as indium tin oxide (ITO) and indium zinc oxide (IZO), a metal mesh, and a fine metal wire such as a silver nanowire.

Examples of the fine metal wire include thin wire of silver and copper. Among these, silver conductive materials such as silver mesh and silver nanowire are preferable.

As a material of the lead wire, metal is preferable.

Examples of a metal which is the material of the lead wire include gold, silver, copper, molybdenum, aluminum, titanium, chromium, zinc, manganese, and alloy consisting of two or more kinds of these metal elements. As the material of the lead wire, copper, molybdenum, aluminum, or titanium is preferable, copper is particularly preferable.

<Exposing Step>

The exposing step is a step of exposing the photosensitive composition layer in a patterned manner.

Here, the “pattern exposure” refers to exposure in a form of performing the exposure in a patterned manner, that is, a form in which an exposed portion and an non-exposed portion are present.

Detailed arrangement and specific size of the pattern in the pattern exposure are not particularly limited. A pattern formed by the developing step which will be described later preferably includes thin wires having a width of 20 μm or less, and more preferably includes thin wires having a width of 10 μm or less.

As a light source of the pattern exposure, a light source can be appropriately selected, as long as it can emit light at a wavelength region (for example, 365 nm or 405 nm) at which at least the photosensitive composition layer can be cured. Among these, a main wavelength of the exposure light for the exposure in a patterned manner is preferably 365 nm. The main wavelength is a wavelength having the highest intensity.

Examples of the light source include various lasers, a light emitting diode (LED), an ultra-high pressure mercury lamp, a high pressure mercury lamp, and a metal halide lamp.

An exposure amount is preferably 5 to 200 mJ/cm² and more preferably 10 to 200 mJ/cm².

Preferred aspects of the light source, the exposure amount, and the exposing method used for the exposure are described in, for example, paragraphs [0146] and [0147] of WO2018/155193A, the contents of which are incorporated herein by reference.

<Peeling Step>

The peeling step is a step of peeling the temporary support from the substrate with a photosensitive composition layer between the affixing step and the exposing step, or between the exposing step and the developing step which will be described later.

The peeling method is not particularly limited, and the same mechanism as the cover film peeling mechanism described in paragraphs [0161] and [0162] of JP2010-072589A can be used.

<Developing Step>

The developing step is a step of developing the exposed photosensitive composition layer to form a pattern.

Development of the photosensitive composition layer can be performed using a developer.

As the developer, an alkaline aqueous solution is preferable. Examples of an alkaline compound which can be included in the alkaline aqueous solution include sodium hydroxide, potassium hydroxide, sodium carbonate, potassium carbonate, sodium hydrogen carbonate, potassium hydrogen carbonate, tetramethyl ammonium hydroxide, tetraethyl ammonium hydroxide, tetrapropyl ammonium hydroxide, tetrabutylammonium hydroxide, and choline (2-hydroxyethyltrimethyl ammonium hydroxide).

Examples of the developing method include methods such as puddle development, shower development, spin development, and dip development.

Examples of the developer that is suitably used in the present disclosure include the developer described in paragraph [0194] of WO2015/093271A, and examples of the developing method that is suitably used include the developing method described in paragraph [0195] of WO2015/093271A.

The detailed arrangement and the specific size of the pattern to be formed are not particularly limited, but a pattern from which conductive thin wires described later are obtained is preferably formed. A pattern interval is preferably 8 μm or less and more preferably 6 μm or less. The lower limit is not particularly limited, but is 2 μm or more in many cases.

A pattern formed by the procedure (a cured film of the photosensitive composition layer) is preferably achromatic. Specifically, in an L*a*b* color system, the a* value of the pattern is preferably −1.0 to 1.0, and the b* value of the pattern is preferably −1.0 to 1.0.

<Post-Exposing Step and Post-Baking Step>

The above-described method for producing a laminate may have a step of exposing the pattern obtained by the above-described developing step (post-exposing step) and/or a step of heating (post-baking step) the pattern.

In a case where both of the post-exposing step and the post-baking step are included, it is preferable that the post-baking is carried out after the post-exposure.

<Other Steps>

The method for producing a laminate according to the embodiment of the present invention may include any steps (other steps) other than those described above.

Examples thereof include a step of reducing a visible light reflectivity, which is described in paragraph [0172] of WO2019/022089A, and a step of forming a new conductive layer on an insulating film, which is described in paragraph [0172] of WO2019/022089A, but the other steps are not limited to these steps.

The laminate produced by the method for producing a laminate according to the embodiment of the present invention can be applied to various devices. Examples of the device provided with the above-described laminate include a display device, a printed wiring board, a semiconductor package, and an input device, and a touch panel is preferable, and a capacitance type touch panel is more preferable. In addition, the above-described input device can be applied to a display device such as an organic electroluminescent display device and a liquid crystal display device.

In a case where the laminate is applied to a touch panel, it is preferable that the pattern formed from the photosensitive composition layer is used as a protective film for a touch panel electrode. That is, it is preferable that the photosensitive composition layer included in the transfer film is used for formation of a touch panel electrode protective film. The touch panel electrode includes not only a sensor electrode of the touch sensor but also a lead wire.

[Blocked Isocyanate Compound Represented by Formula QA]

The blocked isocyanate compound according to the embodiment of the present invention is a blocked isocyanate compound represented by Formula QA, and is a blocked isocyanate compound having a novel structure.

B^{1a}-A^{1a}-L^{1a}-A^{2a}-B^{2a} Formula QA

Definitions and suitable aspects of B^{1a} A^{1a}, L^{1a}, A^{2a}, and B^{2a} in Formula QA are as described above, and thus the description thereof will be omitted.

The compound represented by Formula QA is obtained, for example, by reacting an isocyanate group of a compound having an isocyanate group (for example, a compound in which B^{1a} and B^{2a} in Formula QA described above are isocyanate groups) with the above-described blocking agent.

Reaction conditions between the compound having an isocyanate group and the blocking agent are not particularly limited, and the same reaction conditions as known blocked isocyanate compounds can be adopted.

The blocked isocyanate compound represented by Formula QA is preferably a blocked isocyanate compound represented by Formula Q-1.

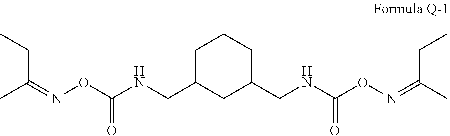 [SMILES: CC/C(C)=N\OC(=O)NCC1CCCC(CNC(=O)O/N=C(/C)CC)C1]

The blocked isocyanate compound represented by Formula Q-1 may be an isomer mixture of a cis form and a trans form (hereinafter, also referred to as a “cis-trans isomer mixture”).

In a case where the blocked isocyanate compound represented by Formula Q-1 is the cis-trans isomer mixture, a mass ratio of the cis form to the trans form is preferably cis form/trans form=10/90 to 90/10, and more preferably cis form/trans form=40/60 to 60/40.

Applications of the compound represented by Formula QA are not particularly limited, but are particularly suitable as a component for forming the photosensitive composition layer in the above-described transfer film.

[Specific Example of Touch Panel]

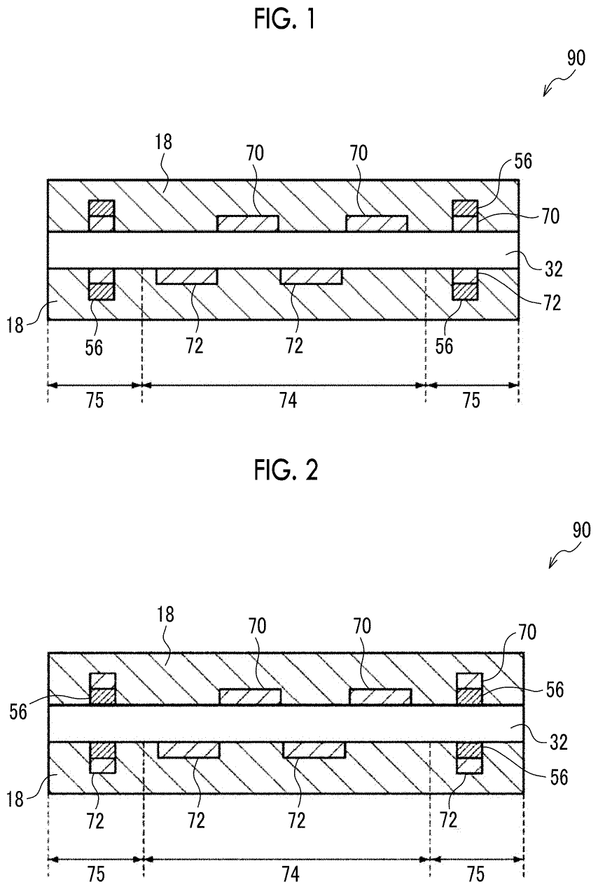

FIG. 1 is a schematic cross-sectional view showing a specific example of a touch panel 90 that is a first specific example to which the transfer film according to the embodiment of the present invention can be applied.

As shown in FIG. 1, the touch panel 90 has an image display region 74 and an image non-display region 75 (that is, a frame portion).

In addition, the touch panel 90 includes the electrode for a touch panel on both surfaces of a substrate 32. Specifically, the touch panel 90 includes a first metal conductive material 70 on one surface of the substrate 32 and includes a second metal conductive material 72 on the other surface thereof.

In the touch panel 90, a lead wire 56 is connected to the first metal conductive material 70 and the second metal conductive material 72, respectively. The lead wire 56 is, for example, a copper wire or a silver wire.

In the touch panel 90, a metal conductive material protective film 18 is formed on one surface of the substrate 32 so as to cover the first metal conductive material 70 and the lead wire 56, and the metal conductive material protective film 18 is formed on the other surface of the substrate 32 so as to cover the second metal conductive material 72 and the lead wire 56.

A refractive index-adjusting layer may be formed on one surface of the substrate 32.

In addition, FIG. 2 is a schematic cross-sectional view showing a specific example of a touch panel 90 that is a second specific example to which the transfer film according to the embodiment of the present invention can be applied.

As shown in FIG. 2, the touch panel 90 has an image display region 74 and an image non-display region 75 (that is, frame portion).

In addition, the touch panel 90 includes the electrode for a touch panel on both surfaces of a substrate 32. Specifically, the touch panel 90 includes a first metal conductive material 70 on one surface of the substrate 32 and includes a second metal conductive material 72 on the other surface thereof.

In the touch panel 90, a lead wire 56 is connected to the first metal conductive material 70 and the second metal conductive material 72, respectively. The lead wire 56 is, for example, a copper wire or a silver wire. In addition, the lead wire 56 is formed inside surrounded by the metal conductive material protective film 18, and the first metal conductive material 70 or the second metal conductive material 72.

In the touch panel 90, a metal conductive material protective film 18 is formed on one surface of the substrate 32 so as to cover the first metal conductive material 70 and the lead wire 56, and the metal conductive material protective film 18 is formed on the other surface of the substrate 32 so as to cover the second metal conductive material 72 and the lead wire 56.

A refractive index-adjusting layer may be formed on one surface of the substrate 32.

It is preferable that the metal conductive material protective film 18 is the photosensitive composition layer or the cured film of the photosensitive composition layer in the present invention.

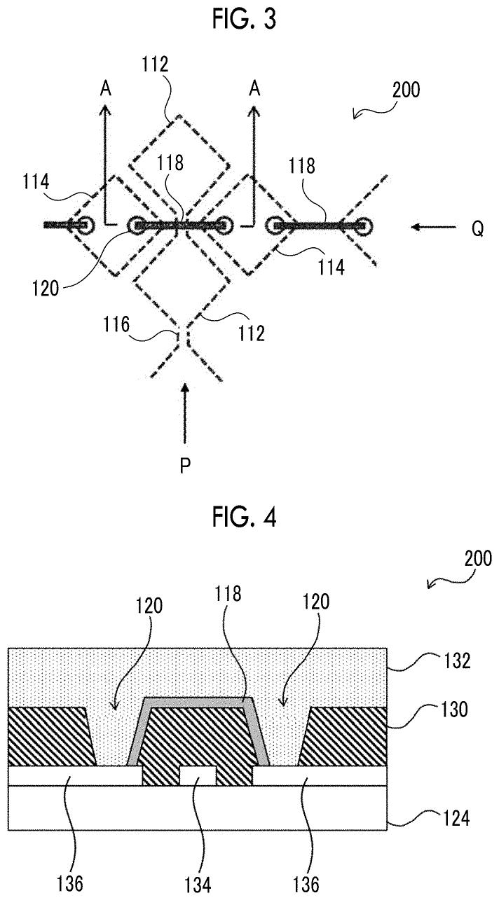

Still another embodiment of the touch panel will be described with reference to FIGS. 3 and 4.

FIG. 3 is a schematic plan view showing still another specific example of the touch panel, and FIG. 4 is a cross-sectional view taken along a line A-A of FIG. 3.

FIGS. 3 and 4 show a transparent laminate 200 having a transparent electrode pattern (including a first island-shaped electrode portion, a first wiring part 116, a second island-shaped electrode portion, and a bridge wire 118), a protective layer 130, and an overcoat layer 132 in this order on a transparent film substrate 124.

It is preferable that at least one of the protective layer 130 or the overcoat layer 132 is the photosensitive composition layer or the cured film of the photosensitive composition layer in the present invention.

In addition, as shown in FIGS. 3 and 4, on the protective layer 130 disposed on the second island-shaped electrode portion 114 in the transparent electrode pattern on the transparent film substrate 124, a through hole 120 for connecting the second island-shaped electrode portion 114 and the bridge wire (second wiring part) 118 for bridging between two second island-shaped electrode portions 114 adjacent to each other and electrically connecting the second island-shaped electrode portions 114 to each other is formed.

The transparent laminate 200 has, on the transparent substrate 124, a first electrode pattern 134 and a second electrode pattern 136, which respectively extends in a direction of an arrow P or a direction of an arrow Q.

FIGS. 3 and 4 show only a part of the touch panel, but on the transparent substrate, the first electrode patterns 134 are arranged in one direction (first direction) over a wide range of the transparent substrate, and furthermore, the second electrode patterns 136 are arranged in a direction (second direction) different from the first direction over a wide range of the transparent substrate.

In FIG. 3, the first electrode pattern 134 is disposed on the transparent substrate 124 such that a plurality of rectangular electrode parts (first island-shaped electrode portions) 112 are arranged in an island shape at equal intervals along the direction of the arrow P, and the first island-shaped electrode portions 112 adjacent to each other are continuously connected by the first wiring part 116. As a result, an elongated electrode is formed in one direction on the surface of the transparent substrate.

The first wiring part is preferably formed of the same material as the first island-shaped electrode portion.

In addition, in FIG. 3, the second electrode pattern 136 is disposed on the transparent substrate 124 such that rectangular electrode parts (second island-shaped electrode portions) 114 which are substantially the same as the first island-shaped electrode portion are arranged in an island shape at equal intervals along the direction of the arrow Q, which is substantially perpendicular to the direction of the arrow P, and the second island-shaped electrode portions 114 adjacent to each other are continuously connected by the second wiring part (bridge wire) 118.

As a result, an elongated electrode is formed in one direction different from the first electrode pattern on the surface of the transparent substrate.

As shown in FIGS. 3 and 4, the first electrode pattern 134 and the second electrode pattern 136 form a bridge structure in which one of intersecting electrodes jumps over the other at an intersecting portion so as to prevent the first electrode pattern 134 and the second electrode pattern 136 from conducting each other.

In the touch panel shown in FIG. 4, the protective layer 130 is disposed so as to cover the first electrode pattern 134 and the second electrode pattern 136.

### EXAMPLES

Hereinafter, the present invention will be described in detail with reference to Examples. The material, the amount used, the ratio, the process contents, the process procedure, and the like shown in the following examples can be appropriately changed, within a range not departing from a gist of the present disclosure. Accordingly, the scope of the present invention is not limited to the following specific examples. “part” and “%” are based on mass unless otherwise specified.

In the following examples, a weight-average molecular weight of a resin is a weight-average molecular weight obtained by performing polystyrene conversion of a value measured by gel permeation chromatography (GPC). Further, a theoretical acid value was used as the acid value.

<Synthesis of Alkali-Soluble Resin P-1>

82.4 g of propylene glycol monomethyl ether was charged into a flask and heated to 90° C. under a nitrogen stream. To this liquid, a solution in which 38.4 g of styrene, 30.1 g of dicyclopentanyl methacrylate, and 34.0 g of methacrylic acid had been dissolved in 20 g of propylene glycol monomethyl ether and a solution in which 5.4 g of a polymerization initiator V-601 (manufactured by FUJIFILM Wako Pure Chemical Corporation) had been dissolved in 43.6 g of propylene glycol monomethyl ether acetate were simultaneously added dropwise over 3 hours. After the dropwise addition, 0.75 g of V-601 was added three times every hour. Thereafter, the reaction was continued for another 3 hours. Thereafter, the reaction liquid was diluted with 58.4 g of propylene glycol monomethyl ether acetate and 11.7 g of propylene glycol monomethyl ether. The reaction liquid was heated to 100° C. under an air stream, and 0.53 g of tetraethylammonium bromide and 0.26 g of p-methoxyphenol were added thereto. 25.5 g of glycidyl methacrylate (Blemmer GH manufactured by NOF Corporation.) was added dropwise thereto over 20 minutes. The mixture was reacted at 100° C. for 7 hours to obtain a solution of an alkali-soluble resin P-1. The concentration of solid contents of the obtained solution was 36.5%. In the alkali-soluble resin P-1, the weight-average molecular weight in terms of standard polystyrene in GPC was 17000, the dispersity was 2.4, and the acid value was 94.5 mgKOH/g. The amount of residual monomer measured by gas chromatography was less than 0.1% by mass with respect to the solid content of the polymer in any of the monomers.

<Synthesis of Alkali-soluble Resins P-2 to P-19>

Alkali-soluble resins P-2 to P-19 were synthesized in the same manner as the synthesis of the alkali-soluble resin P-1, except that the types of monomers for obtaining each structural unit included in the alkali-soluble resin and the content of each structural unit were changed as shown in Table 1. All of the alkali-soluble resins were synthesized as a polymer solution, and the amount of the diluent (propylene glycol monomethyl ether acetate (PGMEA)) was adjusted so that the concentration (concentration of solid contents) of the alkali-soluble resin in the polymer solution was 36.3% by mass.

In Table 1, structural units other than structural units having a radically polymerizable group are indicated by abbreviations of monomers for forming each structural unit.

The structural unit having a radically polymerizable group is indicated in the form of a monomer and an addition structure of a monomer. For example, MAA-GMA means a structural unit in which glycidyl methacrylate is added to a structural unit derived from methacrylic acid.

In Table 1, meanings of the abbreviations are as follows.

St: styrene (manufactured by Wako Pure Chemical Industries, Ltd.)

VN: vinyl naphthalene (manufactured by Wako Pure Chemical Industries, Ltd.)

AMS: α-methylstyrene (manufactured by Tokyo Chemical Industry Co., Ltd.)

DCPMA: dicyclopentanyl methacrylate (Tg: 175° C., FANCRYL FA-513M, manufactured by Hitachi Chemical Co., Ltd.)

IBXMA: isobornyl methacrylate (Tg: 173° C., LIGHT ESTER IB-X, manufactured by KYOEISHA CHEMICAL Co., LTD.)

ADMA: 1-adamantyl methacrylate (Tg: 250° C., Adamantate AM (manufactured by Idemitsu Kosan Co., Ltd.))

CHMA: cyclohexyl methacrylate (Tg=66° C., CHMA, MITSUBISHI manufactured by GAS CHEMICAL COMPANY, INC.)

MAA-GMA: structural unit in which glycidyl methacrylate is added to a structural unit derived from methacrylic acid

MAA-M100: structural unit in which CYM-M100 (manufactured by Daicel Corporation; 3,4-epoxycyclohexylmethylmethacrylate) is added to a structural unit derived from methacrylic acid

MAA: methacrylic acid (manufactured by Wako Pure Chemical Industries, Ltd.)

AA: acrylic acid (manufactured by Wako Pure Chemical Industries, Ltd.)

MMA: methyl methacrylate (manufactured by Wako Pure Chemical Industries, Ltd.)

nBMA: n-butyl methacrylate (manufactured by Wako Pure Chemical Industries, Ltd.)

HEMA: hydroxyethyl methacrylate (manufactured by Wako Pure Chemical Industries, Ltd.)

4HBA: 4-hydroxybutyl acrylate (manufactured by Wako Pure Chemical Industries, Ltd.)

TABLE 1

| Al [symbol] li- [symbol] resin | | P-1 | P-2 | P-3 | P-4 | P-5 | P-6 | P-7 | P-8 | P-9 | P-10 |
|---|---|---|---|---|---|---|---|---|---|---|---|
| Str [symbol] ural unit A1 (% by mass) | S [symbol] | 30 | — | — | 45 | 50 | 55 | 48.9 | 48 | 49.2 | 49.2 |
| | VN | — | 35 | — | — | — | — | — | — | — | — |
| | AMS | — | — | 40 | — | — | — | — | — | — | — |
| St [symbol] al unit B1 (% by mass) | MAA-GMA | 32 | — | 40 | 20 | 32 | 29 | 32 | 32 | 32 | 32 |
| | MAA-M [symbol] 00 | — | 32 | — | — | — | — | — | — | — | — |
| St [symbol] al unit C1 (% by mass) | MAA | 14.5 | — | 14.5 | 16 | 16 | 14 | 16 | 16 | 1 [symbol] .5 | 17.5 |
| | AA | — | 14.5 | — | — | — | — | — | — | — | — |
| Structural unit D1 (% by mass) | DCPMA | 23.5 | — | — | — | — | — | — | 2 | — | — |
| | IBXMA | — | 18.5 | — | — | — | — | — | — | — | — |
| | ADMA | — | — | 5.5 | — | — | — | — | — | — | — |
| | CHMA | — | — | — | 19 | — | — | — | — | — | — |
| | MMA | — | — | — | — | 2 | — | 1.3 | — | 1.3 | 1.3 |
| | nBMA | — | — | — | — | — | 2 | — | — | — | — |
| Other s [symbol] al units (% by [symbol] ss) | HEMA | — | — | — | — | — | — | 1.8 | — | — | — |
| | 4HBA | — | — | — | — | — | — | — | 2 | — | — |

| | | | | | | | | | | |
|---|---|---|---|---|---|---|---|---|---|---|
| Total (% by mass) | 100 | 100 | 100 | 100 | 100 | 100 | 100 | 100 | 100 | 100 |
| Weight-average molecular weight Mw | 17000 | 20000 | 25000 | 12000 | 8000 | 30000 | 18000 | 20000 | 18000 | 11000 |
| Dispersity | 2.4 | 2.4 | 2.1 | 2.6 | 2.1 | 2.4 | 2.4 | 2.4 | 2.4 | 2.3 |

| Al*li- [symbol] resin | | P-11 | P-12 | P-13 | P-14 | P-15 | P-16 | P-17 | P-18 | P-19 |
|---|---|---|---|---|---|---|---|---|---|---|
| Str [symbol] ural unit A1 (% by mass) | S [symbol] | 47.7 | 47.7 | 50.2 | 50.5 | 47.4 | 48.9 | 44.7 | 50.2 | — |
| | VN | — | — | — | — | — | — | — | — | — |
| | AMS | — | — | — | — | — | — | — | — | — |
| St [symbol] al unit B1 (% by mass) | MAA-GMA | 32 | 32 | 32 | 32 | 32 | 32 | 32 | 26.5 | 29 |
| | MAA-M [symbol] 00 | — | — | — | — | — | — | — | — | — |
| St [symbol] al unit C1 (% by mass) | MAA | 19 | 19 | 17.5 | 1 [symbol] | 17.5 | 17.5 | 23 | 23 | 15 |
| | AA | — | — | — | — | — | — | — | — | — |
| Structural unit D1 (% by mass) | DCPMA | — | — | — | — | — | — | — | — | — |
| | IBXMA | — | — | — | — | — | — | — | — | — |
| | ADMA | — | — | — | — | — | — | — | — | — |
| | CHMA | — | — | — | 0.5 | — | — | — | — | 55 |
| | MMA | 1.3 | 1.3 | 0.3 | — | 1.3 | 1.3 | 0.3 | 0.3 | 1 |
| | nBMA | — | — | — | — | — | — | — | — | — |
| Other s [symbol] al units (% by [symbol] ss) | HEMA | — | — | — | — | 1.8 | 0.3 | — | — | — |
| | 4HBA | — | — | — | — | — | — | — | — | — |

| | | | | | | | | | |
|---|---|---|---|---|---|---|---|---|---|
| Total (% by mass) | 100 | 100 | 100 | 100 | 100 | 100 | 100 | 100 | 100 |
| Weight-average molecular weight Mw | 17000 | 12000 | 16000 | 13000 | 24000 | 30000 | 16000 | 15000 | 27000 |
| Dispersity | 2.4 | 2.4 | 2.4 | 2.4 | 2.4 | 2.2 | 2.4 | 2.4 | 2.2 |

[symbol]

indicates data missing or illegible when filed

<Synthesis of Blocked Isocyanate Compound Q-1>

Under a nitrogen stream, 453 g of butanone oxime (manufactured by Idemitsu Kosan Co., Ltd.) was dissolved in 700 g of methyl ethyl ketone. 500 g of 1,3-bis(isocyanatomethyl)cyclohexane (cis, trans isomer mixture, manufactured by Mitsui Chemicals Inc., TAKENATE 600) was added dropwise thereto over 1 hour under ice-cooling, and the reaction was performed for another 1 hour after the dropwise addition. Thereafter, the temperature was raised to 40° C. and the reaction was performed for 1 hour. It was confirmed by ¹H-nuclear magnetic resonance (NMR) and high performance liquid chromatography (HPLC) that the reaction was completed to obtain a methyl ethyl ketone solution of a blocked isocyanate compound Q-1 (see the following formula).

<Synthesis of Blocked Isocyanate Compound Q-1-A>

A methyl ethyl ketone solution of a blocked isocyanate compound Q-1-A was obtained with reference to the synthesis of the blocked isocyanate compound Q-1. The amount of butanone oxime in the solution was 0.3 parts by mass with respect to 100 parts by mass of Q-1-A.

<Synthesis of Blocked Isocyanate Compound Q-1-B>

A methyl ethyl ketone solution of a blocked isocyanate compound Q-1-B was obtained with reference to the synthesis of the blocked isocyanate compound Q-1-A. The amount of butanone oxime in the solution was 1.2 parts by mass with respect to 100 parts by mass of Q-1-B.

<Synthesis of Blocked Isocyanate Compounds Q-2 to Q-8>

A methyl ethyl ketone solution of blocked isocyanate compounds Q-2 to Q-8 (see the following formulae) was obtained with reference to the synthesis method of the blocked isocyanate compound Q-1. The blocked isocyanate compound Q-6 is a 1:1 (mass ratio) mixture of isomers.

| Blocked isocyanate | | NCO value |
|---|---|---|
| compound | Structure | [mmol/g] |
| Q-1 | 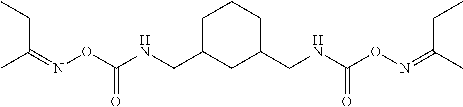 [SMILES: CC/C(C)=N\OC(=O)NCC1CCCC(CNC(=O)O/N=C(/C)CC)C1] | 5.4 |
| Q-1-A | 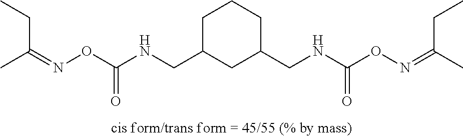 [SMILES: CC/C(C)=N\OC(=O)NCC1CCCC(CNC(=O)O/N=C(/C)CC)C1] | 5.4 |
| Q-1-B | 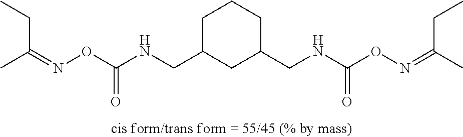 [SMILES: CC/C(C)=N\OC(=O)NCC1CCCC(CNC(=O)O/N=C(/C)CC)C1] | 5.4 |
| Q-2 | 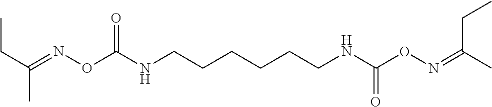 [SMILES: CC/C(C)=N\OC(=O)NCCCCCCNC(=O)O/N=C(\C)CC] | 5.8 |
| Q-3 | 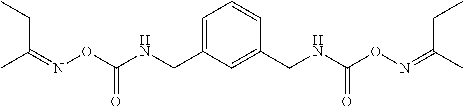 [SMILES: CC/C(C)=N\OC(=O)NCc1cccc(CNC(=O)O/N=C(/C)CC)c1] | 5.5 |
| Q-4 | 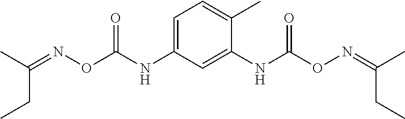 [SMILES: CC/C(C)=N\OC(=O)Nc1ccc(C)c(NC(=O)O/N=C(/C)CC)c1] | 5.7 |
| Q-5 | 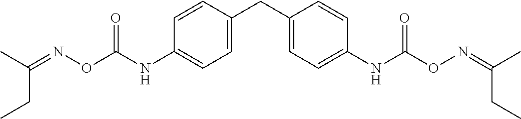 [SMILES: CC/C(C)=N\OC(=O)Nc1ccc(Cc2ccc(NC(=O)O/N=C(/C)CC)cc2)cc1] | 4.7 |
| Q-6 | 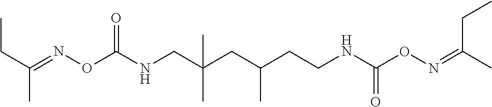 [SMILES: CC/C(C)=N\OC(=O)NCCC(C)CC(C)(C)CNC(=O)O/N=C(\C)CC] | 5.2 |
| | 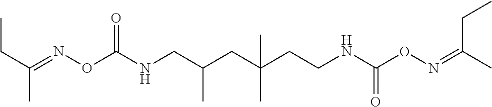 [SMILES: CC/C(C)=N\OC(=O)NCCC(C)(C)CC(C)CNC(=O)O/N=C(\C)CC] | |
| Q-7 | 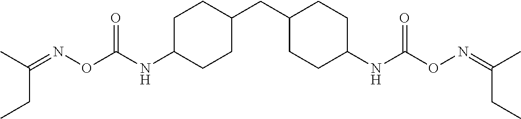 [SMILES: CC/C(C)=N\OC(=O)NC1CCC(CC2CCC(NC(=O)O/N=C(/C)CC)CC2)CC1] | 4.6 |
| Q-8 | 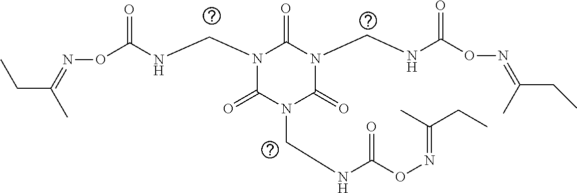 [SMILES: CC/C(C)=N/OC(=O)NCn1c(=O)n(CNC(=O)O/N=C(\C)CC)c(=O)n(CNC(=O)O/N=C(\C)CC)c1=O] | 3.9 |

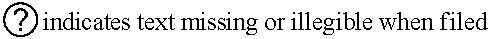

NCO values of the blocked isocyanate compounds Q-1 to Q-8 were measured according to the method described above.

<Preparation of Photosensitive Composition>

Photosensitive compositions A-1 to A-38 and A-1 having compositions shown in Table 2 were prepared. In Table 2, a numerical value of each component represents the content (solid content mass) of each component. Methyl ethyl ketone and 1-methoxy-2-propyl acetate were appropriately added such that the content of the methyl ethyl ketone in the solvent was 60% by mass and that the concentration of solid contents in A- to A-31 was 25% by mass or the concentration of solid contents in A-32 to A-38 was 20% by mass, thereby preparing a coating liquid of the photosensitive composition.

TABLE 2
Table 2 (1)

| | | | A-1 | A-2 | A-3 | A-4 | A-5 | A-6 | A-7 |
|---|---|---|---|---|---|---|---|---|---|
| Polymerizable | Tricyclodecane dimethanol diacrylate (A-DCP, manufactured | | 17.90 | 17.90 | 17.90 | 17.90 | 17.90 | 17.90 | 17.90 |
| compound | by Shin-Nakamura Chemical Co., Ltd.) | | | | | | | | |
| | Monomer having carboxy group ARONIX TO-2349 | | 2.98 | 2.98 | 2.98 | 2.98 | 2.98 | 2.98 | 2.98 |
| | (manufactured by Toagosei Co., Ltd.) | | | | | | | | |
| | Urethane acrylate 8UX-015A (manufactured by Taisei Fine | | — | — | 10.72 | — | — | — | — |
| | Chemical Co., Ltd.) | | | | | | | | |
| | A-NOD-N (manufactured by Shin-Nakamura Chemical Co., Ltd.) | | 2.73 | 2.73 | — | 2.73 | 2.73 | 2.73 | 2.73 |
| | A-DPH (manufactured by Shin-Nakamura Chemical Co., Ltd.) | | 7.99 | 7.99 | — | 7.99 | 7.99 | 7.99 | 7.99 |
| Alkali-soluble | P-1 | | — | — | — | — | — | — | — |
| resin | P-2 | | — | — | — | — | — | — | — |
| | P-3 | | — | — | — | — | — | — | — |
| | P-4 | | — | — | — | — | — | — | — |
| | P-5 | | — | — | — | — | — | — | — |
| | P-6 | | — | — | — | — | — | — | — |
| | P-7 | | — | — | — | — | — | — | — |
| | P-8 | | — | — | — | — | — | — | — |
| | P-9 | | — | — | — | — | — | — | — |
| | P-10 | | — | — | — | — | — | — | — |
| | P-11 | | 52.67 | 52.67 | 52.67 | 52.67 | 52.67 | 52.67 | 52.67 |
| | P-12 | | — | — | — | — | — | — | — |
| | P-13 | | — | — | — | — | — | — | — |
| | P-14 | | — | — | — | — | — | — | — |
| | P-15 | | — | — | — | — | — | — | — |
| | P-16 | | — | — | — | — | — | — | — |
| | P-17 | | — | — | — | — | — | — | — |
| | P-18 | | — | — | — | — | — | — | — |
| | P-19 | | — | — | — | — | — | — | — |
| Photopoly- | 1-[9-ethyl-6-(2-methylbenzoyl)-9H-carbazol-3-yl]ethanone-1- | | 0.36 | — | 0.36 | 0.36 | 0.36 | 0.36 | 0.36 |
| merization | (O-acetyloxime) (OXE-02, manufactured by BASF SE) | | | | | | | | |
| initiator | OXE-03, manufactured by BASF SE | | — | 0.36 | — | — | — | — | — |
| | 2-methyl-1-(4-methylthiophenyl)-2-morpholinopropan-1-one | | 0.73 | — | 0.73 | 0.73 | 0.73 | 0.73 | 0.73 |
| | (Irgacure907, manufactured by BASF SE) | | | | | | | | |
| | 1-(biphenyl-4-yl)-2-methyl-2-morpholinopropan-1-one (APi- | | — | 0.73 | — | — | — | — | — |
| | 307, manufactured by Shenzhen UV-ChemTech Co., Ltd.) | | | | | | | | |
| Blocked | Q-1 | 5.40 | 12.50 | | | | | | |
| isocyanate | Q-1-A | 5.40 | | | | | | | |
| compound | Q-1-B | 5.40 | | | | | | | |
| | Q-2 | 5.80 | | 12.50 | | | | | |
| | Q-3 | 5.50 | | | 12.50 | | | | |
| | Q-4 | 5.70 | | | | 12.50 | | | |
| | Q-5 | 4.70 | | | | | 12.50 | | |
| | Q-6 | 5.20 | | | | | | 12.50 | |
| | Q-7 | 4.60 | | | | | | | 12.50 |
| | Q-8 | 3.90 | | | | | | | |

| | | | | | | | | |
|---|---|---|---|---|---|---|---|---|
| Additive | N-phenylglycine (manufactured by Tokyo Chemical Industry | 0.10 | 0.10 | 0.10 | 0.10 | 0.10 | 0.10 | 0.10 |
| | Co., Ltd.) | | | | | | | |
| | 1,2,4-triazole (manufactured by Otsuka Chemical Co., Ltd.) | — | 0.13 | — | — | — | — | — |
| | Benzoimidazole (manufactured by Tokyo Chemical Industry | 0.13 | — | — | 0.13 | 0.13 | 0.13 | 0.13 |
| | Co., Ltd.) | | | | | | | |
| | 5-amino-1H-tetrazole (manufactured by Tokyo Chemical | — | — | 0.13 | — | — | — | — |
| | Industry Co., Ltd.) | | | | | | | |
| | Isonicotinamide (manufactured by Tokyo Chemical Industry | 0.52 | 0.52 | 0.52 | 0.52 | 0.52 | 0.52 | 0.52 |
| | Co., Ltd.) | | | | | | | |
| | SMA EF-40 (manufactured by TOMOEGAWA CO., LTD.) | 1.20 | 1.20 | 1.20 | 1.20 | 1.20 | 1.20 | 1.20 |
| | MEGAFACE F551A (manufactured by DIC Corporation) | 0.19 | 0.19 | 0.19 | 0.19 | 0.19 | 0.19 | 0.19 |
| | MEGAFACE R-41 (manufactured by DIC Corporation) | | | | | | | |
| | DOWSIL 8032 ADDITIVE (manufactured by Dow Corning | | | | | | | |
| | Toray Co., Ltd.) | | | | | | | |
| | Ftergent 710FL (manufactured by Neos Corporation) | | | | | | | |

| | | | | | | | |
|---|---|---|---|---|---|---|---|
| Concentration of solid contents of coating liquid | 25% | 25% | 25% | 25% | 25% | 25% | 25% |

TABLE 3
Table 2 (2)

| | | | A-8 | A-9 | A-10 | A-11 | A-12 | A-13 | A-14 |
|---|---|---|---|---|---|---|---|---|---|
| Polymerizable | Tricyclodecane dimethanol diacrylate (A-DCP, manufactured | | 18.26 | 18.26 | 18.26 | 18.26 | 18.26 | 18.26 | 18.26 |
| compound | by Shin-Nakamura Chemical Co., Ltd.) | | | | | | | | |
| | Monomer having carboxy group ARONIX TO-2349 | | 3.04 | 3.04 | 3.04 | 3.04 | 3.04 | 3.04 | 3.04 |
| | (manufactured by Toagosei Co., Ltd.) | | | | | | | | |
| | Urethane acrylate 8UX-015A (manufactured by Taisei Fine | | — | — | — | — | — | — | — |
| | Chemical Co., Ltd.) | | | | | | | | |
| | A-NOD-N (manufactured by Shin-Nakamura Chemical Co., Ltd.) | | 2.79 | 2.79 | 2.79 | 2.79 | 2.79 | 2.79 | 2.79 |
| | A-DPH (manufactured by Shin-Nakamura Chemical Co., Ltd.) | | 8.15 | 8.15 | 8.15 | 8.15 | 8.15 | 8.15 | 8.15 |
| Alkali-soluble | P-1 | | 49.03 | 49.03 | 49.03 | 49.03 | 49.03 | 49.03 | 49.03 |
| resin | P-2 | | — | — | — | — | — | — | — |
| | P-3 | | — | — | — | — | — | — | — |
| | P-4 | | — | — | — | — | — | — | — |
| | P-5 | | — | — | — | — | — | — | — |
| | P-6 | | — | — | — | — | — | — | — |
| | P-7 | | — | — | — | — | — | — | — |
| | P-8 | | — | — | — | — | — | — | — |
| | P-9 | | — | — | — | — | — | — | — |
| | P-10 | | — | — | — | — | — | — | — |
| | P-11 | | — | — | — | — | — | — | — |
| | P-12 | | — | — | — | — | — | — | — |
| | P-13 | | — | — | — | — | — | — | — |
| | P-14 | | — | — | — | — | — | — | — |
| | P-15 | | — | — | — | — | — | — | — |
| | P-16 | | — | — | — | — | — | — | — |
| | P-17 | | — | — | — | — | — | — | — |
| | P-18 | | — | — | — | — | — | — | — |
| | P-19 | | — | — | — | — | — | — | — |
| Photopoly- | 1-[9-ethyl-6-(2-methylbenzoyl)-9H-carbazol-3-yl]ethanone-1- | | 0.37 | 0.37 | 0.37 | 0.37 | 0.37 | 0.37 | 0.37 |
| merization | (O-acetyloxime) (OXE-02, manufactured by BASF SE) | | | | | | | | |
| initiator | OXE-03, manufactured by BASF SE | | — | — | — | — | — | — | — |
| | 2-methyl-1-(4-methylthiophenyl)-2-morpholinopropan-1-one | | 0.74 | 0.74 | 0.74 | 0.74 | 0.74 | 0.74 | 0.74 |
| | (Irgacure907, manufactured by BASF SE) | | | | | | | | |
| | 1-(biphenyl-4-yl)-2-methyl-2-morpholinopropan-1-one (APi- | | — | — | — | — | — | — | — |
| | 307, manufactured by Shenzhen UV-ChemTech Co., Ltd.) | | | | | | | | |
| Blocked | Q-1 | 5.40 | 2.02 | 3.09 | 5.16 | | | | |
| isocyanate | Q-1-A | 5.40 | | | | | | | |
| compound | Q-1-B | 5.40 | | | | | | | |
| | Q-2 | 5.80 | | | | | | | |
| | Q-3 | 5.50 | | | | 6.37 | | | 5.16 |
| | Q-4 | 5.70 | | | | | 4.42 | | |
| | Q-5 | 4.70 | | | | | | 2.58 | |
| | Q-6 | 5.20 | | | | | | | |
| | Q-7 | 4.60 | | | | | | | |
| | Q-8 | 3.90 | 13.46 | 12.38 | 10.32 | 9.10 | 11.05 | 12.89 | 10.32 |

| | | | | | | | | |
|---|---|---|---|---|---|---|---|---|
| Additive | N-phenylglycine (manufactured by Tokyo Chemical Industry | 0.10 | 0.10 | 0.10 | 0.10 | 0.10 | 0.10 | 0.10 |
| | Co., Ltd.) | | | | | | | |
| | 1,2,4-triazole (manufactured by Otsuka Chemical Co., Ltd.) | — | — | — | — | — | — | — |
| | Benzoimidazole (manufactured by Tokyo Chemical Industry | 0.13 | 0.13 | 0.13 | 0.13 | 0.13 | 0.13 | 0.13 |
| | Co., Ltd.) | | | | | | | |
| | 5-amino-1H-tetrazole (manufactured by Tokyo Chemical | — | — | — | — | — | — | — |
| | Industry Co., Ltd.) | | | | | | | |
| | Isonicotinamide (manufactured by Tokyo Chemical Industry | 0.52 | 0.52 | 0.52 | 0.52 | 0.52 | 0.52 | 0.52 |
| | Co., Ltd.) | | | | | | | |
| | SMA EF-40 (manufactured by TOMOEGAWA CO., LTD.) | 1.20 | 1.20 | 1.20 | 1.20 | 1.20 | 1.20 | 1.20 |
| | MEGAFACE F551A (manufactured by DIC Corporation) | 0.19 | 0.19 | 0.19 | 0.19 | 0.19 | 0.19 | 0.19 |
| | MEGAFACE R-41 (manufactured by DIC Corporation) | | | | | | | |
| | DOWSIL 8032 ADDITIVE (manufactured by Dow Corning | | | | | | | |
| | Toray Co., Ltd.) | | | | | | | |
| | Ftergent 710FL (manufactured by Neos Corporation) | | | | | | | |

| | | | | | | | |
|---|---|---|---|---|---|---|---|
| Concentration of solid contents of coating liquid | 25% | 25% | 25% | 25% | 25% | 25% | 25% |

TABLE 4
Table 2 (3)

| | | | A-15 | A-16 | A-17 | A-18 | A-19 | A-20 | A-21 |
|---|---|---|---|---|---|---|---|---|---|
| Polymerizable | Tricyclodecane dimethanol diacrylate (A-DCP, manufactured | | 18.26 | 18.26 | 18.26 | 18.26 | 18.26 | 18.26 | 18.26 |
| compound | by Shin-Nakamura Chemical Co., Ltd.) | | | | | | | | |
| | Monomer having carboxy group ARONIX TO-2349 | | 3.04 | 3.04 | 3.04 | 3.04 | 3.04 | 3.04 | 3.04 |
| | (manufactured by Toagosei Co., Ltd.) | | | | | | | | |
| | Urethane acrylate 8UX-015A (manufactured by Taisei Fine | | — | — | — | — | — | — | — |
| | Chemical Co., Ltd.) | | | | | | | | |
| | A-NOD-N (manufactured by Shin-Nakamura Chemical Co., Ltd.) | | 2.79 | 2.79 | 2.79 | 2.79 | 2.79 | 2.79 | 2.79 |
| | A-DPH (manufactured by Shin-Nakamura Chemical Co., Ltd.) | | 8.15 | 8.15 | 8.15 | 8.15 | 8.15 | 8.15 | 8.15 |
| Alkali-soluble | P-1 | | — | — | — | — | — | — | — |
| resin | P-2 | | 49.03 | — | — | — | — | — | — |
| | P-3 | | — | 49.03 | — | — | — | — | — |
| | P-4 | | — | — | 49.03 | — | — | — | — |
| | P-5 | | — | — | — | 49.03 | — | — | — |
| | P-6 | | — | — | — | — | 49.03 | — | — |
| | P-7 | | — | — | — | — | — | 49.03 | — |
| | P-8 | | — | — | — | — | — | — | 49.03 |
| | P-9 | | — | — | — | — | — | — | — |
| | P-10 | | — | — | — | — | — | — | — |
| | P-11 | | — | — | — | — | — | — | — |
| | P-12 | | — | — | — | — | — | — | — |
| | P-13 | | — | — | — | — | — | — | — |
| | P-14 | | — | — | — | — | — | — | — |
| | P-15 | | — | — | — | — | — | — | — |
| | P-16 | | — | — | — | — | — | — | — |
| | P-17 | | — | — | — | — | — | — | — |
| | P-18 | | — | — | — | — | — | — | — |
| | P-19 | | — | — | — | — | — | — | — |
| Photopoly- | 1-[9-ethyl-6-(2-methylbenzoyl)-9H-carbazol-3-yl]ethanone-1- | | 0.37 | 0.37 | 0.37 | 0.37 | 0.37 | 0.37 | 0.37 |
| merization | (O-acetyloxime) (OXE-02, manufactured by BASF SE) | | | | | | | | |
| initiator | OXE-03, manufactured by BASF SE | | — | — | — | — | — | — | — |
| | 2-methyl-1-(4-methylthiophenyl)-2-morpholinopropan-1-one | | 0.74 | 0.74 | 0.74 | 0.74 | 0.74 | 0.74 | 0.74 |
| | (Irgacure907, manufactured by BASF SE) | | | | | | | | |
| | 1-(biphenyl-4-yl)-2-methyl-2-morpholinopropan-1-one (APi- | | — | — | — | — | — | — | — |
| | 307, manufactured by Shenzhen UV-ChemTech Co., Ltd.) | | | | | | | | |
| Blocked | Q-1 | 5.40 | 2.97 | 2.97 | 2.97 | 2.97 | 2.97 | 2.97 | 2.97 |
| isocyanate | Q-1-A | 5.40 | | | | | | | |
| compound | Q-1-B | 5.40 | | | | | | | |
| | Q-2 | 5.80 | | | | | | | |
| | Q-3 | 5.50 | | | | | | | |
| | Q-4 | 5.70 | | | | | | | |
| | Q-5 | 4.70 | | | | | | | |
| | Q-6 | 5.20 | | | | | | | |
| | Q-7 | 4.60 | | | | | | | |
| | Q-8 | 3.90 | 12.50 | 12.50 | 12.50 | 12.50 | 12.50 | 12.50 | 12.50 |

| | | | | | | | | |
|---|---|---|---|---|---|---|---|---|
| Additive | N-phenylglycine (manufactured by Tokyo Chemical Industry | 0.10 | 0.10 | 0.10 | 0.10 | 0.10 | 0.10 | 0.10 |
| | Co., Ltd.) | | | | | | | |
| | 1,2,4-triazole (manufactured by Otsuka Chemical Co., Ltd.) | — | — | — | — | — | — | — |
| | Benzoimidazole (manufactured by Tokyo Chemical Industry | 0.13 | 0.13 | 0.13 | 0.13 | 0.13 | 0.13 | 0.13 |
| | Co., Ltd.) | | | | | | | |
| | 5-amino-1H-tetrazole (manufactured by Tokyo Chemical | — | — | — | — | — | — | — |
| | Industry Co., Ltd.) | | | | | | | |
| | Isonicotinamide (manufactured by Tokyo Chemical Industry | 0.52 | 0.52 | 0.52 | 0.52 | 0.52 | 0.52 | 0.52 |
| | Co., Ltd.) | | | | | | | |
| | SMA EF-40 (manufactured by TOMOEGAWA CO., LTD.) | 1.20 | 1.20 | 1.20 | 1.20 | 1.20 | 1.20 | 1.20 |
| | MEGAFACE F551A (manufactured by DIC Corporation) | 0.19 | 0.19 | 0.19 | 0.19 | 0.19 | 0.19 | 0.19 |
| | MEGAFACE R-41 (manufactured by DIC Corporation) | | | | | | | |
| | DOWSIL 8032 ADDITIVE (manufactured by Dow Corning | | | | | | | |
| | Toray Co., Ltd.) | | | | | | | |
| | Ftergent 710FL (manufactured by Neos Corporation) | | | | | | | |

| | | | | | | | |
|---|---|---|---|---|---|---|---|
| Concentration of solid contents of coating liquid | 25% | 25% | 25% | 25% | 25% | 25% | 25% |

TABLE 5
Table 2 (4)

| | | | A-22 | A-23 | A-24 | A-25 | A-26 | A-27 |
|---|---|---|---|---|---|---|---|---|
| Polymerizable | Tricyclodecane dimethanol diacrylate (A-DCP, manufactured | | 18.26 | 18.26 | 18.26 | 18.26 | 18.26 | 18.26 |
| compound | by Shin-Nakamura Chemical Co., Ltd.) | | | | | | | |
| | Monomer having carboxy group ARONIX TO-2349 | | 3.04 | 3.04 | 3.04 | 3.04 | 3.04 | 3.04 |
| | (manufactured by Toagosei Co., Ltd.) | | | | | | | |
| | Urethane acrylate 8UX-015A (manufactured by Taisei Fine | | — | — | — | — | — | — |
| | Chemical Co., Ltd.) | | | | | | | |
| | A-NOD-N (manufactured by Shin-Nakamura Chemical Co., Ltd.) | | 2.79 | 2.79 | 2.79 | 2.79 | 2.79 | 2.79 |
| | A-DPH (manufactured by Shin-Nakamura Chemical Co., Ltd.) | | 8.15 | 8.15 | 8.15 | 8.15 | 8.15 | 8.15 |
| Alkali-soluble | P-1 | | — | — | — | — | — | — |
| resin | P-2 | | — | — | — | — | — | — |
| | P-3 | | — | — | — | — | — | — |
| | P-4 | | — | — | — | — | — | — |
| | P-5 | | — | — | — | — | — | — |
| | P-6 | | — | — | — | — | — | — |
| | P-7 | | — | — | — | — | — | — |
| | P-8 | | — | — | — | — | — | — |
| | P-9 | | 49.03 | — | — | — | — | — |
| | P-10 | | — | 49.03 | — | — | — | — |
| | P-11 | | — | — | 49.03 | — | — | — |
| | P-12 | | — | — | — | 49.03 | — | — |
| | P-13 | | — | — | — | — | 49.03 | — |
| | P-14 | | — | — | — | — | — | 49.03 |
| | P-15 | | — | — | — | — | — | — |
| | P-16 | | — | — | — | — | — | — |
| | P-17 | | — | — | — | — | — | — |
| | P-18 | | — | — | — | — | — | — |
| | P-19 | | — | — | — | — | — | — |
| Photopoly- | 1-[9-ethyl-6-(2-methylbenzoyl)-9H-carbazol-3-yl]ethanone-1- | | 0.37 | 0.37 | 0.37 | 0.37 | 0.37 | 0.37 |
| merization | (O-acetyloxime) (OXE-02, manufactured by BASF SE) | | | | | | | |
| initiator | OXE-03, manufactured by BASF SE | | — | — | — | — | — | — |
| | 2-methyl-1-(4-methylthiophenyl)-2-morpholinopropan-1-one | | — | — | 0.74 | 0.74 | 0.74 | 0.74 |
| | (Irgacure907, manufactured by BASF SE) | | | | | | | |
| | 1-(biphenyl-4-yl)-2-methyl-2-morpholinopropan-1-one (APi- | | 0.74 | 0.74 | — | — | — | — |
| | 307, manufactured by Shenzhen UV-ChemTech Co., Ltd.) | | | | | | | |
| Blocked | Q-1 | 5.40 | 2.97 | 2.97 | 2.97 | 2.97 | 2.97 | 2.97 |
| isocyanate | Q-1-A | 5.40 | | | | | | |
| compound | Q-1-B | 5.40 | | | | | | |
| | Q-2 | 5.80 | | | | | | |
| | Q-3 | 5.50 | | | | | | |
| | Q-4 | 5.70 | | | | | | |
| | Q-5 | 4.70 | | | | | | |
| | Q-6 | 5.20 | | | | | | |
| | Q-7 | 4.60 | | | | | | |
| | Q-8 | 3.90 | 12.50 | 12.50 | 12.50 | 12.50 | 12.50 | 12.50 |

| | | | | | | | |
|---|---|---|---|---|---|---|---|
| Additive | N-phenylglycine (manufactured by Tokyo Chemical Industry Co., Ltd.) | 0.10 | 0.10 | 0.10 | 0.10 | 0.10 | 0.10 |
| | 1,2,4-triazole (manufactured by Otsuka Chemical Co., Ltd.) | — | — | — | — | — | — |
| | Benzoimidazole (manufactured by Tokyo Chemical Industry Co., Ltd.) | 0.13 | 0.13 | 0.13 | 0.13 | 0.13 | 0.13 |
| | 5-amino-1H-tetrazole (manufactured by Tokyo Chemical Industry Co., Ltd.) | — | — | — | — | — | — |
| | Isonicotinamide (manufactured by Tokyo Chemical Industry Co., Ltd.) | 0.52 | 0.52 | 0.52 | 0.52 | 0.52 | 0.52 |
| | SMA EF-40 (manufactured by TOMOEGAWA CO., LTD.) | 1.20 | 1.20 | 1.20 | 1.20 | 1.20 | 1.20 |
| | MEGAFACE F551A (manufactured by DIC Corporation) | 0.19 | 0.19 | 0.19 | 0.19 | 0.19 | 0.19 |
| | MEGAFACE R-41 (manufactured by DIC Corporation) | | | | | | |
| | DOWSIL 8032 ADDITIVE (manufactured by Dow Corning Toray Co., Ltd.) | | | | | | |
| | Ftergent 710FL (manufactured by Neos Corporation) | | | | | | |

| | | | | | | |
|---|---|---|---|---|---|---|
| Concentration of solid contents of coating liquid | 25% | 25% | 25% | 25% | 25% | 25% |

TABLE 6
Table 2 (5)

| | | | A-28 | A-29 | A-30 | A-31 | A-32 | A-33 |
|---|---|---|---|---|---|---|---|---|
| Polymerizable | Tricyclodecane dimethanol diacrylate (A-DCP, manufactured | | 18.26 | 18.26 | 18.26 | 18.26 | 17.90 | 17.90 |
| compound | by Shin-Nakamura Chemical Co., Ltd.) | | | | | | | |
| | Monomer having carboxy group ARONIX TO-2349 | | 3.04 | 3.04 | 3.04 | 3.04 | 2.98 | 2.98 |
| | (manufactured by Toagosei Co., Ltd.) | | | | | | | |
| | Urethane acrylate 8UX-015A (manufactured by Taisei Fine | | — | — | — | — | — | — |
| | Chemical Co., Ltd.) | | | | | | | |
| | A-NOD-N (manufactured by Shin-Nakamura Chemical Co., Ltd.) | | 2.79 | 2.79 | 2.79 | 2.79 | 2.73 | 2.73 |
| | A-DPH (manufactured by Shin-Nakamura Chemical Co., Ltd.) | | 8.15 | 8.15 | 8.15 | 8.15 | 7.99 | 7.99 |
| Alkali-soluble | P-1 | | — | — | — | — | — | — |
| resin | P-2 | | — | — | — | — | — | — |
| | P-3 | | — | — | — | — | — | — |
| | P-4 | | — | — | — | — | — | — |
| | P-5 | | — | — | — | — | — | — |
| | P-6 | | — | — | — | — | — | — |
| | P-7 | | — | — | — | — | — | — |
| | P-8 | | — | — | — | — | — | — |
| | P-9 | | — | — | — | — | — | — |
| | P-10 | | — | — | — | — | — | — |
| | P-11 | | — | — | — | — | 52.67 | 52.67 |
| | P-12 | | — | — | — | — | — | — |
| | P-13 | | — | — | — | — | — | — |
| | P-14 | | — | — | — | — | — | — |
| | P-15 | | 49.03 | — | — | — | — | — |
| | P-16 | | — | 49.03 | — | — | — | — |
| | P-17 | | — | — | 49.03 | — | — | — |
| | P-18 | | — | — | — | 49.03 | — | — |
| | P-19 | | — | — | — | — | — | — |
| Photopoly- | 1-[9-ethyl-6-(2-methylbenzoyl)-9H-carbazol-3-yl]ethanone-1- | | 0.37 | 0.37 | 0.37 | 0.37 | 0.36 | 0.36 |
| merization | (O-acetyloxime) (OXE-02, manufactured by BASF SE) | | | | | | | |
| initiator | OXE-03, manufactured by BASF SE | | — | — | — | — | — | — |
| | 2-methyl-1-(4-methylthiophenyl)-2-morpholinopropan-1-one | | 0.74 | 0.74 | 0.74 | 0.74 | 0.73 | 0.73 |
| | (Irgacure907, manufactured by BASF SE) | | | | | | | |
| | 1-(biphenyl-4-yl)-2-methyl-2-morpholinopropan-1-one (APi- | | — | — | — | — | — | — |
| | 307, manufactured by Shenzhen UV-ChemTech Co., Ltd.) | | | | | | | |
| Blocked | Q-1 | 5.40 | 2.97 | 2.97 | 2.97 | 2.97 | | |
| isocyanate | Q-1-A | 5.40 | | | | | 12.50 | |
| compound | Q-1-B | 5.40 | | | | | | 12.50 |
| | Q-2 | 5.80 | | | | | | |
| | Q-3 | 5.50 | | | | | | |
| | Q-4 | 5.70 | | | | | | |
| | Q-5 | 4.70 | | | | | | |
| | Q-6 | 5.20 | | | | | | |
| | Q-7 | 4.60 | | | | | | |
| | Q-8 | 3.90 | 12.50 | 12.50 | 12.50 | 12.50 | | |

| | | | | | | | |
|---|---|---|---|---|---|---|---|
| Additive | N-phenylglycine (manufactured by Tokyo Chemical Industry Co., Ltd.) | 0.10 | 0.10 | 0.10 | 0.10 | 0.10 | 0.10 |
| | 1,2,4-triazole (manufactured by Otsuka Chemical Co., Ltd.) | — | — | — | — | — | — |
| | Benzoimidazole (manufactured by Tokyo Chemical Industry Co., Ltd.) | 0.13 | 0.13 | 0.13 | 0.13 | 0.13 | 0.13 |
| | 5-amino-1H-tetrazole (manufactured by Tokyo Chemical Industry Co., Ltd.) | — | — | — | — | — | — |
| | Isonicotinamide (manufactured by Tokyo Chemical Industry Co., Ltd.) | 0.52 | 0.52 | 0.52 | 0.52 | 0.52 | 0.52 |
| | SMA EF-40 (manufactured by TOMOEGAWA CO., LTD.) | 1.20 | 1.20 | 1.20 | 1.20 | 1.20 | 1.20 |
| | MEGAFACE F551A (manufactured by DIC Corporation) | 0.19 | 0.19 | 0.19 | 0.19 | 0.19 | 0.19 |
| | MEGAFACE R-41 (manufactured by DIC Corporation) | | | | | | |
| | DOWSIL 8032 ADDITIVE (manufactured by Dow Corning Toray Co., Ltd.) | | | | | | |
| | Ftergent 710FL (manufactured by Neos Corporation) | | | | | | |

| | | | | | | |
|---|---|---|---|---|---|---|
| Concentration of solid contents of coating liquid | 25% | 25% | 25% | 25% | 20% | 20% |

TABLE 7
Table 2 (6)

| | | | A-34 | A-35 | A-36 | A-37 | A-38 | A-39 | A-40 | A-41 | A′-1 |
|---|---|---|---|---|---|---|---|---|---|---|---|
| Polymerizable | Tricyclodecane dimethanol diacrylate | | 18.00 | 17.00 | 19.00 | 19.00 | 19.00 | 18.30 | 18.30 | 19.00 | 17.90 |
| compound | (A-DCP, manufactured by Shin- | | | | | | | | | | |
| | Nakamura Chemical Co., Ltd.) | | | | | | | | | | |
| | Monomer having carboxy group | | 3.00 | 2.00 | 3.00 | 3.00 | 3.00 | 3.00 | 3.00 | 3.00 | 2.98 |
| | ARONIX TO-2349 (manufactured | | | | | | | | | | |
| | by Toagosei Co., Ltd.) | | | | | | | | | | |
| | Urethane acrylate 8UX-015A | | 11.00 | 5.00 | 3.00 | | | — | — | | — |
| | (manufactured by Taisei Fine | | | | | | | | | | |
| | Chemical Co., Ltd.) | | | | | | | | | | |
| | A-NOD-N (manufactured by Shin- | | — | — | 5.00 | 5.00 | 5.00 | 2.80 | 2.80 | 5.00 | 2.73 |
| | Nakamura Chemical Co., Ltd.) | | | | | | | | | | |
| | A-DPH (manufactured by Shin- | | 1.00 | — | 9.00 | 9.00 | 9.00 | 8.00 | 8.00 | 9.00 | 7.99 |
| | Nakamura Chemical Co., Ltd.) | | | | | | | | | | |
| Alkali-soluble | P-1 | | — | — | — | — | — | 49.00 | 49.00 | — | 52.67 |
| resin | P-2 | | — | — | — | — | — | — | — | — | — |
| | P-3 | | — | — | — | — | — | — | — | — | — |
| | P-4 | | — | — | — | — | — | — | — | — | — |
| | P-5 | | — | — | — | — | — | — | — | — | — |
| | P-6 | | — | — | — | — | — | — | — | — | — |
| | P-7 | | — | — | — | — | — | — | — | — | — |
| | P-8 | | — | — | — | — | — | — | — | — | — |
| | P-9 | | — | — | — | — | — | — | — | — | — |
| | P-10 | | — | — | — | — | — | — | — | — | — |
| | P-11 | | — | — | — | — | — | — | — | — | — |
| | P-12 | | — | — | — | — | — | — | — | — | — |
| | P-13 | | — | — | — | — | — | — | — | — | — |
| | P-14 | | — | — | — | — | — | — | — | — | — |
| | P-15 | | — | — | — | — | — | — | — | — | — |
| | P-16 | | — | — | — | — | — | — | — | — | — |
| | P-17 | | — | — | — | — | — | — | — | — | — |
| | P-18 | | — | — | — | — | — | — | — | — | — |
| | P-19 | | 51.70 | 55.80 | 45.60 | 48.60 | 48.60 | — | — | 48.60 | — |
| Photopoly- | 1-[9-ethyl-6-(2-methylbenzoyl)-9H- | | 0.50 | 0.40 | 0.60 | 0.50 | 0.60 | 0.40 | 0.50 | 0.60 | 0.36 |
| merization | carbazol-3-yl]ethanone-1-(O- | | | | | | | | | | |
| initiator | acetyloxime) (OXE-02, manufactured | | | | | | | | | | |
| | by BASF SE) | | | | | | | | | | |
| | OXE-03, manufactured by BASF SE | | | | | | | — | — | | — |
| | 2-methyl-1-(4-methylthiophenyl)-2- | | 1.00 | 1.00 | 1.00 | 1.00 | 1.00 | — | — | 1.00 | 0.73 |
| | morpholinopropan-1-one (Irgacure907, | | | | | | | | | | |
| | manufactured by BASF SE) | | | | | | | | | | |
| | 1-(biphenyl-4-yl)-2-methyl-2- | | — | — | — | — | — | 0.80 | 0.80 | — | — |
| | morpholinopropan-1-one (APi-307, | | | | | | | | | | |
| | manufactured by Shenzhen UV- | | | | | | | | | | |
| | ChemTech Co., Ltd.) | | | | | | | | | | |
| Blocked | Q-1 | 5.40 | 9.00 | 6.00 | | 12.00 | | 2.97 | 2.87 | | |
| isocyanate | Q-1-A | 5.40 | | | 6.00 | | 6.00 | | | 6.00 | |
| compound | Q-1-B | 5.40 | | | 6.00 | | 6.00 | | | 6.00 | |
| | Q-2 | 5.80 | | | | | | | | | |
| | Q-3 | 5.50 | 3.00 | | | | | | | | |
| | Q-4 | 5.70 | | | | | | | | | |
| | Q-5 | 4.70 | | | | | | | | | |
| | Q-6 | 5.20 | | | | | | | | | |
| | Q-7 | 4.60 | | | | | | | | | |
| | Q-8 | 3.90 | | 11.00 | | | | 12.50 | 12.50 | | 12.50 |

| | | | | | | | | | | |
|---|---|---|---|---|---|---|---|---|---|---|
| Additive | N-phenylglycine (manufactured by | 0.12 | 0.12 | 0.12 | 0.10 | 0.10 | 0.10 | 0.10 | 0.10 | 0.10 |
| | Tokyo Chemical Industry Co., Ltd.) | | | | | | | | | |
| | 1,2,4-triazole (manufactured by | 0.10 | 0.10 | 0.10 | 0.20 | — | — | — | — | — |
| | Otsuka Chemical Co., Ltd.) | | | | | | | | | |
| | Benzoimidazole (manufactured by | 0.10 | 0.10 | 0.10 | — | 0.20 | 0.13 | 0.13 | 0.20 | 0.13 |
| | Tokyo Chemical Industry Co., Ltd.) | | | | | | | | | |
| | 5-amino-1H-tetrazole (manufactured | — | — | — | — | — | — | — | — | — |
| | by Tokyo Chemical Industry Co., Ltd.) | | | | | | | | | |
| | Isonicotinamide (manufactured by | 0.40 | 0.40 | 0.40 | 0.40 | 0.40 | 0.52 | 0.52 | 0.40 | 0.52 |
| | Tokyo Chemical Industry Co., Ltd.) | | | | | | | | | |
| | SMA EF-40 (manufactured by | 1.00 | 1.00 | 1.00 | 1.00 | 1.00 | 1.20 | 1.20 | 1.00 | 1.20 |
| | TOMOEGAWA CO., LTD.) | | | | | | | | | |
| | MEGAFACE F551A (manufactured | 0.10 | 0.10 | 0.10 | | | 0.27 | | | 0.19 |
| | by DIC Corporation) | | | | | | | | | |
| | MEGAFACE R-41 (manufactured by | | | | 0.20 | | | | | |
| | DIC Corporation) | | | | | | | | | |
| | DOWSIL 8032 ADDITIVE | | | | | | | 0.27 | 0.10 | |
| | (manufactured by Dow Corning | | | | | | | | | |
| | Toray Co., Ltd.) | | | | | | | | | |
| | Ftergent 710FL (manufactured by | | | | | 0.10 | | | | |
| | Neos Corporation) | | | | | | | | | |

| | | | | | | | | | |
|---|---|---|---|---|---|---|---|---|---|
| Concentration of solid contents of coating liquid | 20% | 20% | 20% | 20% | 20% | 25% | 25% | 20% | 25% |

<Preparation of Coating Liquid for Forming Refractive Index-Adjusting Layer>

Next, a coating liquid B-1 for forming a refractive index-adjusting layer was prepared with a composition shown in Table 3. The numerical values in Table 3 represent “parts by mass”

TABLE 8
Table 3

| | | B-1 |
|---|---|---|
| Nano-use OZS-30M: ZrO₂ particles (containing tin oxide) | | 4.34 |
| methanol dispersion liquid (non-volatile amount: 30.5%), | | |
| manufactured by Nissan Chemical Corporation | | |
| Ammonia water (25%) | | 7.84 |
| Binder polymer | Copolymer resin of methacrylic acid/aryl | 0.20 |
| | methacrylate (Mw: 38,000, composition ratio = | |
| | 20/80 (% by mass)) | |
| | ARUFON UC-3920 | 0.02 |
| | (manufactured by Toagosei Co., Ltd.) | |

| | |
|---|---|
| Monomer having carboxy group | 0.03 |
| ARONIX TO-2349 (manufactured by Toagosei Co., Ltd.) | |
| Adenine (manufactured by Tokyo Chemical Industry Co., Ltd.) | 0.03 |
| N-methyldiethanolamine (manufactured by Tokyo | 0.03 |
| Chemical Industry Co., Ltd.) | |
| MEGAFACE F444 (manufactured by DIC Corporation) | 0.01 |
| Ion exchange water | 21.3 |
| Methanol | 66.2 |
| Total (part by mass) | 100 |

### Production of Transfer Films of Examples 1 to 45 and Comparative Example 1

Any one of the photosensitive compositions A-1 to A-38 and A′-1 was applied onto LUMIRROR 16KS40 (thickness: 16 μm, manufactured by Toray Industries, Inc., polyethylene terephthalate film) which is a temporary support using a slit-shaped nozzle, and the solvent was volatilized in a drying zone at 100° C. to form a photosensitive composition layer on the temporary support. The coating amount of the photosensitive composition was adjusted to be the thickness of the photosensitive composition layer shown in Table 4. Next, a protective film (LUMIRROR 16KS40 (manufactured by Toray Industries, Inc.)) was pressure-bonded to the photosensitive composition layer to produce transfer films of Examples 1 to 45 and Comparative Example 1.

<Production of Laminate>

A cycloolefin resin film having a film thickness of 38 μm and a refractive index of 1.53 was subjected to a corona discharge treatment for 3 seconds under the conditions of an electrode length of 240 mm, a distance between work electrodes of 1.5 mm at an output voltage of 100% and an output of 250 W with a wire electrode having a diameter of 1.2 mm by using a high frequency oscillator, to perform the surface reforming. The obtained film was used as a transparent substrate.

Next, a material of a material-C shown in Table 4 was coated on the transparent substrate using a slit-shaped nozzle, irradiated with ultraviolet rays (integrated light amount of 300 mJ/cm²), and dried at approximately 110° C. to form a transparent film having a refractive index of 1.60 and a film thickness of 80 nm.

TABLE 4

| Raw material | Material-C |
|---|---|
| ZrO₂: ZR-010 manufactured by OLAR CO., LTD. | 2.08 |
| DPHA liquid (dipentaerythritol hexaacrylate: 38%, dipentaerythritol | 0.29 |
| pentaacrylate: 38%, 1-methoxy-2-propylacetate: 24%) | |
| Urethane-based monomer: UK oligo UA-32P manufactured by | 0.14 |
| Shin-Nakamura Chemical Co., Ltd.: non-volatile amount: 75%, | |
| 1-methoxy-2-propylacetate: 25%) | |
| Monomer mixture (polymerizable compound (b2-1) described in paragraph | 0.36 |
| [0111] of JP2012-78528A, n = 1: content of tripentaerythritol octaacrylate: | |
| 85%, total of n = 2 and n = 2 as impurities: 15%) | |
| Polymer solution 1 (structural formula P-25 described in paragraph [0058] of | 1.89 |
| JP2008-146018A: weight-average molecular weight = 35,000, solid content: | |
| 45%, 1-methoxy-2-propylacetate: 15%, 1-methoxy-2-propanol: 40%) | |
| Photoradical polymerization initiator: | 0.03 |
| 2-benzyl-2-dimethylamino-1-(4-morpholinophenyl)-butanone [Irgacure | |
| (resitered trademark) 379, manufactured by BASF SE] | |
| Photopolymerization initiator: KAYACURE DETX-S (Nippon Kayaku | 0.03 |
| Co., Ltd., alkylthioxanthone) | |
| Polymer solution 2 (polymer having structural formula represented by Formula | 0.01 |
| (3): solution having weight-average molecular weight of 15000, non-volatile | |
| amount: 30% by mass, methyl ethyl ketone: 70% by mass) | |
| 1-methoxy-2-propylacetate | 38.73 |
| Methyl ethyl ketone | 56.80 |
| Total (part by mass) | 100 |

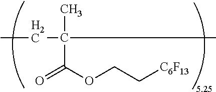

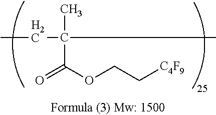

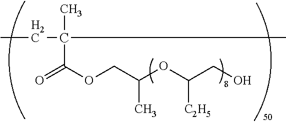

Next, the ITO thin film was etched and patterned by a known chemical etching method to obtain a conductive substrate having a transparent film and a transparent electrode part on the transparent substrate.

The protective film of each transfer film of Examples and Comparative Example was peeled off, the surface of the exposed photosensitive composition layer was brought into contact with the transparent electrode part of the conductive substrate and laminated so that the photosensitive composition layer covered (was pressure-bonded to) the transparent electrode part to form a laminate in which the photosensitive composition layer and the temporary support were arranged on the conductive substrate.

The above-described lamination was performed under the conditions in which a temperature of the transparent substrate was 40° C., a rubber roller temperature was 100° C., a linear pressure was 3 N/cm, and a transportation speed was 2 m/min, by using a vacuum laminator manufactured by MCK Co., Ltd.

Thereafter, using a proximity type exposure machine (manufactured by Hitachi High-Tech Electronics Engineering Co., Ltd.) having an ultra-high pressure mercury lamp, a surface of an exposure mask (quartz exposure mask having a pattern for forming an overcoat) and the temporary support were closely attached, and the laminate was exposed in a patterned shape with an exposure amount of 120 mJ/cm² (measured value with i-line) through the temporary support. A main wavelength of the exposure light at the time of irradiation was from light at a wavelength of 365 nm.

The above-described exposed sample was allowed to stand in an environment of 23° C. and 55% for 48 hours, the temporary support was peeled off, and the sample was developed with a 1% sodium carbonate aqueous solution at 32° C. for 60 seconds. Thereafter, the residue was removed by spraying ultrapure water from an ultra-high pressure washing nozzle onto the transparent substrate after the development treatment. Subsequently, air was blown to remove water on the transparent substrate.

Next, the obtained pattern was exposed with an exposure amount of 400 mJ/cm² (measured value with i-line) using a post-exposure machine (manufactured by Ushio, Inc.) having a high pressure mercury lamp (post-exposure).

Thereafter, the pattern was subjected to a post-baking treatment at 145° C. for 30 minutes to form a laminate having the transparent film, the transparent electrode part, and the pattern (a cured film of a photosensitive composition layer) in this order on the transparent substrate.

<Evaluation of Corrosiveness>

Using the transfer film of each of Examples and Comparative Example, in which the protective film had been peeled off, in the same manner as in the method of transferring to a film in which a transparent film and a transparent electrode part were formed on a transparent substrate, on a polyethylene terephthalate (PET) film (manufactured by GEOMATEC Co., Ltd.) laminated with a copper foil (substitution for an electrode in a capacitive input device), the surface of the exposed photosensitive composition is brought into contact with the copper foil on the PET film, the photosensitive composition layer was laminated (affixed) so as to cover the copper foil, and post-processes (peeling of the temporary support, exposure, development, post-baking, and the like) were carried out to obtain a sample (laminate) having the copper foil and a pattern (cured film of the photosensitive composition layer) in this order on the PET film.

5 cm³ of salt water having a concentration of 50 g/L was dropped onto the surface of the sample pattern and spread uniformly to 50 cm², water was volatilized at normal temperature, and using a HAST test device EHS-221MD (manufactured by ESPEC Corp.), the sample was allowed to pass in an environment of 110° C. and 85% for 32 hours. Thereafter, the salt water was wiped off, and the surface condition of the sample was observed to perform evaluation according to the following grades.

AA, A, B, and C are levels which are practically necessary, and AA is preferable.

(Evaluation Standard)

AA: no discoloration of copper was observed.

A: slight discoloration of copper was observed in a part.

B: light discoloration of copper was observed in a part.

C: light discoloration of copper was observed on the entire surface.

D: discoloration of copper was remarkably observed on the entire surface.

<Evaluation of Development Residue>

The developed-removed portion of the above-described laminate was visually observed and observed with an optical microscope (objective: 20×).

A or B is a practical level, and A is preferable.

(Evaluation Standard)

A: residue could not be visually recognized even in a case of being observed with an optical microscope.

B: residues were observed in a small part by the observation with an optical microscope.

C: residues were clearly generated on the entire surface by the visual observation.

The evaluation results are summarized in Table 5.

TABLE 10
Table 5 (1)

| | | | | | | | | Mass ratio of blocked |
|---|---|---|---|---|---|---|---|---|
| | | | | | | | | isocyanate compound |
| | | | | | | Second blocked [symbol] | | (first blocked |
| | Photosensitive | First blocked [symbol] compound | | | | compound | | iso [symbol] yanate compound/ |
| | composition | | NCO value | | NCO value | | NCO value | second blocked |
| | Type | Type | mmol/g | Type | mmol/g | Type | mmol/g | isocyanate compound) |
| Example 1 | A-1 | Compound Q-1 | 5.4 | | | — | — | — |
| Example 2 | A-2 | Compound Q-2 | 5.8 | | | — | — | — |
| Example 3 | A-3 | Compound Q-3 | 5.5 | | | — | — | — |
| Example 4 | A-4 | Compound Q-4 | 5.7 | | | — | — | — |
| Example 5 | A-5 | Compound Q-5 | 4.7 | | | — | — | — |
| Example 6 | A-6 | Compound Q-6 | 5.2 | | | — | — | — |
| Example 7 | A-7 | Compound Q-7 | 4.6 | | | — | — | — |
| Example 8 | A-8 | Compound Q-1 | 5.4 | | | Compound Q-8 | 3.9 | 0.15 |
| Example 9 | A-9 | Compound Q-1 | 5.4 | | | Compound Q-8 | 3.9 | 0.25 |
| Example 10 | A-10 | Compound Q-1 | 5.4 | | | Compound Q-8 | 3.9 | 0.5 |
| Example 11 | A-11 | Compound Q-3 | 5.5 | | | Compound Q-8 | 3.9 | 0.7 |
| Example 12 | A-12 | Compound Q-4 | 5.7 | | | Compound Q-8 | 3.9 | 0.4 |
| Example 13 | A-13 | Compound Q-5 | 4.7 | | | Compound Q-8 | 3.9 | 0.2 |
| Example 14 | A-14 | Compound Q-3 | 5.5 | Compound Q-1 | 5.4 | — | — | — |
| Example 15 | A-15 | Compound Q-1 | 5.4 | | | Compound Q-8 | 3.9 | 0.25 |
| Example 16 | A-16 | Compound Q-1 | 5.4 | | | Compound Q-8 | 3.9 | 0.25 |
| Example 17 | A-17 | Compound Q-1 | 5.4 | | | Compound Q-8 | 3.9 | 0.25 |
| Example 18 | A-18 | Compound Q-1 | 5.4 | | | Compound Q-8 | 3.9 | 0.25 |
| Example 19 | A-19 | Compound Q-1 | 5.4 | | | Compound Q-8 | 3.9 | 0.25 |
| Example 20 | A-20 | Compound Q-1 | 5.4 | | | Compound Q-8 | 3.9 | 0.25 |
| Example 21 | A-21 | Compound Q-1 | 5.4 | | | Compound Q-8 | 3.9 | 0.25 |

| | Alkali-soluble resin | | NCO value of | | | |
|---|---|---|---|---|---|---|
| | | Content of | photosensitive | Thickness of | | |
| | | vinylbenzene | composition | photosensitive | | |
| | | derivative | layer | composition | | Development |
| | Type | (% by mass) | mmol/g | layer | Corrosiveness | residue |
| Example 1 | P-11 | 48 | 0.675 | 5 μm | AA | B |
| Example 2 | P-11 | 48 | 0.723 | 5 μm | A | B |
| Example 3 | P-11 | 48 | 0.688 | 5 μm | AA | B |
| Example 4 | P-11 | 48 | 0.713 | 5 μm | AA | B |
| Example 5 | P-11 | 48 | 0.5 [symbol] 8 | 5 μm | A | B |
| Example 6 | P-11 | 48 | 0.650 | 5 μm | A | A |
| Example 7 | P-11 | 48 | 0.575 | 5 μm | A | B |
| Example 8 | P-1 | 30 | 0.634 | 5 μm | B | A |
| Example 9 | P-1 | 30 | 0.650 | 5 μm | B | A |
| Example 10 | P-1 | 30 | 0.681 | 5 μm | B | A |
| Example 11 | P-1 | 30 | 0.705 | 5 μm | B | A |
| Example 12 | P-1 | 30 | 0.683 | 5 μm | B | A |
| Example 13 | P-1 | 30 | 0.624 | 5 μm | C | A |
| Example 14 | P-1 | 30 | 0.6 [symbol] | 5 μm | B | B |
| Example 15 | P-2 | 35 | 0.648 | 5 μm | A | A |
| Example 16 | P-3 | 40 | 0.648 | 5 μm | A | A |
| Example 17 | P-4 | 45 | 0.648 | 5 μm | AA | A |
| Example 18 | P-5 | 50 | 0.648 | 5 μm | AA | A |
| Example 19 | P-6 | 5 [symbol] | 0.648 | 5 μm | AA | B |
| Example 20 | P-7 | 49 | 0.648 | 5 μm | AA | A |
| Example 21 | P-8 | 48 | 0.648 | 5 μm | AA | A |

[symbol]

indicates data missing or illegible when filed

TABLE 11
Table 5 (2)

| | | | | | | Second blocked iso [symbol] y [symbol] t [symbol] | |
|---|---|---|---|---|---|---|---|
| | Photosensitive | First blocked isocyanate compound | | | | compound | |
| | composition | | NCO value | | NCO value | | NCO value |
| | Type | Type | mmol/g | Type | mmol/g | Type | mmol/g |
| Example 22 | A-22 | Compound Q-1 | [symbol] .4 | | | Compound Q-8 | 3.9 |
| Example 23 | A-23 | Compound Q-1 | [symbol] .4 | | | Compound Q-8 | 3.9 |
| Example 24 | A-2 [symbol] | Compound Q-1 | [symbol] .4 | | | Compound Q-8 | 3.9 |
| Example 25 | A-25 | Compound Q-1 | [symbol] .4 | | | Compound Q-8 | 3.9 |
| Example 26 | A-26 | Compound Q-1 | [symbol] .4 | | | Compound Q-8 | 3.9 |
| Example 27 | A-27 | Compound Q-1 | [symbol] .4 | | | Compound Q-8 | 3.9 |
| Example 28 | A- [symbol] 8 | Compound Q-1 | [symbol] .4 | | | Compound Q-8 | 3.9 |
| Example 29 | A-29 | Compound Q-1 | [symbol] .4 | | | Compound Q-8 | 3.9 |
| Example 30 | A-30 | Compound Q-1 | [symbol] .4 | | | Compound Q-8 | 3.9 |
| Example 31 | A-31 | Compound Q-1 | [symbol] .4 | | | Compound Q-8 | 3.9 |
| Example 32 | A-23 | Compound Q-1 | [symbol] .4 | | | Compound Q-8 | 3.9 |
| Example 33 | A-23 | Compound Q-1 | [symbol] .4 | | | Compound Q-8 | 3.9 |
| Example 34 | A-24 | Compound Q-1 | [symbol] .4 | | | Compound Q-8 | 3.9 |
| Example 35 | A-25 | Compound Q-1 | [symbol] .4 | | | Compound Q-8 | 3.9 |
| Example 36 | A-32 | Compound Q-1-A | [symbol] .4 | | | — | — |
| Example 37 | A-33 | Compound Q-1-B | [symbol] .4 | | | — | — |
| Example 38 | A-34 | Compound Q-1 | [symbol] .4 | Compound Q-3 | 5. [symbol] | — | — |
| Example 39 | A-35 | Compound Q-1 | [symbol] .4 | | | Compound Q-8 | 3.9 |
| Example 40 | A-36 | Compound Q-1-A | [symbol] .4 | Compound Q-1-B | 5.4 | — | — |
| Example 41 | A-37 | Compound Q-1 | [symbol] .4 | | | — | — |
| Example 42 | A-38 | Compound Q-1-A | [symbol] .4 | Compound Q-1-B | 5.4 | — | — |
| Example 43 | A-39 | Compound Q-1 | [symbol] .4 | | | Compound Q-8 | 3.9 |
| Example 44 | A-40 | Compound Q-1 | [symbol] .4 | | | Compound Q-8 | 3.9 |
| Example 45 | A-41 | Compound Q-1-A | [symbol] .4 | Compound Q-1-B | 5.4 | — | — |
| Comparative | A′-1 | — | — | — | — | Compound Q-8 | 3.9 |
| Example 1 | | | | | | | |

| | Mass [symbol] io of blocked | Alkali-soluble resin | | NCO value of | | | |
|---|---|---|---|---|---|---|---|
| | i [symbol] compound | | Con [symbol] of | photosensitive | Thickness of | | |
| | ( [symbol] blocked iso [symbol] y [symbol] e | | vinylbenzene | composition | photosensitive | | |
| | compound/second blocked | | derivative | layer | composition | | Dev [symbol] |
| | i [symbol] y [symbol] e compound) | Type | (% by mass) | mmol/g | layer | Corrosiveness | residue |
| Example 22 | 0.25 | P- [symbol] | 4 [symbol] | 0.648 | 5 μm | AA | A |
| Example 23 | 0.25 | P-10 | 4 [symbol] | 0.648 | 5 μm | AA | A |
| Example 24 | 0.25 | P-11 | 48 | 0.648 | 5 μm | AA | A |
| Example 25 | 0.25 | P-12 | 48 | 0.648 | 5 μm | AA | A |
| Example 26 | 0.25 | P-13 | 50 | 0.648 | 5 μm | AA | A |
| Example 27 | 0.25 | P-14 | 51 | 0.648 | 5 μm | AA | A |
| Example 28 | 0.25 | P-15 | 47 | 0.648 | 5 μm | AA | A |
| Example 29 | 0.25 | P-16 | 4 [symbol] | 0.648 | 5 μm | AA | A |
| Example 30 | 0.25 | P-17 | 45 | 0.648 | 5 μm | AA | A |
| Example 31 | 0.25 | P-18 | 50 | 0.648 | 5 μm | AA | A |
| Example 32 | 0.25 | P-9 | 4 [symbol] | 0.648 | 2 μm | C | A |
| Example 33 | 0.25 | P-10 | 4 [symbol] | 0.648 | 3 μm | B | A |
| Example 34 | 0.25 | P-11 | 48 | 0.648 | 4 μm | A | A |
| Example 35 | 0.25 | P-12 | 48 | 0.648 | 10 μm | AA | A |
| Example 36 | — | P-11 | 48 | 0.675 | 5 μm | AA | B |
| Example 37 | — | P-11 | 48 | 0.67 [symbol] | [symbol] μm | AA | B |
| Example 38 | — | P [symbol] | 0 | 0.651 | [symbol] μm | B | A |
| Example 39 | 0. [symbol] | P [symbol] | 0 | 0.753 | [symbol] μm | B | A |
| Example 40 | — | P [symbol] | 0 | 0.648 | [symbol] μm | B | A |
| Example 41 | — | P [symbol] | 0 | 0.648 | [symbol] μm | B | A |
| Example 42 | — | P [symbol] | 0 | 0.648 | [symbol] μm | B | A |
| Example 43 | 0.25 | P-1 | [symbol] 0 | 0.648 | [symbol] μm | AA | A |
| Example 44 | 0.25 | P-1 | [symbol] 0 | 0.648 | [symbol] μm | AA | A |
| Example 45 | — | P [symbol] | 0 | 0.648 | [symbol] μm | B | A |
| Comparative | — | P-1 | [symbol] 0 | 0. [symbol] 88 | [symbol] μm | D | A |
| Example 1 | | | | | | | |

[symbol]

indicates data missing or illegible when filed

As shown in Table 5, in a case where a photosensitive composition layer including the alkali-soluble resin, the polymerizable compound, the polymerization initiator, and the first blocked isocyanate compound was used, it was shown that the corrosion of the wiring line (electrode) could be suppressed (Examples 1 to 45).

In comparison with Examples 1 to 4 and 6, in a case where the first blocked isocyanate compound had a ring structure (Examples 1, 3, and 4), it was shown that the corrosion of the wiring line (electrode) could be further suppressed.

In comparison with Examples 1, 3 to 5, and 7, in a case where the NCO value of the first blocked isocyanate compound was 5.0 mmol/g or more (Examples 1, 3, and 4), it was shown that the corrosion of the wiring line (electrode) could be further suppressed.

In comparison with Examples 8 to 10 and 15 to 31, in a case where the content of the structural unit derived from a vinylbenzene derivative was 35% by mass or more with respect to the total amount of all structural units included in the above-described alkali-soluble resin (Examples 15 to 31), it was shown that the corrosion of the wiring line (electrode) could be further suppressed. In particular, in a case where the content of the structural unit derived from a vinylbenzene derivative was 45% by mass or more with respect to the total amount of all structural units included in the above-described alkali-soluble resin (Examples 17 to 31), it was shown that the corrosion of the wiring line (electrode) could be further suppressed.

In comparison with Examples 22 to 25 and 32 to 35, in a case where the thickness of the photosensitive composition layer (Examples 22 to 25 and 33 to 35) was 3 μm or more, it was shown that the corrosion of the wiring line (electrode) could be further suppressed.

On the other hand, in a case where a photosensitive composition layer including no first blocked isocyanate compound was used, it was shown that the corrosion of the wiring line (electrode) was remarkable (Comparative Example 1).

A transfer film having a refractive index-adjusting layer corresponding to each of Examples and Comparative Example was obtained in the same procedure as the above-described transfer film of each of Examples and Comparative Example, except that, in the production of the transfer film of each of Examples and Comparative Example, the coating liquid B-1 for forming a refractive index-adjusting layer was applied to the photosensitive composition layer to form a refractive index-adjusting layer (refractive index: 1.60 or more) having a thickness of 80 nm.

In a case where the above-described evaluations were performed using the transfer film having the refractive index-adjusting layer thus obtained, the evaluation results were the same as in the case where the transfer film of each of Examples and Comparative Example was used.

### EXPLANATION OF REFERENCES

18: metal conductive material protective film
32: substrate
56: lead wire
70: first metal conductive material
72: second metal conductive material
74: image display region
75: image non-display region
90: touch panel
112: first island-shaped electrode portion
114: second island-shaped electrode portion
116: first wiring part
118: second wiring part (bridge wire)
120: through hole
124: transparent substrate (transparent film substrate)
130: protective layer
132: overcoat layer
134: first electrode pattern
136: second electrode pattern
200: transparent laminate
P: extending direction of first electrode pattern
Q: extending direction of second electrode pattern

What is claimed is:

1. A transfer film comprising:
a temporary support; and
a photosensitive composition layer disposed on the temporary support,
wherein the photosensitive composition layer includes an alkali-soluble resin, a polymerizable compound, a polymerization initiator, and a blocked isocyanate compound having an NCO value of 4.5 mmol/g or more.

2. The transfer film according to claim 1,
wherein the NCO value of the blocked isocyanate compound is more than 5.0 mmol/g.

3. The transfer film according to claim 1,
wherein the blocked isocyanate compound has a ring structure.

4. The transfer film according to claim 1,
wherein the blocked isocyanate compound is a blocked isocyanate compound represented by Formula Q,
B¹-A¹-L¹-A²-B² Formula Q
in Formula Q, B¹ and B² each independently represent a blocked isocyanate group, A¹ and A² each independently represent a single bond or an alkylene group having 1 to 10 carbon atoms, and L¹ represents a divalent linking group.

5. The transfer film according to claim 1,
wherein the blocked isocyanate compound is a blocked isocyanate compound represented by Formula QA,
B^{1a}-A^{1a}-L^{1a}-A^{2a}-B^{2a} Formula QA
in Formula QA, B^{1a} and B^{2a} each independently represent a blocked isocyanate group, A^{1a} and A^{2a} each independently represent a divalent linking group, and L^{1a} represents a cyclic divalent saturated hydrocarbon group or a divalent aromatic hydrocarbon group.

6. The transfer film according to claim 1,
wherein the photosensitive composition layer further includes a blocked isocyanate compound having an NCO value of less than 4.5 mmol/g.

7. The transfer film according to claim 1,
wherein the alkali-soluble resin includes a structural unit derived from a vinylbenzene derivative, a structural unit having a radically polymerizable group, and a structural unit having an acid group, and
a content of the structural unit derived from the vinylbenzene derivative is 35% by mass or more with respect to a total amount of all structural units included in the alkali-soluble resin.

8. The transfer film according to claim 7,
wherein the content of the structural unit derived from the vinylbenzene derivative is 45% by mass or more with respect to the total amount of all structural units included in the alkali-soluble resin.

9. The transfer film according to claim 1, further comprising:
a refractive index-adjusting layer,
wherein the refractive index-adjusting layer is disposed in contact with the photosensitive composition layer, and
a refractive index of the refractive index-adjusting layer is 1.60 or more.

10. The transfer film according to claim 1,
wherein the photosensitive composition layer is used for forming a touch panel electrode protective film.

11. A method for producing a laminate, comprising:
an affixing step of bringing the photosensitive composition layer on the temporary support of the transfer film according to claim 1 into contact with a substrate having a conductive layer to affix the photosensitive composition layer to the substrate and obtain a photosensitive composition layer-attached substrate having the substrate, the conductive layer, the photosensitive composition layer, and the temporary support in this order;
an exposing step of exposing the photosensitive composition layer in a patterned manner; and
a developing step of developing the exposed photosensitive composition layer to form a pattern,
wherein the producing method further includes, between the affixing step and the exposing step or between the exposing step and the developing step, a peeling step of peeling the temporary support from the substrate with a photosensitive composition layer.

12. A transfer film comprising:
a temporary support; and
a photosensitive composition layer disposed on the temporary support,
wherein the photosensitive composition layer includes an alkali-soluble resin, a polymerizable compound, a polymerization initiator, and a blocked isocyanate compound, and
an NCO value of the photosensitive composition layer is more than 0.50 mmol/g.

13. A blocked isocyanate compound represented by Formula QA,
B^{1a}-A^{1a}-L^{1a}-A^{2a}-B^{2a} Formula QA
in Formula QA, B^{1a} and B^{2a} each independently represent a blocked isocyanate group, A^{1a} and A^{2a} each independently represent a divalent linking group, and L^{1a} represents a cyclic divalent saturated hydrocarbon group or a divalent aromatic hydrocarbon group.

14. The blocked isocyanate compound according to claim 13,
wherein the blocked isocyanate compound is represented by Formula Q-1,

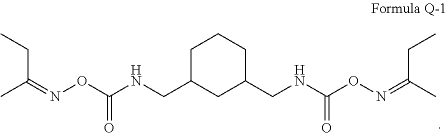 [SMILES: CC/C(C)=N\OC(=O)NCC1CCCC(CNC(=O)O/N=C(/C)CC)C1]

15. The blocked isocyanate compound according to claim 14,
wherein a mass ratio of a cis form to a trans form is cis form/trans form=10/90 to 90/10.

16. The transfer film according to claim 2,
wherein the blocked isocyanate compound has a ring structure.

17. The transfer film according to claim 2,
wherein the blocked isocyanate compound is a blocked isocyanate compound represented by Formula Q,
B¹-A¹-L¹-A²-B² Formula Q
in Formula Q, B¹ and B² each independently represent a blocked isocyanate group, A¹ and A² each independently represent a single bond or an alkylene group having 1 to 10 carbon atoms, and L¹ represents a divalent linking group.

18. The transfer film according to claim 3,
wherein the blocked isocyanate compound is a blocked isocyanate compound represented by Formula Q,
B¹-A¹-L¹-A²-B² Formula Q
in Formula Q, B¹ and B² each independently represent a blocked isocyanate group, A¹ and A² each independently represent a single bond or an alkylene group having 1 to 10 carbon atoms, and L¹ represents a divalent linking group.

19. The transfer film according to claim 2,
wherein the blocked isocyanate compound is a blocked isocyanate compound represented by Formula QA,
B^{1a}-A^{1a}-L^{1a}-A^{2a}-B^{2a} Formula QA
in Formula QA, B^{1a} and B^{2a} each independently represent a blocked isocyanate group, A^{1a} and A^{2a} each independently represent a divalent linking group, and L^{1a} represents a cyclic divalent saturated hydrocarbon group or a divalent aromatic hydrocarbon group.

20. The transfer film according to claim 3,
wherein the blocked isocyanate compound is a blocked isocyanate compound represented by Formula QA,
B^{1a}-A^{1a}-L^{1a}-A^{2a}-B^{2a} Formula QA
in Formula QA, B^{1a} and B^{2a} each independently represent a blocked isocyanate group, A^{1a} and A^{2a} each independently represent a divalent linking group, and L^{1a} represents a cyclic divalent saturated hydrocarbon group or a divalent aromatic hydrocarbon group.
